SEQUENCE LISTING (213 sequences)

SEQ ID NO: 1 - protein, 15 aa, Artificial Sequence
  Description of Artificial Sequence Synthetic peptide
NTRTSINIGRGQVFY

SEQ ID NO: 2 - DNA, 369 bp, Artificial Sequence
  Description of Artificial Sequence Synthetic polynucleotide
gaggtgcagctggtggagtctgggggaggcttggtcaagcctggagggtccctgagactctcctgtgcagcctctggattcacattcagtgactactacatgagctggatccgccaggctccagggaaggggctggagtgggtttcatacattagtagtagtgggaataccatatactacgcagactctgtgaagggccgattcaccatctccagggacaacgccaagaactctctgtatctgcaaatgaacagcctgagagccgaggacacggccgtgtattactgtgcgagagaggcgcggtggtggacgaaagggaacaactggttcgacccctggggccagggaaccctggtcaccgtctcctca

SEQ ID NO: 3 - DNA, 324 bp, Artificial Sequence
  Description of Artificial Sequence Synthetic polynucleotide
tcttatgagctgactcagccaccctcggtgtcagtggccccaggacagacggccagaattgcctgtgggggaaacaacattggaagtaaaagtgtacactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattgggtgttcggcggagggaccaagctgaccgtccta

SEQ ID NO: 4 - DNA, 354 bp, Artificial Sequence
  Description of Artificial Sequence Synthetic polynucleotide
caggtgcagctggtgcagtctggggctgaggtgaagaagcctggggctacagtgaaaatctcctgcaaggtttctggatacaccttcagcgacttgtacatgcactgggtgcaacaggcccctggaaatgggcttgagtggatgggatttgttgatcctgaagatggtgaaacaatatacgcagagaagttccagggcagactcaccataaccgcggacacgtctacagacacagcctacatggaactcagcagcctgagatctgaggacacggccgtgtattactgtgcaactggattactgggggaggctttcgatatctggggccaagggacaatggtcaccgtctcctca

SEQ ID NO: 5 - DNA, 333 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcactaatcggccctcagggctttctaatcgcttctctggctccaagtctggcaacacggcctccctgaccatctctgggctccaggctgaggacgaggctgattattactgcagctcatatacaagcaccagcactctttgggtgttcggcggagggaccaagctgaccgtccta

SEQ ID NO: 6 - DNA, 351 bp, Artificial Sequence
  Description of Artificial Sequence Synthetic polynucleotide
gaggttcagctggtggagtctgggggaggcttggtccagcctggggggtccctgagactctcctgtgcagcctctggaatcaccgtcagtaccacctacatgagctgggtccgccaggctccagggaagggcctggattgggtctcagttatttatagcgatggtagcacacactacgcagactccgtgaagggcagattcaccatctccagagacaactccaagaacacactgtttcttcaaatgaacagcctgagagccgaggacacggctgtgtattactgcgcgagccagtggactgcgtattgcaacttccgctggggccagggaaccctggtcaccgtctcctca

SEQ ID NO: 7 - DNA, 333 bp, Artificial Sequence
  Description of Artificial Sequence Synthetic polynucleotide
aattttatgctgactcagccccactctgtgtcggagtctccggggaagacggtagccatctcctgcacccgcagcagtggcagcattgccagcagctatgtgcagtggtaccagcagcgcccgggcagtgcccccaccactgtgatctatgaaaataaccaaagaccctctggggtccctgatcggttctctggctccatcgacagctcctccaactctgcctccctcaccatctctggactgaagactgaggacgaggctgactactactgtcagtcttatgatagcatcaatccttgggtgttcggcggagggaccaagctgaccgtccta

SEQ ID NO: 8 - DNA, 357 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactatgcacagatgtttcagggcagggtcaccatgaccagggacacgtccatcagcacagcctacatggagctgagcaggctgagatctgacgacacggccgtgttttactgtgcgacaggtggtagctggcttgggggtgttgactactggggccagggaaccctggtcaccgtctcctca

SEQ ID NO: 9 - DNA, 393 bp, Artificial Sequence
  Description of Artificial Sequence Synthetic polynucleotide
gctagcaccatggagacagacacactcctgctatgggtactgctgctctgggttccaggttccactggtgacgacattgtgctgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgacgattttgcaacttactactgtcaacggagttacagtacccctctgacgttcggccaagggaccaaggtggaaatcaaa

SEQ ID NO: 10 - DNA, 357 bp, Artificial Sequence; identical to SEQ ID NO: 8
  Description of Artificial Sequence Synthetic polynucleotide

SEQ ID NO: 11 - DNA, 330 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgacgttgggacttataactctgtctcctggtaccaacaacacccaggcaaagcccccaacctcataatttatgatgtcactaatcggccctcaggggtttctaatcgcttctctggctccaagtctggcaacacggcctccctgaccatctctgggctccagactgaggacgaggctgattattactgcagctcatatagaagaaccaacactctcgggttcggcggagggaccaagctgaccgtccta

SEQ ID NO: 12 - DNA, 438 bp, Artificial Sequence
  Description of Artificial Sequence Synthetic polynucleotide
gctagcaccatggagacagacacactcctgctatgggtactgctgctctgggttccaggttccactggtgaccaggtgcagctggtgcagtctggggctgaggtgaagaagcctggggcctcagtgaaactttcctgcaaggcatctggatacaccttcaacagctactatataaactggctgcgacaggcccctggacaagggcttgagtggatgggaataatcaaccctagtagtagtagcacaaactacgcacagaatttccagggcagagtcaccatgaccagggacacgtccacgagcacagtctacatggagctgagtagtctgagatctgaggacacggccgtctattactgtgcgagaaattatgctgggatagaagctcgaggttggctcgacccctggggccagggaaccctggtcaccgtctcctca

SEQ ID NO: 13 - DNA, 382 bp, Artificial Sequence
  Description of Artificial Sequence Synthetic polynucleotide
aagcttaccatggagacagacacactcctgctatgggtactgctgctctgggttccaggttccactggtgacgaaattgtgttgacgcagtctccaggcaccctgtctttgtctccaggggaaagagccaccctctcctgcagggccagtcagagtgttagcagcaggtccttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtatattactgtcagcagtcgaccactttcggcggagggaccaaggtggagatca

SEQ ID NO: 14 - DNA, 360 bp, Artificial Sequence
  Description of Artificial Sequence Synthetic polynucleotide
gaggtgcagctggtggagtctgggggaggcttggtccagcctggggggtccctgagactctcctgtgcagcctctggattcacatttagtgactattggatgagctgggtccgccaggctccagggaaggggctggagtgggtggccaacataaagtatgatggaagtgagaaatactatgtggactctgtgaagggccgattcaccacctccagagacaacgccaagaactcactgtatctgcaaatgaacagcctgagagccgaggacacggctgtgtattactgtgcaggattattatggttcggggagaaggcttttgatatctggggccaagggacaatggtcaccgtctcttca

SEQ ID NO: 15 - DNA, 321 bp, Artificial Sequence
  Description of Artificial Sequence Synthetic polynucleotide
gaaattgtgttgacacagtctccagccaccctgtctttgtctccaggggaaagagccaccctctcctgcagggccagtcagaatgttagcagatacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgctgtttattactgtcagcagcgtaggagctggcctcccactttcggcggagggaccaaggtggagatcaaa

SEQ ID NO: 16 - DNA, 381 bp, Artificial Sequence
  Description of Artificial Sequence Synthetic polynucleotide
gaggttcagctggtggagtctgggggaggcgtggtccagcctgggaggtccctgagactctcctgtgcagcctctggattcaccttcactagctatggcatgcactgggtccgccaggctccaggcaaggggctggagtgggtggcagttatatcaaatgatggaagtaatatatactatgcagactccgtggagggccgattcaccatctccagagacaatttcaagaacacggtgtatctgcaaatgaacagcctgggggctgaggacacggctgtgtactattgtgcgaaggctggcaattactatgatggtagtggttactactctcagtactactttgacaactggggccggggaaccctggtcaccgtctcctca

SEQ ID NO: 17 - DNA, 336 bp, Artificial Sequence
  Description of Artificial Sequence Synthetic polynucleotide
gacatcgtgatgacccagtctccagactccctggctgtgtctctgggcgagagggccaccgtcaactgcaagtccagccacagtgttttatacgactccaacagtaagaactacttagcttggtaccagcagaaaccaggacagcctcctaagctgctcatttactgggcatctacccgggattccggggtccctgaccgcttcagtggcagcgggtctgggacagagttcactctcaccatcagcagcctgcaggctgaagatgtggcactttattactgtcagcaatattacagtactcccactttcggcggagggaccaaggtggagatcaaa

SEQ ID NO: 18 - DNA, 361 bp, Artificial Sequence
  Description of Artificial Sequence Synthetic polynucleotide
caggtgcagctgcaggagtcgggcgcaggactgttgaagccttcggagaccctgtccctcacctgcgctgtctatggtgggtccttcagtggttactactggagctggatccgccagcccccagggaaggggctggagtggattggggaaatcattcatagtggaagcaccaactacaacccgtccctcaagagtcgagtcaccatatcagtagacacgtccaataaccagttctccttgaagctgagctctgtgaccgccgcggacacggctgtgtattactgtgcgagaggccgacgactactatggttcggggactttgactactggggccagggaaccctggtcaccgtctcctcag

SEQ ID NO: 19 - DNA, 322 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcggcagctacttagcctggtaccagcagaaacctggccaggctcccaggctcctcatctatggtgcatccagcagggccactggcatcccagacaggttcaatggcagtgggtctgggacagacttcactctcaccatcagcagactggagcctgaagattttgcagtgtattactgtcagcagtatggtagctcacctgcgttcggccaagggaccaaggtggaaatcaaac

SEQ ID NO: 20 - DNA, 360 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggccccaggacaagggcttgagtggatgggagggatcaactctaacagtggtggcacaaactttgcacagaagtttcagggcagggtcaccatgaccagggacacgtccatcagcacagcctacatggagctgagcaggctgagatctgacgacacggccgtgtattactgtgcgagcacatatagcagcacctggttccgctttgactactggggccagggaaccctggtcaccgtctcctca

SEQ ID NO: 21 - DNA, 327 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtattagcatcaactacttagcctggtaccagcagagacctggccaggctcccaggctcctcatctctggtgcatccagcagggccactggcatcccagacagattcagtggcagtgggtctgggacagacttcactctcaccatcagcagactggagcctgaagattttgcagtgtattactgtcagcagtatggtagctcacctccgtacacttttggccaggggaccaagctggagatcaaa

SEQ ID NO: 22 - DNA, 369 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcatcggctactatatgcactgggtgcgacaggcccctggacaagggcttgagtggatgggatggatcaaccctaacagcggtggcacaaactatgcacagaagtttcagggcagggtcaccatgaccagggacacgtccatcagaacagtctacatggagctgagcaggctgagatttgacgacacggccatgtattactgtgcgagagcccccagtctagtagtaggtgggggacggttggttgactactggggccagggatcgcaggtcaccgtctcctca

SEQ ID NO: 23 - DNA, 330 bp, Artificial Sequence
  Description of Artificial Sequence Synthetic polynucleotide
cagtctgccctgactcagcctcgctcagtgtccgggtctcctggacagtcagtcaccatctcctgcactggaaccagcagtgatgttggtggttataactatgtctcctggtgccaacagcacccaggcaaagccccccaactcatgatttatgatgtcagtaagcggccctcaggggtccctgatcgcttctctggctccaagtctggcaacatggcctccctgaccatctctgggctccaggctgaggatgagggtgattattattgctgctcatatgcaggcaattacactttggtattcggcggagggaccaggctgaccgtccta

SEQ ID NO: 24 - DNA, 315 bp, Artificial Sequence
  Description of Artificial Sequence Synthetic polynucleotide
caggtgcagctggtgcagtctgggcctgaggtgaagaagcctgggacctcaatgaagatctcctgcaaggcttctggattcacctttactaggtctactatgcagtgggtgcgacaggctcgtggacaacgccttgagtggataggatggatcgtcgttggcagtggtaacacaaactacgcacagaagttccaggaaagagtcaccattaccagggacatgtccacaagtacagcctacatggagctgagcagcctgagatccgaggacacggccgtgtactactgtgcggcagccccagtgggacctacctcg

SEQ ID NO: 25 - DNA, 322 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattatcaactatttaaattggtatcaacagaaaccagggagagcccctaagctcctgatctatgctgcatccagtttgctaagtggggtcccatcaaggttcagtggcagtggatctgggacagatttcactctcaccatcagcagtctgcaacctgaagattttgcaacttactactgtcaacagagttacagtaccccttacacttttggccaggggaccaagctggagatcaaac

SEQ ID NO: 26 - DNA, 367 bp, Artificial Sequence
  Description of Artificial Sequence Synthetic polynucleotide
caggtgcagctggtgcagtctggggctgaggtgaagaagcctggggcctcagtgaaagtttcctgcaaggcatctggatacatcttcatcagctactttatgcactgggtgcgacaggcccctggacaagggcttgagtggatgggaataatcaaccctagtagtggagacacaaggtatgcacagaagtttcagggcagagtcaccatgaccagggacacgtccacgaacacagtctacatggagctgagtagcctgagatctgacgacacggccgtgtattactgtgcgagaaggcccgggggactggaacgacacaattggttggacccctggggccagggaaccctggtcaccgtctcctcag

SEQ ID NO: 27 - DNA, 325 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtattagcagcaacttagcctggtaccagcagaaacctggccaggctcccaggctcctcatctatggtgcatccaccagggccactggtactccagccaggttcagtggcagtgggtctgggacagagttcactctcaccatcagcagcctgcagtctgaagattttgcatcttattactgtcagcagtataataactggcctgcgatcaccttcggccaagggacacgactggagattaaac

SEQ ID NO: 28 - DNA, 357 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctgggcaagggcttgagtggatgggacggatcaaccctaacactggtggcaccaactatgcacagaagtttcagggcagggtcatcatgaccagggacacgtcaatcaaaacaacctacatggagctaagcagcctgagatctgacgacatggccgtgtattactgtgcgaggtcggcaactggctactacggtatggacgcctggggccaagggaccacggtcaccgtctcctca

SEQ ID NO: 29 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattattaagtatttgaattggtatcaacaaagaccagggaaagcccctaagctcctgatctacgctacatccactttgcaaagtggggtcccagcaaggttcagtggcagtggatctgggacagatttcactctcaccatcagcagtctgcaacctgaagattttgcaacttactactgtcaacagagttacagtaccctctggacgttcggccaagggaccaaggtggagatcgaa

SEQ ID NO: 30 - DNA, 450 bp, Artificial Sequence
  Description of Artificial Sequence Synthetic polynucleotide
gctagcaccatggagacagacacactcctgctatgggtactgctgctctgggttccaggttccactggtgacgaggtgcagctggtgcagtctggagcagaggtgaaaaagcccggggagtctctgaagatctcctgtaagggttctggatacaggtttaccagttactggatcgtctgggtgcgccagatgcccgggaaaggcctggagtggatggggatcatctatcctggtgactttgataccaaatacagcccgtccttccaaggccaggtcaccatctcagccgacaagtccatcagcaccgcctacctacagtggagcagcctgaaggcctcggacaccgccatgtattactgtgcgagacttggcggcagatattaccatgatagtagtggttattactaccttgactactggggccagggaaccctggtcaccgtctcctca

SEQ ID NO: 31 - DNA, 330 bp, Artificial Sequence
  Description of Artificial Sequence Synthetic polynucleotide
aattttatgctgactcagccccactctgtgtcggagtctccggggaagacggtaaccatctcctgcacccgcagcagtggcagcgttgccagcgactatgtgcagtggtaccagcagcgcccgggcagtgcccccaccactgtggtctatgaggataaccaaagaccctctggggtccctgatcgattctctggctccatcgacagctcctccaactctgcctccctcaccatctctggactgaagactgaggacgaggctgactactactgtcagtcttatgataacagctcttgggtgttcggcggagggaccaagctgaccgtccta

SEQ ID NO: 32 - DNA, 432 bp, Artificial Sequence
  Description of Artificial Sequence Synthetic polynucleotide
gctagcaccatggagacagacacactcctgctatgggtactgctgctctgggttccaggttccactggtgacgaagtgcagctggtggagtctgggggaggcttggtacagcctggcaggtccctgagactctcctgtgcagcctctggattcacctttgatgatggtgccatgcactgggtccggcaagctccagggaagggcctggagtgggtctcaggtattagttggaatagtaatatcatagcctatgcggactctgtgaagggccgattcaccatctccagagacaacgccaagaactccctgtatttagaaatgaacagtctgagagttgaggacacggccttgtattactgtgcaaaagattctccgcggggggagctacccttgaactactggggccagggaaccctggtcaccgtctcctca

SEQ ID NO: 33 - DNA, 324 bp, Artificial Sequence
  Description of Artificial Sequence Synthetic polynucleotide
tcctatgagctgacacagccaccctcggtgtcagtgtccccaggacaaacggccaggatcacctgctctggagatgcattgccaaaaaactatgcttattggtaccagcagaagtcaggccaggcccctgtgttggtcatctatgaggacagcaaacgaccctccgggatccctgagagattctctggctccagctcagggacaatggccaccttgactatcagtggggcccaggttgaggatgaagctgactactactgttactcaacagacagcagtggtaatcatagggtgttcggcggagggaccaagctgaccgtccta

SEQ ID NO: 34 - DNA, 360 bp, Artificial Sequence
  Description of Artificial Sequence Synthetic polynucleotide
cagatcaccttgaaggagtctggtcctacgctggtgaaacccacacagaccctcacgctgacctgcaccttctctgggttctcactcagcattggtggagtgggtgtgggctggatccgtcagcccccaggaaaggccctggagtggcttgcactcatttattgggatgatgataagcgctacagcccatctctgaagagcagactcaccatcaccaaggacacctccaaaaaccaggtggtccttacaatgaccaacatgggccctgtggacacagccacatattactgcgccagattggttcggggaggtatatcctttgactactggggccagggaaccctggtcaccgtctcctca

SEQ ID NO: 35 - DNA, 309 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctgggtggcctggtatcagcagaaaccagggaaagcccctaagctcctgatctataagacatctagtttagaaagtggggtcccatcaaggttcagcggcagtggatctgggacagaattcactctcaccatcagcagcctgcagcctgatgattttgcaacttattactgccaagaaaggtggacgttcggccaagggaccaaggtggaaatcaaa

SEQ ID NO: 36 - DNA, 372 bp, Artificial Sequence
  Description of Artificial Sequence Synthetic polynucleotide
caggtgcagctggtgcagtctggggctgaggtgaagaagcctggggcctcagtgaaggtttcctgcaaggcttctggatacaatttcagtacctatgctatgcattgggtgcgccaggcccccggacaaaggcttgagtggatgggatggatcaacggtggcaatggtaagacaaaatattcacagaagttccagggcagagtcaccattaccagggacacttccgcgagcacagtctacatggacctgagcagcctgagatctgaagacacggctgtgtattactgtgcgagagcattttattactatgatagtcgtggttatttttccaatgacttctggggccagggaaccctggtcaccgtctcctct

SEQ ID NO: 37 - DNA, 324 bp, Artificial Sequence
  Description of Artificial Sequence Synthetic polynucleotide
tcctatgagctgacacagccaccctcggtgtcagtgtccccaggacaaacggccaggatcacctgctctggagatgcattgccaaaagaatatgcttattggtaccagcagaagtcaggccaggcccctgtgctggtcatctatgaggacagtgaacgaccctccgggatccctgagagattctctggctccagctcagggacaatggccaccttgactatcagtggggcccaggtggaggatgaagctgactactactgttactcaacagacagcagtggtgatctttgggtgttcggcggagggaccaagctgaccgtccta

SEQ ID NO: 38 - DNA, 357 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcatcggctactatatacactgggtgcgacaggcccctggacaagggcttgagtggatgggatggatcaaccctaacagtggtggcacaaactatgcacagaagtttcagggcagggtcaccatgaccagggacacgtccatcaccacagcctacatggagctgaccaggttgagatctgacgacacggccgtgtattattgtgcgagagggggtctcccaggaactggtacagcctactggggccagggaaccctggtcaccgtctcctca

SEQ ID NO: 39 - DNA, 324 bp, Artificial Sequence
  Description of Artificial Sequence Synthetic polynucleotide
gacatccagttgacccagtctccatcctccctgtctgcatctgtaggagacagagtcaccatcacttgccgggcgagtcagggcattagcaattatttagcctggtatcagcagaaaccagggaaaattcctaggctcctgatctatgctgcatccactttgcaatcaggggtcccatctcggttcagtggcagtggatctgggacagatttcactctcaccatcagcagcctgcagcctgaagatgttgcaacttattactgtcaaaagtttaacagtgtccctccgctcactttcggcggagggaccaaggtggagatcaaa

SEQ ID NO: 40 - protein, 123 aa, Artificial Sequence
  Description of Artificial Sequence Synthetic polypeptide
EVQLVESGGGLVKPGGSLRLSCAASGFTFSDYYMSWIRQAPGKGLEWVSYISSSGNTIYYADSVKGRFTISRDNAKNSLYLQMNSLRAEDTAVYYCAREARWWTKGNNWFDPWGQGTLVTVSS

SEQ ID NO: 41 - protein, 108 aa, Artificial Sequence
  Description of Artificial Sequence Synthetic polypeptide
SYELTQPPSVSVAPGQTARIACGGNNIGSKSVHWYQQKPGQAPVLVVYDDSDRPSGIPERFSGSNSGNTATLTISRVEAGDEADYYCQVWDSSSDHWVFGGGTKLTVL

SEQ ID NO: 42 - protein, 119 aa, Artificial Sequence
  Description of Artificial Sequence Synthetic polypeptide
QVQLVQSGAEVKKPGATVKISCKVSGYTFSDLYMHWVQQAPGNGLEWMGFVDPEDGETIYAEKFQGRLTITADTSTDTAYMELSSLRSEDTAVYYCATGLLGEAFDIWGQGTMVTVSSX

SEQ ID NO: 43 - protein, 111 aa, Artificial Sequence
  Description of Artificial Sequence Synthetic polypeptide
QSALTQPASVSGSPGQSITISCTGTSSDVGGYNYVSWYQQHPGKAPKLMIYDVTNRPSGLSNRFSGSKSGNTASLTISGLQAEDEADYYCSSYTSTSTLWVFGGGTKLTVL

SEQ ID NO: 44 - protein, 118 aa, Artificial Sequence
  Description of Artificial Sequence Synthetic polypeptide
EVQLVESGGGLVQPGGSLRLSCAASGITVSTTYMSWVRQAPGKGLDWVSVIYSDGSTHYADSVKGRFTISRDNSKNTLFLQMNSLRAEDTAVYYCASQWTAYCNFRWGQGTLVTVSSX

SEQ ID NO: 45 - protein, 111 aa, Artificial Sequence
  Description of Artificial Sequence Synthetic polypeptide
NFMLTQPHSVSESPGKTVAISCTRSSGSIASSYVQWYQQRPGSAPTTVIYENNQRPSGVPDRFSGSIDSSSNSASLTISGLKTEDEADYYCQSYDSINPWVFGGGTKLTVL

SEQ ID NO: 46 - protein, 119 aa, Artificial Sequence
  Description of Artificial Sequence Synthetic polypeptide
QVQLVQSGAEVKKPGASVKVSCKASGYTFTGYYIHWVRQAPGQGLEWMGWINPNSGGTNYAQMFQGRVTMTRDTSISTAYMELSRLRSDDTAVFYCATGGSWLGGVDYWGQGTLVTVSS

SEQ ID NO: 47 - protein, 131 aa, Artificial Sequence
  Description of Artificial Sequence Synthetic polypeptide
ASTMETDTLLLWVLLLWVPGSTGDDIVLTQSPSSLSASVGDRVTITCRASQSISSYLNWYQQKPGKAPKLLIYAASSLQSGVPSRFSGSGSGTDFTLTISSLQPDDFATYYCQRSYSTPLTFGQGTKVEIK

SEQ ID NO: 48 - protein, 119 aa, Artificial Sequence; identical to SEQ ID NO: 46
  Description of Artificial Sequence Synthetic polypeptide

SEQ ID NO: 49 - protein, 110 aa, Artificial Sequence
  Description of Artificial Sequence Synthetic polypeptide
QSALTQPASVSGSPGQSITISCTGTSSDVGTYNSVSWYQQHPGKAPNLIIYDVTNRPSGVSNRFSGSKSGNTASLTISGLQTEDEADYYCSSYRRTNTLGFGGGTKLTVL

SEQ ID NO: 50 - protein, 146 aa, Artificial Sequence
  Description of Artificial Sequence Synthetic polypeptide
ASTMETDTLLLWVLLLWVPGSTGDQVQLVQSGAEVKKPGASVKLSCKASGYTFNSYYINWLRQAPGQGLEWMGIINPSSSSTNYAQNFQGRVTMTRDTSTSTVYMELSSLRSEDTAVYYCARNYAGIEARGWLDPWGQGTLVTVSS

SEQ ID NO: 51 - protein, 128 aa, Artificial Sequence
  Description of Artificial Sequence Synthetic polypeptide
KLTMETDTLLLWVLLLWVPGSTGDEIVLTQSPGTLSLSPGERATLSCRASQSVSSRSLAWYQQKPGQAPRLLIYGASSRATGIPDRFSGSGSGTDFTLTISRLEPEDFAVYYCQQSTTFGGGTKVEIX

SEQ ID NO: 52 - protein, 120 aa, Artificial Sequence
  Description of Artificial Sequence Synthetic polypeptide
EVQLVESGGGLVQPGGSLRLSCAASGFTFSDYWMSWVRQAPGKGLEWVANIKYDGSEKYYVDSVKGRFTTSRDNAKNSLYLQMNSLRAEDTAVYYCAGLLWFGEKAFDIWGQGTMVTVSS

SEQ ID NO: 53 - protein, 107 aa, Artificial Sequence
  Description of Artificial Sequence Synthetic polypeptide
EIVLTQSPATLSLSPGERATLSCRASQNVSRYLAWYQQKPGQAPRLLIYDASNRATGIPARFSGSGSGTDFTLTISSLEPEDFAVYYCQQRRSWPPTFGGGTKVEIK

SEQ ID NO: 54 - protein, 127 aa, Artificial Sequence
  Description of Artificial Sequence Synthetic polypeptide
EVQLVESGGGVVQPGRSLRLSCAASGFTFTSYGMHWVRQAPGKGLEWVAVISNDGSNIYYADSVEGRFTISRDNFKNTVYLQMNSLGAEDTAVYYCAKAGNYYDGSGYYSQYYFDNWGRGTLVTVSS

SEQ ID NO: 55 - protein, 112 aa, Artificial Sequence
  Description of Artificial Sequence Synthetic polypeptide
DIVMTQSPDSLAVSLGERATVNCKSSHSVLYDSNSKNYLAWYQQKPGQPPKLLIYWASTRDSGVPDRFSGSGSGTEFTLTISSLQAEDVALYYCQQYYSTPTFGGGTKVEIK

SEQ ID NO: 56 - protein, 121 aa, Artificial Sequence
  Description of Artificial Sequence Synthetic polypeptide
QVQLQESGAGLLKPSETLSLTCAVYGGSFSGYYWSWIRQPPGKGLEWIGEIIHSGSTNYNPSLKSRVTISVDTSNNQFSLKLSSVTAADTAVYYCARGRRLLWFGDFDYWGQGTLVTVSSX

SEQ ID NO: 57 - protein, 108 aa, Artificial Sequence
  Description of Artificial Sequence Synthetic polypeptide
EIVLTQSPGTLSLSPGERATLSCRASQSVSGSYLAWYQQKPGQAPRLLIYGASSRATGIPDRFNGSGSGTDFTLTISRLEPEDFAVYYCQQYGSSPAFGQGTKVEIKX

SEQ ID NO: 58 - protein, 120 aa, Artificial Sequence
  Description of Artificial Sequence Synthetic polypeptide
QVQLVQSGAEVKKPGASVKVSCKASGYTFTGYYMHWVRQAPGQGLEWMGGINSNSGGTNFAQKFQGRVTMTRDTSISTAYMELSRLRSDDTAVYYCASTYSSTWFRFDYWGQGTLVTVSS

SEQ ID NO: 59 - protein, 109 aa, Artificial Sequence
  Description of Artificial Sequence Synthetic polypeptide
EIVLTQSPGTLSLSPGERATLSCRASQSISINYLAWYQQRPGQAPRLLISGASSRATGIPDRFSGSGSGTDFTLTISRLEPEDFAVYYCQQYGSSPPYTFGQGTKLEIK

SEQ ID NO: 60 - protein, 123 aa, Artificial Sequence
  Description of Artificial Sequence Synthetic polypeptide
QVQLVQSGAEVKKPGASVKVSCKASGYTFIGYYMHWVRQAPGQGLEWMGWINPNSGGTNYAQKFQGRVTMTRDTSIRTVYMELSRLRFDDTAMYYCARAPSLVVGGGRLVDYWGQGSQVTVSS

SEQ ID NO: 61 - protein, 110 aa, Artificial Sequence
  Description of Artificial Sequence Synthetic polypeptide
QSALTQPRSVSGSPGQSVTISCTGTSSDVGGYNYVSWCQQHPGKAPQLMIYDVSKRPSGVPDRFSGSKSGNMASLTISGLQAEDEGDYYCCSYAGNYTLVFGGGTRLTVL

SEQ ID NO: 62 - protein, 105 aa, Artificial Sequence
  Description of Artificial Sequence Synthetic polypeptide
QVQLVQSGPEVKKPGTSMKISCKASGFTFTRSTMQWVRQARGQRLEWIGWIVVGSGNTNYAQKFQERVTITRDMSTSTAYMELSSLRSEDTAVYYCAAAPVGPTS

SEQ ID NO: 63 - protein, 108 aa, Artificial Sequence
  Description of Artificial Sequence Synthetic polypeptide
DIQMTQSPSSLSASVGDRVTITCRASQSIINYLNWYQQKPGRAPKLLIYAASSLLSGVPSRFSGSGSGTDFTLTISSLQPEDFATYYCQQSYSTPYTFGQGTKLEIKX

SEQ ID NO: 64 - protein, 123 aa, Artificial Sequence
  Description of Artificial Sequence Synthetic polypeptide
QVQLVQSGAEVKKPGASVKVSCKASGYIFISYFMHWVRQAPGQGLEWMGIINPSSGDTRYAQKFQGRVTMTRDTSTNTVYMELSSLRSDDTAVYYCARRPGGLERHNWLDPWGQGTLVTVSSX

SEQ ID NO: 65 - protein, 109 aa, Artificial Sequence
  Description of Artificial Sequence Synthetic polypeptide
EIVMTQSPATLSVSPGERATLSCRASQSISSNLAWYQQKPGQAPRLLIYGASTRATGTPARFSGSGSGTEFTLTISSLQSEDFASYYCQQYNNWPAITFGQGTRLEIKX

SEQ ID NO: 66 - protein, 119 aa, Artificial Sequence
  Description of Artificial Sequence Synthetic polypeptide
QVQLVQSGAEVKKPGASVKVSCKASGYTFTGYYMHWVRQAPGQGLEWMGRINPNTGGTNYAQKFQGRVIMTRDTSIKTTYMELSSLRSDDMAVYYCARSATGYYGMDAWGQGTTVTVSS

SEQ ID NO: 67 - protein, 107 aa, Artificial Sequence
  Description of Artificial Sequence Synthetic polypeptide
DIQMTQSPSSLSASVGDRVTITCRASQSIIKYLNWYQQRPGKAPKLLIYATSTLQSGVPARFSGSGSGTDFTLTISSLQPEDFATYYCQQSYSTLWTFGQGTKVEIE

SEQ ID NO: 68 - protein, 150 aa, Artificial Sequence
  Description of Artificial Sequence Synthetic polypeptide
ASTMETDTLLLWVLLLWVPGSTGDEVQLVQSGAEVKKPGESLKISCKGSGYRFTSYWIVWVRQMPGKGLEWMGIIYPGDFDTKYSPSFQGQVTISADKSISTAYLQWSSLKASDTAMYYCARLGGRYYHDSSGYYYLDYWGQGTLVTVSS

SEQ ID NO: 69 - protein, 110 aa, Artificial Sequence
  Description of Artificial Sequence Synthetic polypeptide
NFMLTQPHSVSESPGKTVTISCTRSSGSVASDYVQWYQQRPGSAPTTVVYEDNQRPSGVPDRFSGSIDSSSNSASLTISGLKTEDEADYYCQSYDNSSWVFGGGTKLTVL

SEQ ID NO: 70 - protein, 144 aa, Artificial Sequence
  Description of Artificial Sequence Synthetic polypeptide
ASTMETDTLLLWVLLLWVPGSTGDEVQLVESGGGLVQPGRSLRLSCAASGFTFDDGAMHWVRQAPGKGLEWVSGISWNSNIIAYADSVKGRFTISRDNAKNSLYLEMNSLRVEDTALYYCAKDSPRGELPLNYWGQGTLVTVSS

SEQ ID NO: 71 - protein, 108 aa, Artificial Sequence
  Description of Artificial Sequence Synthetic polypeptide
SYELTQPPSVSVSPGQTARITCSGDALPKNYAYWYQQKSGQAPVLVIYEDSKRPSGIPERFSGSSSGTMATLTISGAQVEDEADYYCYSTDSSGNHRVFGGGTKLTVL

SEQ ID NO: 72 - protein, 120 aa, Artificial Sequence
  Description of Artificial Sequence Synthetic polypeptide
QITLKESGPTLVKPTQTLTLTCTFSGFSLSIGGVGVGWIRQPPGKALEWLALIYWDDDKRYSPSLKSRLTITKDTSKNQVVLTMTNMGPVDTATYYCARLVRGGISFDYWGQGTLVTVSS

SEQ ID NO: 73 - protein, 103 aa, Artificial Sequence
  Description of Artificial Sequence Synthetic polypeptide
DIQMTQSPSTLSASVGDRVTITCRASQSISSWVAWYQQKPGKAPKLLIYKTSSLESGVPSRFSGSGSGTEFTLTISSLQPDDFATYYCQERWTFGQGTKVEIK

SEQ ID NO: 74 - protein, 124 aa, Artificial Sequence
  Description of Artificial Sequence Synthetic polypeptide
QVQLVQSGAEVKKPGASVKVSCKASGYNFSTYAMHWVRQAPGQRLEWMGWINGGNGKTKYSQKFQGRVTITRDTSASTVYMDLSSLRSEDTAVYYCARAFYYYDSRGYFSNDFWGQGTLVTVSS

SEQ ID NO: 75 - protein, 108 aa, Artificial Sequence
  Description of Artificial Sequence Synthetic polypeptide
SYELTQPPSVSVSPGQTARITCSGDALPKEYAYWYQQKSGQAPVLVIYEDSERPSGIPERFSGSSSGTMATLTISGAQVEDEADYYCYSTDSSGDLWVFGGGTKLTVL

SEQ ID NO: 76 - protein, 119 aa, Artificial Sequence
  Description of Artificial Sequence Synthetic polypeptide
QVQLVQSGAEVKKPGASVKVSCKASGYTFIGYYIHWVRQAPGQGLEWMGWINPNSGGTNYAQKFQGRVTMTRDTSITTAYMELTRLRSDDTAVYYCARGGLPGTGTAYWGQGTLVTVSS

SEQ ID NO: 77 - protein, 108 aa, Artificial Sequence
  Description of Artificial Sequence Synthetic polypeptide
DIQLTQSPSSLSASVGDRVTITCRASQGISNYLAWYQQKPGKIPRLLIYAASTLQSGVPSRFSGSGSGTDFTLTISSLQPEDVATYYCQKFNSVPPLTFGGGTKVEIK

SEQ ID NO: 78 - DNA, 366 bp, Artificial Sequence
  Description of Artificial Sequence Synthetic polynucleotide
caggtgcagctggtgcagtctggggctgaggtgcagaagcctgggtcctcggtgaaggtctcctgcaaggcttctggaggcaccttcagcacctatagtatcagctgggtgcgacaggcccctggacaagggcttgagtggatgggaaggagcatccctatccttggtatagcaaattacgcacagaagttccagggcagagtcacgtttaccgcggacaaatccacgaccacagcctacatggagctgagcagcctgagatctgaggacacggccgtgtattactgtgcgagaggggtaggtcagcagctcgtccagtactactttgactactggggccagggaaccctggtcaccgtctcctca

SEQ ID NO: 79 - DNA, 330 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgaggtcagtaagcggccctcaggggtttctaatcgcttctctggctccaagtctggcaacacggcctccctgacaatctctgggctcctggctgaggacgaggctgattattactgctgctcatatgcaggtagtagtatttcagtgttcggcggagggaccaagctgaccgtccta

SEQ ID NO: 80 - DNA, 369 bp, Artificial Sequence
  Description of Artificial Sequence Synthetic polynucleotide
caggtgcagctgtgcggagtcgggccaggactggtgaagccttcacagaccttgtccctcagttgcactgtctctggtggctccagcagtagtggtgctcactactggagttggatccgccagtacccagggaagggcctggagtggattggttacatccattacagtgggaacacttactacaacccgtccctcaagagtcgaattaccatatcacaacacacgtctgagaaccagttctccctgaagctcaactctgtgactgttgcagacacggccgtctattactgtgcgagagggacccgtctccggacactacggaatgcttttgatatttggggccaggggacaagggtcaccgtctcttca

SEQ ID NO: 81 - DNA, 330 bp, Artificial Sequence
  Description of Artificial Sequence Synthetic polynucleotide
cagtctgccctgactcagcctccctccgcgtccgggtctcctggacagtcagtcaccatctcctgcactggaaccagcagtgacgttggtggttataactatgtttcctggtaccaacaccacccaggcaaagcccccaaactcataatttctgaggtcaataaccggccctcaggggtccctgatcgtttctctggctccaagtctggcaacacggcctccctgaccgtctctgggctccaggctgaggatgaggctgaatattactgcagctcatacacagacatccacaatttcgtcttcggcggagggaccaagctgaccgtccta

SEQ ID NO: 82 - protein, 122 aa, Artificial Sequence
  Description of Artificial Sequence Synthetic polypeptide
QVQLVQSGAEVQKPGSSVKVSCKASGGTFSTYSISWVRQAPGQGLEWMGRSIPILGIANYAQKFQGRVTFTADKSTTTAYMELSSLRSEDTAVYYCARGVGQQLVQYYFDYWGQGTLVTVSS

SEQ ID NO: 83 - protein, 110 aa, Artificial Sequence
  Description of Artificial Sequence Synthetic polypeptide
QSALTQPASVSGSPGQSITISCTGTSSDVGSYNLVSWYQQHPGKAPKLMIYEVSKRPSGVSNRFSGSKSGNTASLTISGLLAEDEADYYCCSYAGSSISVFGGGTKLTVL

SEQ ID NO: 84 - protein, 123 aa, Artificial Sequence
  Description of Artificial Sequence Synthetic polypeptide
QVQLCGVGPGLVKPSQTLSLSCTVSGGSSSSGAHYWSWIRQYPGKGLEWIGYIHYSGNTYYNPSLKSRITISQHTSENQFSLKLNSVTVADTAVYYCARGTRLRTLRNAFDIWGQGTRVTVSS

SEQ ID NO: 85 - protein, 110 aa, Artificial Sequence
  Description of Artificial Sequence Synthetic polypeptide
QSALTQPPSASGSPGQSVTISCTGTSSDVGGYNYVSWYQHHPGKAPKLIISEVNNRPSGVPDRFSGSKSGNTASLTVSGLQAEDEAEYYCSSYTDIHNFVFGGGTKLTVL

SEQ ID NO: 86 - DNA, 378 bp, Artificial Sequence
  Description of Artificial Sequence Synthetic polynucleotide
caggtgcagctggtgcagtctgggggaggcgttttcaagcctggagggtccctgagactctcctgtgaagcctctggattcacatttactgaatattacatgacttgggtccgccaggctcctgggaaggggctggagtggcttgcgtatattagtaagaatggtgaatattcaaaatattcaccgtcctcaaacggccggttcaccatctccagagacaacgccaagaactcagtgtttctgcaattggacagactgagcgccgacgacacggccgtctattactgtgcgagagcggacggattaacatacttctctgaattactccaatacatttttgacctctggggccagggagcccgggtcaccgtctcctcg

SEQ ID NO: 87 - protein, 147 aa, Artificial Sequence
  Description of Artificial Sequence Synthetic polypeptide
METDTLLLWVLLLWVPGSTGDQVQLVQSGGGVFKPGGSLRLSCEASGFTFTEYYMTWVRQAPGKGLEWLAYISKNGEYSKYSPSSNGRFTISRDNAKNSVFLQLDRLSADDTAVYYCARADGLTYFSELLQYIFDLWGQGARVTVSS

SEQ ID NO: 88 - protein, 134 aa, Artificial Sequence
  Description of Artificial Sequence Synthetic polypeptide
METDTLLLWVLLLWVPGSTGDETTLTQSPDSLAVSPGERATIHCKSSQTLLYSSNNRHSIAWYQQRPGQPPKLLLYWASMRLSGVPDRFSGSGSGTDFTLTINNLQAEDVAIYYCHQYSSHPPTFGHGTRVELR

SEQ ID NO: 89 - DNA, 339 bp, Artificial Sequence
  Description of Artificial Sequence Synthetic polynucleotide
gaaacgacactcacgcagtctccagactccctggctgtgtctccgggcgagagggccaccatccactgcaagtccagccagactcttttgtacagctccaacaatagacactccattgcttggtaccaacagagaccaggacagcctcctaaattactcctttattgggcatctatgcggctttccggggtccctgaccgattcagtggcagcgggtctgggacagatttcactctcaccatcaacaacctgcaggctgaggatgtggccatctattactgtcaccaatattccagtcatcccccgacgttcggccacgggaccagggtggagctcaga

SEQ ID NO: 90 - DNA, 369 bp, Artificial Sequence
  Description of Artificial Sequence Synthetic polynucleotide
caggtgcagctggtgcagtctggggctgaggtgaagaagcctggggcctcagtgaaggtctcctgcaaggcctctgaatacagcttcaccggctactatttgcactgggtgcgccaggcccctggacaagggcttgagtggatgggatggatcaatcctaacaatggtgacacaagatctgcacagaggtttcagggcagggtcaccatgaccagggacacgtccatcagcacagcctacatggaagtgagcagcctgacatctgacgacgcggccatatattactgtgcgagagcccggtgggacctactaccgggagggcgctgctttgactactggggccagggaaccctggtcaccgtctcctca

SEQ ID NO: 91 - DNA, 330 bp, Artificial Sequence
  Description of Artificial Sequence Synthetic polynucleotide
cagtctgccctgactcagcctcgctcagtgtccgggtctcctgggcagtcagtcaccatctcctgcactggaaccagcagtgatgttggtagttatacttatgtctcctggtaccaacagcacccaggcaaagcccccaaactcattgtttctgatgtcagtgagcggccctcaggggtccctgatcgcttctctggctccaagtctggcaacacggcctccctgaccatctctgggctccaggctgaggatgaggctgattattattgctcctcatatgcaggcagctacactttcgtgttcggcggagggaccaaggtgaccgtccta

SEQ ID NO: 92 - DNA, 366 bp, Artificial Sequence
  Description of Artificial Sequence Synthetic polynucleotide
caggtgcagctggtacagtctggggctgaggtgaagaagcctggggcctcagtgaaggtctcctgcaaggcttctggttacaccttcatcgcctactacatgcactgggtgcgacaggcccctggacaagggcttgagtggatgggatggatcaaccctaacagtggaggcacaaactatgcacagaagtttcagggcagggtcaccatgaccagggacacgtccatcagcacagcctacatggagctgagcaggctgagatttgacgacacggccgtgtactattgtgggagatttagtggaaactactttttgtatcacggtatggacgtctggggccaagggaccacggtcaccgtctcctca

SEQ ID NO: 93 - DNA, 336 bp, Artificial Sequence
  Description of Artificial Sequence Synthetic polynucleotide
gacatcgtgatgacccagtctccagactccctggctctgtctctgggcgagagggccaccatcaactgcaagtccagccagagtgttttatacaactccaacaataagaactacttagcttggtaccagcagaaaccaggacagcctcctaagctactcatttactgggcatctacccgggaatccggggtccctgaccgattcagtggcagcgggtctgggacagatttcactctcaccatcagcagcctgcaggctgaagatgtggcagtttattactgtcagcaatattatagtaatctcactttcggcggagggaccaaggtggagatcaaa

SEQ ID NO: 94 - DNA, 378 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagtacagcctacatggagctgagcaggctgagatctgacgacacggccgtgtatttctgtgcgagagtttgggtttattactatgatagtagtggttattcctacccctttgactactggggccagggaaccctggtcaccgtctcctca

SEQ ID NO: 95 - DNA, 321 bp, Artificial Sequence
  Description of Artificial Sequence Synthetic polynucleotide
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaagagggccactggcatcccagccaggttcagtgtcagtgggtctgggacagacttcactctcaccatcagcagcctagagcctgaagattttgcagtttattactgtcagcagcgtagccactggacgtggacgttcggccaagggaccagggtggaaatcaaa

SEQ ID NO: 96 - DNA, 378 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtgcatgggatggatgaaccctaacagtggtaacacaggctatgcacagaagttccagggcagagtcaccatgaccaggaacacctccataagcacagcctacatggagctgagcagcctgagatctgacgacacggccgtgtattactgtgcgagaggcctccgaacgtattactatggttcggggtattatcgccccttggggtactggggccagggaaccctggtcaccgtctcctca

SEQ ID NO: 97 - DNA, 321 bp, Artificial Sequence
  Description of Artificial Sequence Synthetic polynucleotide
gacatccagatgacccagtctccatcctccctgtctgcatctgttggagacagggtcaccatcacttgccgggcaagtcagagcattagcaactatttaaattggtatcagcagaaaccacggaaagcccctaagctcctgatctatgctgcatccagtttgcaaagtggggtcccatcaaggttcagtggcagtggatctggggcagatttcactctcaccatcagcagtctgcaacctgaagattttgcaacttactactgtcaacagagttacagtaccctccaaacttttggccaggggaccaagctggagatcaaa

SEQ ID NO: 98 - DNA, 357 bp, Artificial Sequence
  Description of Artificial Sequence Synthetic polynucleotide
gaggttcagctggtggagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcaccgaagtttcagggcagggtcaccatgaccagggacacgtccatcagcacagcctacatggaattgaccgggctgagatctgacgacacggccgtctattactgtgcgaggagcattactttgatagtaaactttgcctactggggccagggaaccctggtcaccgtctcctca

SEQ ID NO: 99 - DNA, 321 bp, Artificial Sequence
  Description of Artificial Sequence Synthetic polynucleotide
gacattgtgctgacccagtctccatcctccctgtctgcatctgtaggagacagagtcaccatcacttgccggacaagtcagagcattagcaactatttaaattggtatcagcagaaaccagggaaagcccctaagctcctgatctatgctgcatccagtttgcaaagtggggtcccatcaaggttcagtggcagtggatctgggacagatttcactctcaccgtcagcagtctgcaacctgaagattttgcaacttactactgtcaacagacttacaatacccctgtcactttcggcggagggaccaaggtggagatcaaa

SEQ ID NO: 100 - DNA, 363 bp, Artificial Sequence
  Description of Artificial Sequence Synthetic polynucleotide
caggtgcagctggtgcagtctggggctgaggtgaagaagcctggggcctcagtgaaggtttcctgcaaggcatctggatacaccttcatcagttactatatgcactgggtgcgacaggcccctggacaagggcttgagtggatgggaataatcaactctagtggtggtagtacaaactacgcacagaagttccagggcagagtcaccatgaccagggacacgtccacgagcacagtctacatggagctgagcagcctgagatctgaggacacggccgtgtattactgtgcgagaggtggtattactttggttcggggagttatttactactggggccagggaaccctggtcaccgtctcctca

SEQ ID NO: 101 - DNA, 321 bp, Artificial Sequence
  Description of Artificial Sequence Synthetic polynucleotide
gaaattgtgttgacacagtctccatcctccctgtctgcatctgtaggagacagagtcaccatcacttgccgggcgagtcagggcattagcaattctttagcctggtatcagcagaaaccagggaaagcccctaagctcctgctctatgctgcatccagattggaaagtggggtcccatccaggttcagtggcagtggatctgggacggattacactctcaccatcagcagcctccagcctgaagattttgcaacttattactgtcaacagtattatagtaccctcccgacgttcggccaagggaccaaggtggaaatcaaa

SEQ ID NO: 102 - DNA, 369 bp, Artificial Sequence
  Description of Artificial Sequence Synthetic polynucleotide
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggaacatctattacagtgggagcacctactacaacccctccctcaagagtcgaattactatatcactagacacgtctaagaaccagttctccctgaagctgagctctgtgacggccgcggacacggccgtgtattactgtgcgaggacctctcggacaacggtcctaaggaatgcttttgatatctggggccacgggacaatggtcaccgtctcttca

SEQ ID NO: 103 - DNA, 333 bp, Artificial Sequence
  Description of Artificial Sequence Synthetic polynucleotide
cagtctgccctgactcagcctcgctcagtgtccgggtctcctggacagtcagtcaccatctcctgcactggaaccagcagtgatgttggtggttatgactatgtctcctggtaccagcagcacccaggcaaagcccccaaactcatgatttctgatgtcagtaagcggccctcaggggtccctgatcgcttctctggctccaagtctggcaacacggcctccctgaccatctctgggctccagcctgaggatgaggctgattattactgctgctcatatgcaggcacctacacttattgggtgttcggcggagggaccaagctgaccgtccta

SEQ ID NO: 104 - DNA, 378 bp, Artificial Sequence
  Description of Artificial Sequence Synthetic polynucleotide
caggtgcagctggtgcagtctggggctgaggtgaagaagcctggggcctcagtgaaggtctcctgcaaggcttctggatacaccttcaccggctactatatacactgggtgcgacaggcccctggacaagggcttgagtggatgggatggatcaaccctaacagtggtggcacaaactatgcacagaagtttcagggcagggtcaccatgaccagggacacgtccatcagcgcagcctacatggagctgagcagcctgctatttgacgacacggccgtgtattactgtgcgagagtgccccatattgtagtggtggtagctgctgccaatgcgaactttgactactggggccagggaaccctggtcaccgtcgcctca

SEQ ID NO: 105 - DNA, 321 bp, Artificial Sequence
  Description of Artificial Sequence Synthetic polynucleotide
gacatccagatgacccagtctccatcctctgtgtctgcatctgtaggagacagagtcaccatcacttgtcgggcgagtcagggtattagtacttggttagcctggtatcagcagaaaccagggaaagcccctaaggccctgatctatgctgcaaccagattgcaaagtggggtcccatcaaggttcagcggcagtggatctgggacagatttcactctcactatcagcagcctgcagcctgaagattttgcaatttactattgtcaacaggctaacagtttccccatcaccttcggccaagggacacgactggagattaaa

SEQ ID NO: 106 - DNA, 390 bp, Artificial Sequence
  Description of Artificial Sequence Synthetic polynucleotide
gaggttcagctggtggagtctggggctgaggtgaagaagcctggggcctcagtgcaggtttcctgcaaggcatctggatacatcttcaaaagctattatgtgcactgggtgcgacaggcccctggacaagggcttgagtggatgggaataatcaaccctagtggtggtaccgcaagttacgcacagaagttccagggcagagtcaccatgaccagggacacgtccacgagcacagtctacatggagctgagcagcctgagatctgaggacacggccgtgtattactgtgcgagagataacgggattgttgggtatagtggctcccgggggtattactactactacggtatggacgtctggggccaagggaccacggtcaccgtctcctca

SEQ ID NO: 107 - DNA, 321 bp, Artificial Sequence
  Description of Artificial Sequence Synthetic polynucleotide
gaaattgtgttgacacagtctccatcctccctgtctgcatctgttggagacagagtcaccttcacttgccgggcaagtcagagcatgagcagctgtttaaattggtatcagcaaaaacccgggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccttacacttttggccaggggaccaagctggagatcaac

SEQ ID NO: 108 - protein, 123 aa, Artificial Sequence
  Description of Artificial Sequence Synthetic polypeptide
QVQLVQSGAEVKKPGASVKVSCKASEYSFTGYYLHWVRQAPGQGLEWMGWINPNNGDTRSAQRFQGRVTMTRDTSISTAYMEVSSLTSDDAAIYYCARARWDLLPGGRCFDYWGQGTLVTVSS

SEQ ID NO: 109 - protein, 110 aa, Artificial Sequence
  Description of Artificial Sequence Synthetic polypeptide
QSALTQPRSVSGSPGQSVTISCTGTSSDVGSYTYVSWYQQHPGKAPKLIVSDVSERPSGVPDRFSGSKSGNTASLTISGLQAEDEADYYCSSYAGSYTFVFGGGTKVTVL

SEQ ID NO: 110 - protein, 122 aa, Artificial Sequence
  Description of Artificial Sequence Synthetic polypeptide
QVQLVQSGAEVKKPGASVKVSCKASGYTFIAYYMHWVRQAPGQGLEWMGWINPNSGGTNYAQKFQGRVTMTRDTSISTAYMELSRLRFDDTAVYYCGRFSGNYFLYHGMDVWGQGTTVTVSS

SEQ ID NO: 111 - protein, 112 aa, Artificial Sequence
  Description of Artificial Sequence Synthetic polypeptide
DIVMTQSPDSLALSLGERATINCKSSQSVLYNSNNKNYLAWYQQKPGQPPKLLIYWASTRESGVPDRFSGSGSGTDFTLTISSLQAEDVAVYYCQQYYSNLTFGGGTKVEIK

SEQ ID NO: 112 - protein, 126 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYFCARVWVYYYDSSGYSYPFDYWGQGTLVTVSS

SEQ ID NO: 113 - protein, 107 aa, Artificial Sequence
  Description of Artificial Sequence Synthetic polypeptide
EIVLTQSPATLSLSPGERATLSCRASQSVSSYLAWYQQKPGQAPRLLIYDASKRATGIPARFSVSGSGTDFTLTISSLEPEDFAVYYCQQRSHWTWTFGQGTRVEIK

SEQ ID NO: 114 - protein, 126 aa, Artificial Sequence
  Description of Artificial Sequence Synthetic polypeptide
QVQLVQSGAEVKKPGASVKVSCKASGYTFTSYDINWVRQATGQGLECMGWMNPNSGNTGYAQKFQGRVTMTRNTSISTAYMELSSLRSDDTAVYYCARGLRTYYYGSGYYRPLGYWGQGTLVTVSS

SEQ ID NO: 115 - protein, 107 aa, Artificial Sequence
  Description of Artificial Sequence Synthetic polypeptide
DIQMTQSPSSLSASVGDRVTITCRASQSISNYLNWYQQKPRKAPKLLIYAASSLQSGVPSRFSGSGSGADFTLTISSLQPEDFATYYCQQSYSTLQTFGQGTKLEIK

SEQ ID NO: 116 - protein, 119 aa, Artificial Sequence
  Description of Artificial Sequence Synthetic polypeptide
EVQLVESGAEVKKPGASVKVSCKASGYTFTGYYMHWVRQAPGQGLEWMGWINPNSGGTNYAPKFQGRVTMTRDTSISTAYMELTGLRSDDTAVYYCARSITLIVNFAYWGQGTLVTVSS

SEQ ID NO: 117 - protein, 107 aa, Artificial Sequence
  Description of Artificial Sequence Synthetic polypeptide
DIVLTQSPSSLSASVGDRVTITCRTSQSISNYLNWYQQKPGKAPKLLIYAASSLQSGVPSRFSGSGSGTDFTLTVSSLQPEDFATYYCQQTYNTPVTFGGGTKVEIK

SEQ ID NO: 118 - protein, 121 aa, Artificial Sequence
  Description of Artificial Sequence Synthetic polypeptide
QVQLVQSGAEVKKPGASVKVSCKASGYTFISYYMHWVRQAPGQGLEWMGIINSSGGSTNYAQKFQGRVTMTRDTSTSTVYMELSSLRSEDTAVYYCARGGITLVRGVIYYWGQGTLVTVSS

SEQ ID NO: 119 - protein, 107 aa, Artificial Sequence
  Description of Artificial Sequence Synthetic polypeptide
EIVLTQSPSSLSASVGDRVTITCRASQGISNSLAWYQQKPGKAPKLLLYAASRLESGVPSRFSGSGSGTDYTLTISSLQPEDFATYYCQQYYSTLPTFGQGTKVEIK

SEQ ID NO: 120 - protein, 123 aa, Artificial Sequence
  Description of Artificial Sequence Synthetic polypeptide
QVQLQESGPGLVKPSQTLSLTCTVSGGSISSGGYYWSWIRQHPGKGLEWIGNIYYSGSTYYNPSLKSRITISLDTSKNQFSLKLSSVTAADTAVYYCARTSRTTVLRNAFDIWGHGTMVTVSS

SEQ ID NO: 121 - protein, 111 aa, Artificial Sequence
  Description of Artificial Sequence Synthetic polypeptide
QSALTQPRSVSGSPGQSVTISCTGTSSDVGGYDYVSWYQQHPGKAPKLMISDVSKRPSGVPDRFSGSKSGNTASLTISGLQPEDEADYYCCSYAGTYTYWVFGGGTKLTVL

SEQ ID NO: 122 - protein, 126 aa, Artificial Sequence
  Description of Artificial Sequence Synthetic polypeptide
QVQLVQSGAEVKKPGASVKVSCKASGYTFTGYYIHWVRQAPGQGLEWMGWINPNSGGTNYAQKFQGRVTMTRDTSISAAYMELSSLLFDDTAVYYCARVPHIVVVVAAANANFDYWGQGTLVTVAS

SEQ ID NO: 123 - protein, 107 aa, Artificial Sequence
  Description of Artificial Sequence Synthetic polypeptide
DIQMTQSPSSVSASVGDRVTITCRASQGISTWLAWYQQKPGKAPKALIYAATRLQSGVPSRFSGSGSGTDFTLTISSLQPEDFAIYYCQQANSFPITFGQGTRLEIK

SEQ ID NO: 124 - protein, 130 aa, Artificial Sequence
  Description of Artificial Sequence Synthetic polypeptide
EVQLVESGAEVKKPGASVQVSCKASGYIFKSYYVHWVRQAPGQGLEWMGIINPSGGTASYAQKFQGRVTMTRDTSTSTVYMELSSLRSEDTAVYYCARDNGIVGYSGSRGYYYYYGMDVWGQGTTVTVSS

SEQ ID NO: 125 - protein, 107 aa, Artificial Sequence
  Description of Artificial Sequence Synthetic polypeptide
EIVLTQSPSSLSASVGDRVTFTCRASQSMSSCLNWYQQKPGKAPKLLIYAASSLQSGVPSRFSGSGSGTDFTLTISSLQPEDFATYYCQQSYSTPYTFGQGTKLEIN

SEQ ID NO: 126 - DNA, 369 bp, Artificial Sequence; identical to SEQ ID NO: 2
  Description of Artificial Sequence Synthetic polynucleotide

SEQ ID NO: 127 - DNA, 324 bp, Artificial Sequence; identical to SEQ ID NO: 3
  Description of Artificial Sequence Synthetic polynucleotide

SEQ ID NO: 128 - DNA, 354 bp, Artificial Sequence; identical to SEQ ID NO: 4
  Description of Artificial Sequence Synthetic polynucleotide

SEQ ID NO: 129 - DNA, 333 bp, Artificial Sequence; identical to SEQ ID NO: 5
  Description of Artificial Sequence Synthetic polynucleotide

SEQ ID NO: 130 - DNA, 351 bp, Artificial Sequence; identical to SEQ ID NO: 6
  Description of Artificial Sequence Synthetic polynucleotide

SEQ ID NO: 131 - DNA, 333 bp, Artificial Sequence; identical to SEQ ID NO: 7
  Description of Artificial Sequence Synthetic polynucleotide

SEQ ID NO: 132 - DNA, 357 bp, Artificial Sequence; identical to SEQ ID NO: 8
  Description of Artificial Sequence Synthetic polynucleotide

SEQ ID NO: 133 - DNA, 393 bp, Artificial Sequence; identical to SEQ ID NO: 9
  Description of Artificial Sequence Synthetic polynucleotide

SEQ ID NO: 134 - DNA, 358 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcaataatgccatgagttgggtccgccagactccagggaaggggctggagtgggtctcaagttttagtggtggacgtgataccacatactacgcagactccgtgaagggccggttcaccatctccagagacgattccaagaacacgctgtttctgcaaatgagcagcctgagagccgaggacacggccgtatattactgcgcgaaagatctgggtctgcttcggggaattgcaaactggggccagggaaccctggtcaccgtctcctcag

SEQ ID NO: 135 - DNA, 330 bp, Artificial Sequence; identical to SEQ ID NO: 11
  Description of Artificial Sequence Synthetic polynucleotide

SEQ ID NO: 136 - DNA, 438 bp, Artificial Sequence; identical to SEQ ID NO: 12
  Description of Artificial Sequence Synthetic polynucleotide

SEQ ID NO: 137 - DNA, 382 bp, Artificial Sequence; identical to SEQ ID NO: 13
  Description of Artificial Sequence Synthetic polynucleotide

SEQ ID NO: 138 - DNA, 360 bp, Artificial Sequence; identical to SEQ ID NO: 14
  Description of Artificial Sequence Synthetic polynucleotide

SEQ ID NO: 139 - DNA, 321 bp, Artificial Sequence; identical to SEQ ID NO: 15
  Description of Artificial Sequence Synthetic polynucleotide

SEQ ID NO: 140 - DNA, 381 bp, Artificial Sequence; identical to SEQ ID NO: 16
  Description of Artificial Sequence Synthetic polynucleotide

SEQ ID NO: 141 - DNA, 336 bp, Artificial Sequence; identical to SEQ ID NO: 17
  Description of Artificial Sequence Synthetic polynucleotide

SEQ ID NO: 142 - DNA, 361 bp, Artificial Sequence; identical to SEQ ID NO: 18
  Description of Artificial Sequence Synthetic polynucleotide

SEQ ID NO: 143 - DNA, 322 bp, Artificial Sequence; identical to SEQ ID NO: 19
  Description of Artificial Sequence Synthetic polynucleotide

SEQ ID NO: 144 - DNA, 360 bp, Artificial Sequence; identical to SEQ ID NO: 20
  Description of Artificial Sequence Synthetic polynucleotide

SEQ ID NO: 145 - DNA, 327 bp, Artificial Sequence; identical to SEQ ID NO: 21
  Description of Artificial Sequence Synthetic polynucleotide

SEQ ID NO: 146 - DNA, 369 bp, Artificial Sequence; identical to SEQ ID NO: 22
  Description of Artificial Sequence Synthetic polynucleotide

SEQ ID NO: 147 - DNA, 330 bp, Artificial Sequence; identical to SEQ ID NO: 23
  Description of Artificial Sequence Synthetic polynucleotide

SEQ ID NO: 148 - DNA, 364 bp, Artificial Sequence
  Description of Artificial Sequence Synthetic polynucleotide
caggtgcagctggtgcagtctgggcctgaggtgaagaagcctgggacctcaatgaagatctcctgcaaggcttctggattcacctttactaggtctactatgcagtgggtgcgacaggctcgtggacaacgccttgagtggataggatggatcgtcgttggcagtggtaacacaaactacgcacagaagttccaggaaagagtcaccattaccagggacatgtccacaagtacagcctacatggagctgagcagcctgagatccgaggacacggccgtgtactactgtgcggcagccccagtgggacctacctcgaactggtttgacccctggggccagggaaccctggtcaccgtctcctcag

SEQ ID NO: 149 - DNA, 322 bp, Artificial Sequence; identical to SEQ ID NO: 25
  Description of Artificial Sequence Synthetic polynucleotide

SEQ ID NO: 150 - DNA, 367 bp, Artificial Sequence; identical to SEQ ID NO: 26
  Description of Artificial Sequence Synthetic polynucleotide

SEQ ID NO: 151 - DNA, 325 bp, Artificial Sequence; identical to SEQ ID NO: 27
  Description of Artificial Sequence Synthetic polynucleotide

SEQ ID NO: 152 - DNA, 357 bp, Artificial Sequence; identical to SEQ ID NO: 28
  Description of Artificial Sequence Synthetic polynucleotide

SEQ ID NO: 153 - DNA, 321 bp, Artificial Sequence; identical to SEQ ID NO: 29
  Description of Artificial Sequence Synthetic polynucleotide

SEQ ID NO: 154 - DNA, 450 bp, Artificial Sequence; identical to SEQ ID NO: 30
  Description of Artificial Sequence Synthetic polynucleotide

SEQ ID NO: 155 - DNA, 330 bp, Artificial Sequence; identical to SEQ ID NO: 31
  Description of Artificial Sequence Synthetic polynucleotide

SEQ ID NO: 156 - DNA, 432 bp, Artificial Sequence; identical to SEQ ID NO: 32
  Description of Artificial Sequence Synthetic polynucleotide

SEQ ID NO: 157 - DNA, 324 bp, Artificial Sequence; identical to SEQ ID NO: 33
  Description of Artificial Sequence Synthetic polynucleotide

SEQ ID NO: 158 - DNA, 360 bp, Artificial Sequence; identical to SEQ ID NO: 34
  Description of Artificial Sequence Synthetic polynucleotide

SEQ ID NO: 159 - DNA, 309 bp, Artificial Sequence; identical to SEQ ID NO: 35
  Description of Artificial Sequence Synthetic polynucleotide

SEQ ID NO: 160 - DNA, 372 bp, Artificial Sequence; identical to SEQ ID NO: 36
  Description of Artificial Sequence Synthetic polynucleotide

SEQ ID NO: 161 - DNA, 324 bp, Artificial Sequence; identical to SEQ ID NO: 37
  Description of Artificial Sequence Synthetic polynucleotide

SEQ ID NO: 162 - DNA, 357 bp, Artificial Sequence; identical to SEQ ID NO: 38
  Description of Artificial Sequence Synthetic polynucleotide

SEQ ID NO: 163 - DNA, 324 bp, Artificial Sequence; identical to SEQ ID NO: 39
  Description of Artificial Sequence Synthetic polynucleotide

SEQ ID NO: 164 - protein, 123 aa, Artificial Sequence; identical to SEQ ID NO: 40
  Description of Artificial Sequence Synthetic polypeptide

SEQ ID NO: 165 - protein, 108 aa, Artificial Sequence; identical to SEQ ID NO: 41
  Description of Artificial Sequence Synthetic polypeptide

SEQ ID NO: 166 - protein, 118 aa, Artificial Sequence
  Description of Artificial Sequence Synthetic polypeptide
QVQLVQSGAEVKKPGATVKISCKVSGYTFSDLYMHWVQQAPGNGLEWMGFVDPEDGETIYAEKFQGRLTITADTSTDTAYMELSSLRSEDTAVYYCATGLLGEAFDIWGQGTMVTVSS

SEQ ID NO: 167 - protein, 111 aa, Artificial Sequence; identical to SEQ ID NO: 43
  Description of Artificial Sequence Synthetic polypeptide

SEQ ID NO: 168 - protein, 117 aa, Artificial Sequence
  Description of Artificial Sequence Synthetic polypeptide
EVQLVESGGGLVQPGGSLRLSCAASGITVSTTYMSWVRQAPGKGLDWVSVIYSDGSTHYADSVKGRFTISRDNSKNTLFLQMNSLRAEDTAVYYCASQWTAYCNFRWGQGTLVTVSS

SEQ ID NO: 169 - protein, 111 aa, Artificial Sequence; identical to SEQ ID NO: 45
  Description of Artificial Sequence Synthetic polypeptide

SEQ ID NO: 170 - protein, 119 aa, Artificial Sequence; identical to SEQ ID NO: 46
  Description of Artificial Sequence Synthetic polypeptide

SEQ ID NO: 171 - protein, 131 aa, Artificial Sequence; identical to SEQ ID NO: 47
  Description of Artificial Sequence Synthetic polypeptide

SEQ ID NO: 172 - protein, 119 aa, Artificial Sequence
  Description of Artificial Sequence Synthetic polypeptide
EVQLVESGGGLVQPGGSLRLSCAASGFTFSNNAMSWVRQTPGKGLEWVSSFSGGRDTTYYADSVKGRFTISRDDSKNTLFLQMSSLRAEDTAVYYCAKDLGLLRGIANWGQGTLVTVSS

SEQ ID NO: 173 - protein, 110 aa, Artificial Sequence; identical to SEQ ID NO: 49
  Description of Artificial Sequence Synthetic polypeptide

SEQ ID NO: 174 - protein, 146 aa, Artificial Sequence; identical to SEQ ID NO: 50
  Description of Artificial Sequence Synthetic polypeptide

SEQ ID NO: 175 - protein, 127 aa, Artificial Sequence
  Description of Artificial Sequence Synthetic polypeptide
KLTMETDTLLLWVLLLWVPGSTGDEIVLTQSPGTLSLSPGERATLSCRASQSVSSRSLAWYQQKPGQAPRLLIYGASSRATGIPDRFSGSGSGTDFTLTISRLEPEDFAVYYCQQSTTFGGGTKVEI

SEQ ID NO: 176 - protein, 120 aa, Artificial Sequence; identical to SEQ ID NO: 52
  Description of Artificial Sequence Synthetic polypeptide

SEQ ID NO: 177 - protein, 107 aa, Artificial Sequence; identical to SEQ ID NO: 53
  Description of Artificial Sequence Synthetic polypeptide

SEQ ID NO: 178 - protein, 127 aa, Artificial Sequence; identical to SEQ ID NO: 54
  Description of Artificial Sequence Synthetic polypeptide

SEQ ID NO: 179 - protein, 112 aa, Artificial Sequence; identical to SEQ ID NO: 55
  Description of Artificial Sequence Synthetic polypeptide

SEQ ID NO: 180 - protein, 120 aa, Artificial Sequence
  Description of Artificial Sequence Synthetic polypeptide
QVQLQESGAGLLKPSETLSLTCAVYGGSFSGYYWSWIRQPPGKGLEWIGEIIHSGSTNYNPSLKSRVTISVDTSNNQFSLKLSSVTAADTAVYYCARGRRLLWFGDFDYWGQGTLVTVSS

SEQ ID NO: 181 - protein, 107 aa, Artificial Sequence
  Description of Artificial Sequence Synthetic polypeptide
EIVLTQSPGTLSLSPGERATLSCRASQSVSGSYLAWYQQKPGQAPRLLIYGASSRATGIPDRFNGSGSGTDFTLTISRLEPEDFAVYYCQQYGSSPAFGQGTKVEIK

SEQ ID NO: 182 - protein, 120 aa, Artificial Sequence; identical to SEQ ID NO: 58
  Description of Artificial Sequence Synthetic polypeptide

SEQ ID NO: 183 - protein, 109 aa, Artificial Sequence; identical to SEQ ID NO: 59
  Description of Artificial Sequence Synthetic polypeptide

SEQ ID NO: 184 - protein, 123 aa, Artificial Sequence; identical to SEQ ID NO: 60
  Description of Artificial Sequence Synthetic polypeptide

SEQ ID NO: 185 - protein, 110 aa, Artificial Sequence; identical to SEQ ID NO: 61
  Description of Artificial Sequence Synthetic polypeptide

SEQ ID NO: 186 - protein, 121 aa, Artificial Sequence
  Description of Artificial Sequence Synthetic polypeptide
QVQLVQSGPEVKKPGTSMKISCKASGFTFTRSTMQWVRQARGQRLEWIGWIVVGSGNTNYAQKFQERVTITRDMSTSTAYMELSSLRSEDTAVYYCAAAPVGPTSNWFDPWGQGTLVTVSS

SEQ ID NO: 187 - protein, 107 aa, Artificial Sequence
  Description of Artificial Sequence Synthetic polypeptide
DIQMTQSPSSLSASVGDRVTITCRASQSIINYLNWYQQKPGRAPKLLIYAASSLLSGVPSRFSGSGSGTDFTLTISSLQPEDFATYYCQQSYSTPYTFGQGTKLEIK

SEQ ID NO: 188 - protein, 122 aa, Artificial Sequence
  Description of Artificial Sequence Synthetic polypeptide
QVQLVQSGAEVKKPGASVKVSCKASGYIFISYFMHWVRQAPGQGLEWMGIINPSSGDTRYAQKFQGRVTMTRDTSTNTVYMELSSLRSDDTAVYYCARRPGGLERHNWLDPWGQGTLVTVSS

SEQ ID NO: 189 - protein, 108 aa, Artificial Sequence
  Description of Artificial Sequence Synthetic polypeptide
EIVMTQSPATLSVSPGERATLSCRASQSISSNLAWYQQKPGQAPRLLIYGASTRATGTPARFSGSGSGTEFTLTISSLQSEDFASYYCQQYNNWPAITFGQGTRLEIK

SEQ ID NO: 190 - protein, 119 aa, Artificial Sequence; identical to SEQ ID NO: 66
  Description of Artificial Sequence Synthetic polypeptide

SEQ ID NO: 191 - protein, 107 aa, Artificial Sequence; identical to SEQ ID NO: 67
  Description of Artificial Sequence Synthetic polypeptide

SEQ ID NO: 192 - protein, 150 aa, Artificial Sequence; identical to SEQ ID NO: 68
  Description of Artificial Sequence Synthetic polypeptide

SEQ ID NO: 193 - protein, 110 aa, Artificial Sequence; identical to SEQ ID NO: 69
  Description of Artificial Sequence Synthetic polypeptide

SEQ ID NO: 194 - protein, 144 aa, Artificial Sequence; identical to SEQ ID NO: 70
  Description of Artificial Sequence Synthetic polypeptide

SEQ ID NO: 195 - protein, 108 aa, Artificial Sequence; identical to SEQ ID NO: 71
  Description of Artificial Sequence Synthetic polypeptide

SEQ ID NO: 196 - protein, 120 aa, Artificial Sequence; identical to SEQ ID NO: 72
  Description of Artificial Sequence Synthetic polypeptide

SEQ ID NO: 197 - protein, 103 aa, Artificial Sequence; identical to SEQ ID NO: 73
  Description of Artificial Sequence Synthetic polypeptide

SEQ ID NO: 198 - protein, 124 aa, Artificial Sequence; identical to SEQ ID NO: 74
  Description of Artificial Sequence Synthetic polypeptide

SEQ ID NO: 199 - protein, 108 aa, Artificial Sequence; identical to SEQ ID NO: 75
  Description of Artificial Sequence Synthetic polypeptide

SEQ ID NO: 200 - protein, 119 aa, Artificial Sequence; identical to SEQ ID NO: 76
  Description of Artificial Sequence Synthetic polypeptide

SEQ ID NO: 201 - protein, 108 aa, Artificial Sequence; identical to SEQ ID NO: 77
  Description of Artificial Sequence Synthetic polypeptide

SEQ ID NO: 202 - DNA, 364 bp, Artificial Sequence
  Description of Artificial Sequence Synthetic polynucleotide
gaggtgcagctggtggagtctgggggagacttggtacagcctggcaggtccctgagactctcctgtgcagcctctggattcacctttgatgattatgccatgcactgggtccggcaagctccagggaagggcctggagtggatctcaggtattagtgggaatagtggtagcagaggctatgcggactctgtgaagggccgattcaccatctccagagacaacgccaagaactccctgtatctgcaaatgaacagtctgagagctgaggacacggccttgtattactgtgcaacagatggagacagatctggcactacgcctcttgaccattggggccagggaacccgggtcaccgtctcctcag

SEQ ID NO: 203 - DNA, 322 bp, Artificial Sequence
  Description of Artificial Sequence Synthetic polynucleotide
tcctatgagctgacacagccaccctcggtgtcagtgtccccaggacaaacggccaggatcacctgctctggagatgcattgccaaaaaattatgcttattggtaccagcagaagtcaggccaggcccctgcgttggtcatctatgaggacagcaaacgaccctccgggatccctgagagattctctggctccagctcagggacaatggccaccttgactatcagtggggcccaggtggaggatgaagctgactactactgttactcaacagacagcagtggtactccctccttcggaactgggaccaaggtcaccgtcctag

SEQ ID NO: 204 - DNA, 361 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagttatgccatgaggtgggtccgccaggctccagggaaggggctggagtgggtctcaggtattagtggtagtggtggtagcacatactacgcagactccgtgaagggccggttcaccatctccagagacaattccaagatcacgctgtatctgcaaatggacagcctgagagtcgaggacacggccgtatattactgtgcgacagcgcccctcagctatgattcctccacggactactggggccagggaaccctggtcaccgtctcctcag

SEQ ID NO: 205 - DNA, 322 bp, Artificial Sequence
  Description of Artificial Sequence Synthetic polynucleotide
tcctatgagctgacacagccaccctcggtgtcagtgtccccaggacaaacggccaggatcacctgctctggagatgcattgccaaaaaaatatgcttattggtaccagcagaagtcaggccaggcccctgtgctggtcatctatgaggacagcaaacgaccctccgggatccctgagagattctctggctccagctcagggacaatggccaccttgactatcagtggggcccaggtggaggatgaagctgactactactgttactcaacagacagcagtggtaatccccgattcggcagagggaccaagctgaccgtcctag

SEQ ID NO: 206 - DNA, 363 bp, Artificial Sequence; identical to SEQ ID NO: 100
  Description of Artificial Sequence Synthetic polynucleotide

SEQ ID NO: 207 - DNA, 321 bp, Artificial Sequence; identical to SEQ ID NO: 101
  Description of Artificial Sequence Synthetic polynucleotide

SEQ ID NO: 208 - protein, 121 aa, Artificial Sequence
  Description of Artificial Sequence Synthetic polypeptide
EVQLVESGGDLVQPGRSLRLSCAASGFTFDDYAMHWVRQAPGKGLEWISGISGNSGSRGYADSVKGRFTISRDNAKNSLYLQMNSLRAEDTALYYCATDGDRSGTTPLDHWGQGTRVTVSS

SEQ ID NO: 209 - protein, 107 aa, Artificial Sequence
  Description of Artificial Sequence Synthetic polypeptide
SYELTQPPSVSVSPGQTARITCSGDALPKNYAYWYQQKSGQAPALVIYEDSKRPSGIPERFSGSSSGTMATLTISGAQVEDEADYYCYSTDSSGTPSFGTGTKVTVL

SEQ ID NO: 210 - protein, 120 aa, Artificial Sequence
  Description of Artificial Sequence Synthetic polypeptide
EVQLVESGGGLVQPGGSLRLSCAASGFTFSSYAMRWVRQAPGKGLEWVSGISGSGGSTYYADSVKGRFTISRDNSKITLYLQMDSLRVEDTAVYYCATAPLSYDSSTDYWGQGTLVTVSS

SEQ ID NO: 211 - protein, 107 aa, Artificial Sequence
  Description of Artificial Sequence Synthetic polypeptide
SYELTQPPSVSVSPGQTARITCSGDALPKKYAYWYQQKSGQAPVLVIYEDSKRPSGIPERFSGSSSGTMATLTISGAQVEDEADYYCYSTDSSGNPRFGRGTKLTVL

SEQ ID NO: 212 - protein, 121 aa, Artificial Sequence; identical to SEQ ID NO: 118
  Description of Artificial Sequence Synthetic polypeptide

SEQ ID NO: 213 - protein, 107 aa, Artificial Sequence; identical to SEQ ID NO: 119
  Description of Artificial Sequence Synthetic polypeptide

(19) United States
(12) Patent Application Publication
(10) Pub. No.: US 2015/0239961 A1
(43) Pub. Date: Aug. 27, 2015

(54) ADCC-MEDIATING ANTIBODIES, COMBINATIONS AND USES THEREOF

(71) Applicants: DUKE UNIVERSITY, Durham, NC (US); THE GOVERNMENTOF THE UNITED STATES, as represented by THE SECRETARY OF THE ARMY, on behalf of WALTER, Fort Detrick, MD (US)

(72) Inventors: Barton F. Haynes, Durham, NC (US); Mattia Bonsignori, Durham, NC (US); Hua-xin Liao, Durham, NC (US); Guido Ferrari, Durham, NC (US); Michael A. Moody, Durham, NC (US); Jerome Kim, Fort Detrick, MD (US); Nelson Michael, Fort Detrick, MD (US); Justin Pollara, Durham, NC (US)

(21) Appl. No.: 14/431,537

(22) PCT Filed: Sep. 26, 2013
(86) PCT No.: PCT/US2013/061963

§ 371 Date: Mar. 26, 2015

Related U.S. Application Data
(60) Provisional application No. 61/705,922, filed on Sep. 26, 2012.
(60) Provisional application No. 61/762,543, filed on Feb. 8, 2013.

(51) Int. Cl. C07K 16/10

(52) U.S. Cl. CPC C07K 16/1063; C07K 2317/732; C07K 2317/76

(57) ABSTRACT

The present invention relates, in general, to antibody-dependent cellular cytoxicity (ADCC)-mediating antibodies, and, in particular, to ADCC-mediating antibodies (and fragments thereof) suitable for use, for example, in reducing the risk of HIV-1 infection in a subject. The invention further relates to compositions comprising such antibodies or antibody fragments.

This application claims priority to U.S. Prov. Appln. Ser. No. 61/705,922 filed Sep. 26, 2012 and U.S. Prov Appln. Ser. No. 61/762,543 filed Feb. 8, 2013. The content of each application is hereby incorporated by reference in its entirety.

This invention was made with government support under Grant Number Um1-AI100645 awarded by the National Institutes of Health, CHAVI (U 19 AI067854), National Institutes of Health (NIH/NIAID/DAIDS) and Bill and Melinda Gates Foundation Grants (OPP1033098). The government has certain rights in the invention.

### TECHNICAL FIELD

The present invention relates, in general, to antibody-dependent cellular cytoxicity (ADCC)-mediating antibodies, and, in particular, to ADCC-mediating antibodies suitable for use, for example, in reducing the risk of HIV-1 infection in a subject. The invention further relates to compositions comprising such antibodies.

### BACKGROUND

The RV144 ALVAC-HIV (vCP1521) prime/AIDSVAX B/E boost clinical trial provided the first evidence of vaccine-induced protection from acquisition of HIV-1 infection (Rerks-Ngarm et al, N. Engl. J. Med. 361:2209-2220 (2009)). Analysis of immune correlates of risk of infection demonstrated that antibodies targeting the Env gp120 V1/V2 region inversely correlated with infection risk, while IgA Env-binding antibodies to Env directly correlated with infection risk (Haynes, Case-control study of the RV144 trial for immune correlates: the analysis and way forward, abstr., p. AIDS Vaccine Conference, Bangkok, Thailand, Sep. 12-15, 2011, Haynes et al, N Engl J Med 366(14):1257-86) (2012)). In addition, in secondary immune correlates analyses, low plasma IgA Env antibody levels in association with high levels of ADCC were inversely correlated with infection risk (Haynes, Case-control study of the RV144 trial for immune correlates: the analysis and way forward, abstr., p. AIDS Vaccine Conference, Bangkok, Thailand, Sep. 12-15, 2011, Haynes et al, N Engl J Med 366(14):1257-86) (2012))). Thus, one hypothesis is that the observed protection in RV144 may be due, in a subset of vaccines, to ADCC-mediating antibodies.

The importance of ADCC responses has been reported in chronically HIV-1 infected individuals (Baum et al, J. Immunol. 157:2168-2173 (1996), Ferrari et al, J. Virol. 85:7029-7036 (2011), Lambotte et al, Aids 23:897-906 (2009)), and in HIV-1 vaccine studies in non-human primates (Flores et al, J. Immunol. 182:3718-3727 (2009), Gómez-Rom\acute{α}n et al, J. Immunol. 174:2185-2169 (2005), Hidajat et al, J. Virol. 83:791-801 (2009), Sun et al, J. Virol. 85:6906-6912 (2011)). Baum et al. reported an inverse correlation between titers of HIV-1 gp120-specific ADCC antibodies and the rate of disease progression in humans (Baum et al, J. Immunol. 157:2168-2173 (1996)). Moreover, HIV-1-infected elite controllers who had undetectable viremia showed higher ADCC antibody titers than infected individuals with viremia (Lambotte et al, Aids 23:897-906 (2009)). In non-human primates, administration of vaccine candidates elicited ADCC antibody titers that correlated with control of virus replication after mucosal challenge with a pathogenic SIV (Barouch et al, Nature 482:89-93 (2012), Gómez-Rom\acute{α}n et al, J. Immunol. 174:2185-2169 (2005)). More recently, different groups have reported that titers of non-neutralizing ADCC antibodies are associated with control of viremia against primary SIV infection (Flores et al, J. Immunol. 182:3718-3727 (2009), Hidajat et al, J. Virol. 83:791-801 (2009), Sun et al, J. Virol. 85:6906-6912 (2011)). While antibodies against multiple epitopes can mediate ADCC, it has been recently reported that the A32 mAb, recognizing a conformational epitope in the C1 region of HIV-1 Env gp120 (Wyatt et al, J. Virol. 69:5723-5733 (1995)), could mediate potent ADCC activity and could block a significant proportion of ADCC-mediating Ab activity detectable in HIV-1 infected individuals (Ferrari et al, J. Virol. 85:7029-7036 (2011)).

It has recently been observed that ADCC-mediating Ab responses are detectable as early as 48 days after acute HIV-1 infection (Pollara et al, AIDS Res. Hum. Retroviruses 26: A-12 (2010)). This early appearance of ADCC-mediating Abs after acute HIV-1 infection contrasts with HIV-1 broadly neutralizing antibodies (bNAbs) that appear approximately 2-4 years after HIV-1 infection (Gray et al, J. Virol. 85:7719-7729 (2011), Mikell et al, PLoS Pathog. 7:e1001251 (2011), Shen et al, J. Virol. 83:3617-3625 (2010)).

The present invention is based, at least in part, on studies that resulted in the identification of a series of modestly somatically mutated ADCC-mediating antibodies induced by the ALVAC-HIV/AIDSVAX B/E vaccine (Nitayaphan et al, J. Infect. Dis. 190:702-706 (2004), Rerks-Ngarm et al, N. Engl. J. Med. 361:2209-2220 (2009)), most of which are directed against conformational A32-blockable epitopes of the gp120 envelope glycoprotein. This group of antibodies displayed preferential usage of the variable heavy [VH]1 gene segment, a phenomenon similar to that recently described for highly mutated CD4 binding-site [CD4bs]-specific bNAbs (Scheid et al, Science 333:1633-1637 (2011), Wu et al, Science 333(6049):1593 (2011). Epub 2011 Aug. 11).

### SUMMARY OF THE INVENTION

In general, the invention relates to ADCC-mediating antibodies. More specifically, the invention relates to ADCC-mediating antibodies (and fragments thereof) suitable for use, for example, in reducing the risk of HIV-1 infection in a subject (e.g., a human subject), and to compositions comprising same.

The RV144 HIV-1 vaccine clinical trial showed an estimated vaccine efficacy of 31.2%. Viral genetic analysis identified a vaccine-induced site of immune pressure in the HIV-1 envelope (Env) variable region 2 (V2) focused on residue 169. This residue is included in the epitope recognized by vaccinee-derived CH58 and CH59 V2 monoclonal antibodies (mAbs). Moreover, CH58 binds to the clade B gp70V1/V2 CaseA2 fusion protein used to identify the immune correlates of infection risk and represents one type of antibody associated with lower rate of transmission in the trial. While the RV144 vaccine did not induce antibody responses that neutralize transmitted/founder breakthrough viruses, antibody dependent cellular cytotoxicity (ADCC) antibodies were induced against Env V2 and constant 1 (C1) regions. In this study we demonstrate that C1 and V2 mAbs synergize for binding to Env expressed on the surface of virus-infected CD4+ T cells. Importantly, this antibody interaction increased the HIV-1 ADCC activity of anti-V2 mAb CH58 at concentrations similar to that observed in plasma of RV144 vaccine recipients. These findings demonstrate that vaccine induced anti-Env Ab responses against V2 and C1 specificities synergize in their anti-viral activities, and raise the hypothesis that V2 antibody-mediated reduction in transmission risk may have been associated with C1-V2 antibody synergy.

In certain aspects, the invention provides compositions comprising an isolated anti-V2 (HIV-1 envelope V2) antibody and/or an isolated anti-C1 (HIV-1 envelope C1) antibody. In certain embodiments the antibody is monoclonal. In certain embodiments, the composition is a pharmaceutical composition comprising a pharmaceutically acceptable carrier. In certain embodiments, the composition is consisting essentially of an isolated anti-V2 (HIV-1 envelope V2) antibody and/or an isolated anti-C1 (HIV-1 envelope C1) antibody, fragments, or an antibody comprising sequences as described herein. In certain embodiments, the composition comprises at least one anti-V2 (HIV-1 envelope V2) antibody and/or at least one anti-C1 (HIV-1 envelope C1) antibody. In certain embodiments, the composition comprises two, three or more anti-V2 (HIV-1 envelope V2) antibodies and/or two, three or more anti-C1 (HIV-1 envelope C1) antibodies, or fragments thereof wherein the composition synergistically mediates antibody dependent cellular cytotoxicity. In certain aspects, the invention provides a composition comprising an anti-V2 (HIV-1 envelope V2) antibody fragment comprising an antigen binding portion thereof and an anti-C1 (HIV-1 envelope C1) antibody fragment comprising an antigen binding portion thereof.

In certain embodiments, the compositions mediate HIV-1 anti-viral activity, for example but not limited to virus neutralization, or antibody dependent cellular cytotoxicity. In certain embodiments, the compositions synergistically mediate HIV-1 anti-viral activity, for example but not limited virus neutralization, or antibody dependent cellular cytotoxicity.

In certain embodiments, the anti-V2 antibody comprises a variable heavy chain or a variable light chain from any one of the anti-V2 antibodies described herein. In certain embodiments, the anti-V2 antibody comprises a CDR from any one of the anti-V2 antibodies described herein. In a non-limiting embodiment, the anti-V2 antibody is CH58.

In certain embodiments, the anti-C1 antibody comprises a variable heavy chain or a variable light chain from any one of the anti-C1 antibodies described herein. In certain embodiments, the anti-C1 antibody comprises a CDR from any one of the anti-C1 antibodies described herein. In a non-limiting embodiment, the anti-C1 antibody is CH90.

In certain embodiments, the composition comprises an antibody comprising a variable heavy or a variable light chain, or a CDR from CH58, CH59, HG107, or HG120, and/or an antibody comprising a variable heavy or a variable light chain, or a CDR from CH54, CH57, or CH90. In certain embodiments, the composition comprises CH58 and CH90; HG120 and CH54, CH57, or CH90; CH59 and CH54, or CH57; HG107 and CH90.

In certain embodiments, the antibody is recombinantly produced, or purified from B-cell cultures.

In certain aspects, the invention provides isolated antibodies or fragments thereof, the amino acid sequences of these antibodies or fragments, nucleic acid sequences encoding these antibodies, their variable heavy and light chains, and CDRs.

In certain aspects, the invention provides an isolated monoclonal anti-V2 (HIV-1 envelope V2) antibody or fragment thereof having the binding specificity of any one of antibodies CH58, CH59, HG107, or HG120. In certain aspects, the invention provides an isolated monoclonal anti-V2 antibody or fragment thereof comprising a variable heavy or light chain, or a CDR from any one of antibodies CH58, CH59, HG107, or HG120.

In certain aspects, the invention provides an isolated monoclonal anti-C1 (HIV-1 envelope C1) antibody or fragment thereof having the binding specificity of any one of antibodies CH54, CH57, or CH90. In certain aspects, the invention provides an isolated monoclonal anti-C1 antibody or fragment thereof comprising a variable heavy or light chain, or a CDR from any one of antibodies CH54, CH57, or CH90.

In certain aspects, the invention provides a complementary nucleic acid (cDNA) molecule encoding a variable heavy or light chain from an anti-V2 (HIV-1 envelope V2) antibody or an antigen binding fragment thereof.

In certain aspects, the invention provides a complementary nucleic acid (cDNA) molecule encoding a variable heavy or light chain from an anti-C1 (HIV-1 envelope C1) antibody or an antigen binding fragment thereof.

In certain aspects, the invention provides a vector comprising theses cDNAs.

In certain aspects, the invention provides a host cell comprising the vectors or cDNAs encoding the antibodies of the invention or fragments thereof. Any suitable cell for the expression of the human antibodies of the invention is contemplated. A non-limiting example is a CHO cell line.

In certain aspects, the invention provides a polypeptide comprising the amino acid sequence of an anti-V2 (HIV-1 envelope V2) antibody or an antigen binding fragment thereof. In certain aspects, the invention provides a polypeptide comprising the amino acid sequence of an anti-C1 (HIV-1 envelope C1) antibody or an antigen binding fragment thereof. In certain aspects, the invention provides polypeptide comprising the amino acid sequence or a fragment thereof of any one of the antibodies described herein.

In certain aspects, the invention provides methods of using the inventive antibodies and compositions in immunotherapy regimens, for example but not limited to passive prophylactic or treatment methods. In certain aspects, the invention provides an HIV-1 prophylactic or therapeutic method comprising administering to a subject an antibody composition as described herein in an amount sufficient to reduce the risk or prevent an HIV-1 infection. In certain embodiments, the antibody compositions of the invention reduce the risk of an HIV-1 infection in a subject after administering to the subject a composition as described herein in an amount sufficient to reduce the likelihood of an HIV-1 infection. In certain aspects, the invention provides prophylactic or therapeutic uses of the synergistic antibody compositions of the invention. The compositions of the invention can be further analyzed for their prophylactic, protective and/or therapeutic properties in any suitable models, for example but not limited to a non-human primate model. Objects and advantages of the present invention will be clear from the description that follows.

### BRIEF DESCRIPTION OF THE DRAWINGS

FIGS. 1A-1D. Vaccine-induced ADCC responses. To measure plasma ADCC activity induced by the ALVAC-HIV(vCP1521)/AIDSVAX B/E vaccine, plasma samples from 40 vaccine recipients and 10 placebo recipients were collected before immunization (week 0) and 2 weeks after the last boost (week 26). ADCC activity was measured using the ADCC-CM243 assay (FIGS. 1A-1B) and ADCC-92TH023 assay (FIGS. 1C-1D). Results are reported as Area Under the Curve (AUC). Each dot represents one sample. The lines connect samples obtained from the same donor.

FIGS. 2A and 2B. Recognition of the A32 epitope in plasma of ALVAC-HIV(vCP1521)/AIDSVAX B/E vaccine recipients. (FIG. 2A) Plasma samples collected at week 26 from 20 placebo recipients and 79 vaccine recipients were evaluated for the presence of Abs with A32-like binding specificities by competition ELISA. Plasmas that inhibited >50% of A32 mAb binding were defined as positive (red dots). While none of the placebo recipients displayed A32-like responses, the plasma of 76/79 vaccine recipients (96.2%) competed A32 mAb binding to its cognate epitope. The Whisker boxes show the average plasma ID₅₀ titer, and the 95% confidence interval for each test group. (FIG. 2B) Inhibition of plasma ADCC activity by epitope blocking with mAb A32 Fab fragment was evaluated in the ADCC-CM243 assay. Plasma samples were collected at week 26 from 30 vaccine recipients and were tested at dilutions corresponding to peak activity. Data are reported as maximum % GzB activity detected using CM243-gp120 coated targets without pre-treatment (no Fab pretreatment; left) or treated with 10 μg/mL mAb A32 Fab (center) or Palivizumab Fab (negative control; right). Lines and error bars represent the mean % GzB activity±SD. The P-values were obtained using repeated measure ANOVA.

FIGS. 3A and 3B. ADCC activity of vaccine-induced mAbs. ADCC activity mediated by monoclonal antibodies isolated from memory B cells of ALVAC-HIV(vCP1521)/AIDSVAX B/E vaccine recipients. Twenty-three mAbs were isolated from six vaccine recipients. Each bar is color-coded by subject: T141485 (light blue), T141449 (red), T143859 (brown), 609107 (green), 210884 (orange) and 347759 (dark blue). MAb A32 (positive control) and Palivizumab (negative control) are shown in black and white, respectively. The plots show (FIG. 3A) the maximum percentage of granzyme B activity (Maximum % GzB) with the threshold of positivity (5%) indicated by the black line, and (FIG. 3B) the end-point titer expressed in μg/ml for each mAb. The threshold of positivity was determined by averaging the results obtained by testing over 70 mAbs with different binding capacity to gp120 and infected cells. Shown data refer to the results obtained with the ADCC-CM235 assay with the exception of mAb CH23, for which results of the ADCC-CM243 are shown. ADCC activity of all mAbs was confirmed in the ADCC-CM235 assay with a 6-hour incubation (not shown; Spearman correlation analysis p=0.001).

FIG. 4. Monoclonal Antibody competition of A32, 17B and 19B Fab ADCC activity. The 20 ADCC-mediating mAbs that did not bind to linear epitopes were tested for their ability to inhibit ADCC mediated by Fab A32 (left), 17B (middle) and 19B (right) in the ADCC-E.CM235 assay. The y-axis shows the average of inhibition of ADCC activity in duplicate assays and each bar is color-coded by subject as in FIG. 3.

FIG. 5. Monoclonal Antibody competition of A32 mAb binding to HIV-1 AE.A244 gp120 envelope glycoprotein. The ADCC-mediating mAbs (with the exception of CH55 and CH80) were tested for their ability to compete mAb A32 binding to AE.A244 gp120 envelope glycoprotein. The y-axis shows the percentage of blocking of binding activity and each bar is color-coded by subject as in FIG. 3. The data shown are representative of duplicate independent experiments.

FIG. 6. Cross-clade activity of RV144-induced ADCC-mediating mAbs. Twenty-one mAbs isolated from six vaccine recipients were tested against the E.CM235- (black bar), B.BaL- (red bar), C.DU422 (blue bar), and C.DU151-infected (green bar) CEM.NKR_{CCR5} target cells using the GTL assay. The plot shows the average end-point titer from duplicate values expressed in μg/ml for each mAb and calculated as previously described for FIG. 3.

FIG. 7. VH gene segment usage of the ADCC-mediating monoclonal antibodies. The pie-chart shows the distribution of VH gene segment and allele usage of the 23 ADCC-mediating mAbs. Each antibody is color-coded by subject of origin using the same color scheme as in FIG. 3. The yellow fill indicates all mAbs that used VH1.

FIGS. 8A and 8B. Characteristics of antibodies that used VH1 gene segments. (FIG. 8A) Amino acid sequences of ADCC-mediating antibodies that used VH1 gene segments were aligned to the heavy and light chain consensus HAAD motifs previously identified for CD4bs bNAbs antibodies, which were described to preferentially use the VH1 gene, in particular the VH1-2*02 and 1-46 segments (Scheid et al, Science 333:1633-1637 (2011)). The consensus HAAD motifs of the heavy and light chains are 68 and 53 amino acid-long, respectively. Data are plotted as number of identical amino acids for heavy chain (x-axis) and light chain (y-axis). Black Xs=CD4bs bNAb antibodies (Scheid et al, Science 333:1633-1637 (2011)); red circles=VH1 ADCC mediating antibodies (range 46 to 57/68 as identity for heavy chain, 68-84%; 37 to 46/53 aa identity for light chain, 70-87%); blue diamonds=influenza broadly neutralizing antibodies (49) (52 to 55/68 as identity for heavy chain, 76-81%; 31 to 32/53 aa identity for light chain, 58-60%). (FIG. 8B) Maximal % GzB activity is correlated with HC mutation frequency (Spearman correlation p=0.56, p=0.02). Antibodies that blocked sCD4 binding to gp120 are shown as red diamonds and were found throughout the range of mutation frequencies; those without blocking activity are shown as black circles.

FIG. 9. Heavy and light chain sequences of CH21, CH22, CH23, CH29, CH38, CH40, CH42, CH43, CH51, CH52, CH53, CH54, CH55, CH57, CH58, CH59, CH60, CH73, CH89.

FIG. 10. Nucleotide sequences encoding VH and VL chains of CH20 and A32 antibodies and amino acid sequences of VH and VL chains of CH20 and A32.

FIG. 11. Nucleotide sequences encoding VH and VK chains of 7B2 antibody and amino acid sequences of VH and VK chains of 7B2.

FIG. 12. Nucleotide sequences encoding VH and VL chains of CH49, CH77, CH78, CH81, CH89, CH90, CH91, CH92 and CH94 antibodies and amino acid sequences of VH and VL chains of CH49, CH77, CH78, CH81, CH89, CH90, CH91, CH92 and CH94.

FIG. 13. Synergy of mAb binding to the monomeric gp120 by SPR. A) Schematic of the SPR assay utilized to test the presence of synergy between the anti-V2 and anti-C1 mAb for binding to the recombinant AE.244 Δ11 gp120 according to the procedure reported in the Method section. B) SPR of binding of the CH58 mAb alone or in combination with the other anti-C1 mAbs. The y-axis represents the RUA values and the x-axis the time in milliseconds. C) Fold increase of the anti-V2 CH58 binding to the recombinant AE.A244Δ11 gp120. The data are reported as percent increase calculated based to the binding of the CH58 mAb of gp120 incubated with the murine gp120 16H3 mAb used as negative control.

FIG. 14. Synergy of mAb for binding to the infected CD4 T cells. Primary CD4⁺ T cells were activated and infected with the HIV-1 AE.92TH023 (A-C) and AE.CM235 (D and E) for 72 hours. Cells were stained with viability dye and anti-p24 Ab to identify viable infected cells. The CH58 mAb was conjugated with Alexa Fluor®-488 fluoropohore. The other mAbs and mAb Fab fragments (Palivizumab (Neg), A32, CH54, CH57, and CH90) were used as non conjugated reagents. The gating strategy used for detection of HIV envelope on the surface of infected cells is shown in Panel A. (B-E) The infected CD4⁺ T cells were stained with CH58 Alexa Fluor®-488 in combination with the mAbs or Fab fragments indicated on the x-axes at 10 μg/ml each. The y-axes represent the % increase of stained cells (B and D) and Mean Fluorescent Intensity (MFI; C and E) for each combination of mAb or Fab fragment relative to the staining of cells observed when the CH58 mAbs was used alone.

FIG. 15. Synergy of anti-V2 and anti-C1 mAbs for ADCC. Each graph represent the % Specific Killing observed by incubating individual mAbs and the combinations indicated with HIV-1 AE.CM235-infected CEM.NKR_{CCR5} target cells for 3 hours in the Luciferase ADCC assay. The expected ADCC activity if the combinations result in an additive effect are represented by white bars. The actual observed activities are represented by filled bars. A.) Mean and interquartile ranges of the expected and observed ADCC activities of all tested concentrations of the mAb pairs indicated. B) Expected and observed ADCC activity of anti-C1 CH90 mAb and anti-V2 CH58 mAb for all combinations tested. Results represent the mean and SEM of two independent experiments, each run in duplicate.

FIG. 16. Synergy for ADCC at 1:1 ratio of anti-V2 and anti-C1 mAbs. % Specific Killing observed by anti-V2 mAbs CH58 (A), CH59 (B), HG107 (C) and HG120 (D) alone and in combination with negative control Palivizumab or anti-C1 mAbs CH54, CH57, and CH90 at a 1:1 ratio over 5-fold serial dilutions in the Luciferase ADCC assay with CM235-infected targets. The combination curve is represented by a diamond and is indicated by an arrow.

FIG. 17. Synergy of CH58 anti-V2 IgG and CH90 anti-C1 F(ab′)₂ for ADCC. ADCC synergy observed between CH58 IgG and CH90 F(ab′)₂ against HIV-1 AE.CM235-infected CEM.NKR_{CCR5} target cells. The graph represents the % increase of ADCC activity for the combination of CH90 F(ab′)₂ and CH58 IgG indicated as calculated by comparison to the activity of CH58 alone. CH90 F(ab′)₂ alone was unable to mediate ADCC.

FIG. 18. Synergy for ADCC at 1:1 ratio of anti-V2 and anti-C1 mAbs. A.) % Specific Killing observed by CH58, CH90, and CH58 in combination with CH90 at a 1:1 ratio over 5-fold serial dilutions in the Luciferase ADCC assay with CM235-infected targets. The dashed line represents 75% of the peak activity observed for the V2 mAb CH58 alone (PC75). B) Summary of maximum % killing, endpoint concentration (EC), and PC75 for each mAb alone or in combination. The combination index (CI) values at EC and PC75 are included, and indicate values consistent with synergistic interactions (C1<1) for both mutually exclusive and mutually non-exclusive interactions.

FIG. 19. Heavy and light chain sequences of CH21, CH22, CH23, CH29, CH38, CH40, CH42, CH43, CH5 I, CH52, CH53, CH54, CH55, CH57, CH58, CH59, CH60, CH73, and CH89. Sequences of CDR1, 2, and 3 are underlined.

FIG. 20. Heavy and light chain sequences of HG107, HG120 and CH90. Sequences of CDR1, 2, and 3 are underlined.

### DETAILED DESCRIPTION OF THE INVENTION

A series of modestly somatically mutated ADCC-mediating antibodies induced by the ALVAC-HIV/AIDSVAX B/E vaccine have been identified. Most are directed against conformational A32-blockable epitopes of the gp120 envelope glycoprotein. This group of antibodies displays preferential usage of the variable heavy [VH]1 gene segment, a phenomenon similar to that recently described for highly mutated CD4 binding-site [CD4bs]-specific bNAbs. The present invention relates to such ADCC-mediating antibodies, and fragments thereof, and to the use of same, alone or in combination with therapeutics, in reducing the risk of HIV-1 infection in a subject (e.g., a human), in inhibiting disease progression in infected subjects (e.g., humans) and in eradicating HIV-1-infected cells to cure a person of HIV-1 infection. In one embodiment, the antibodies, or fragments thereof, are used to target toxins to HIV-1 infected cells.

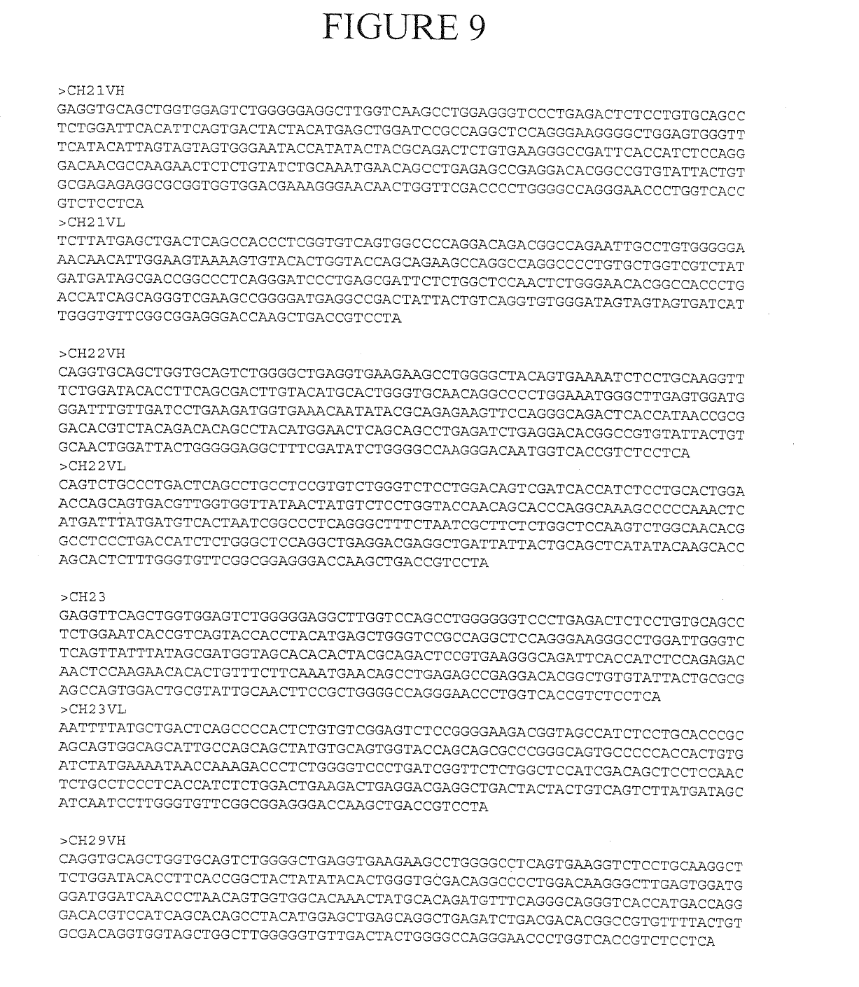

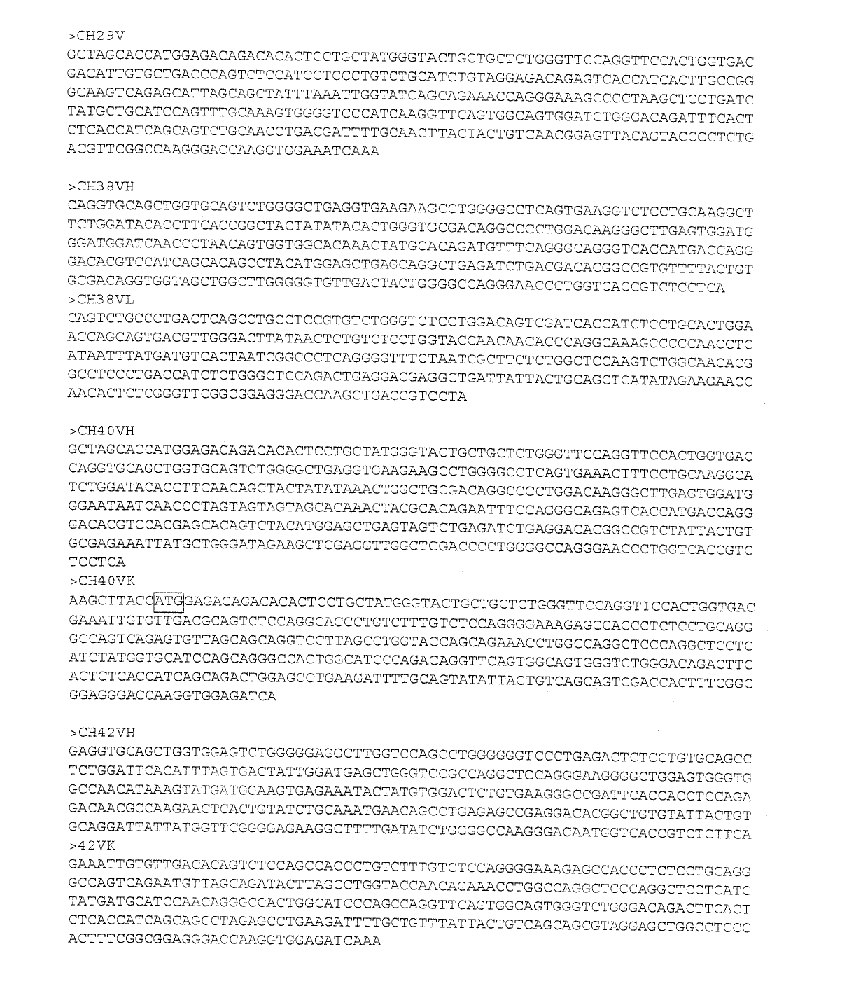

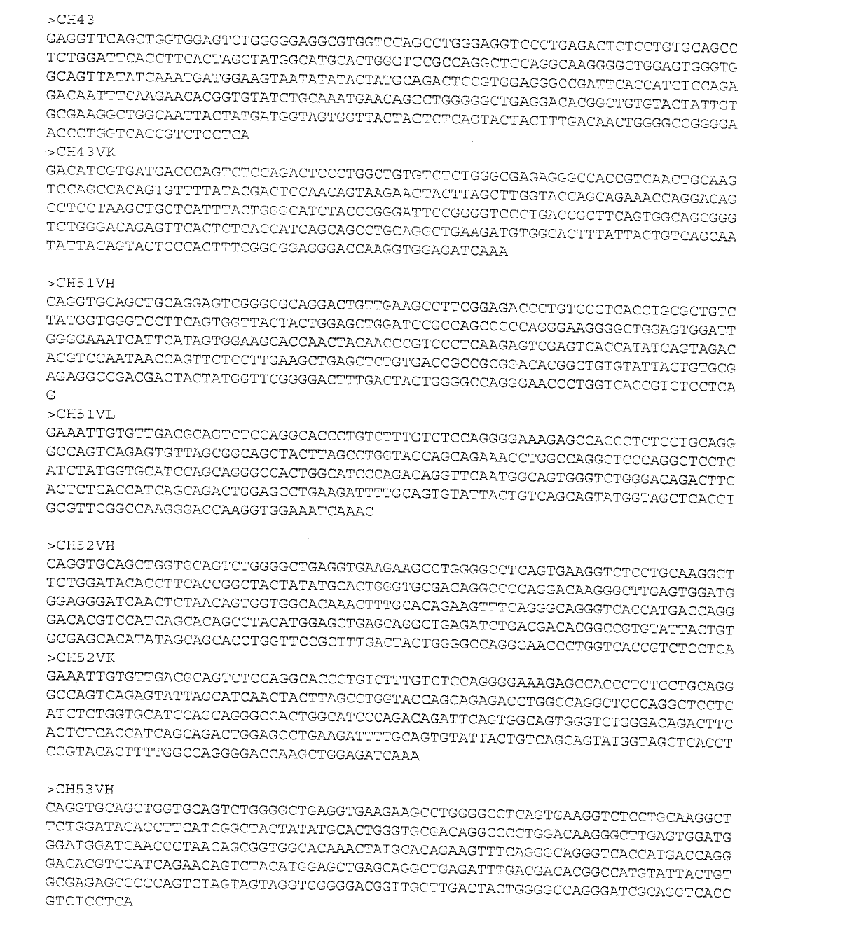

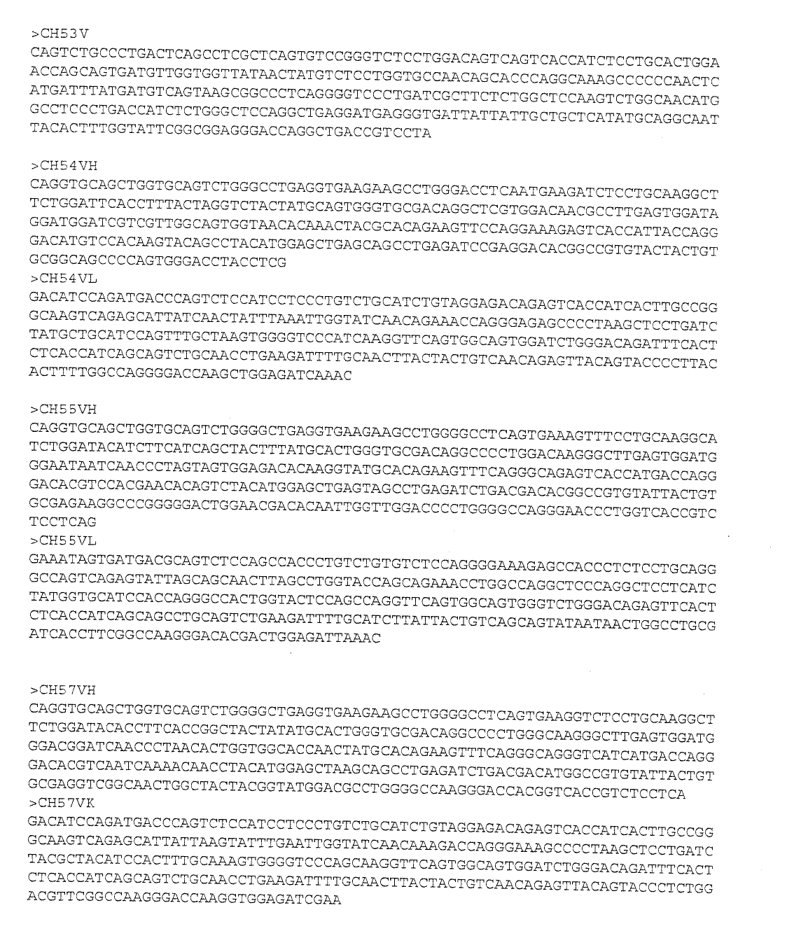

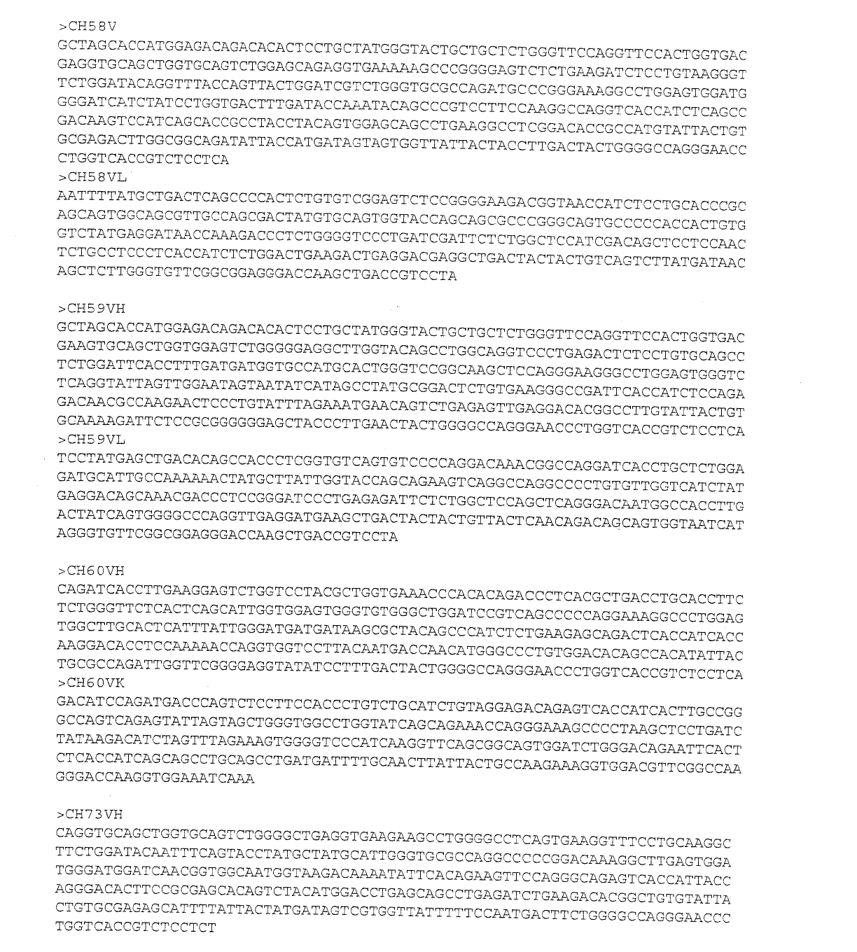

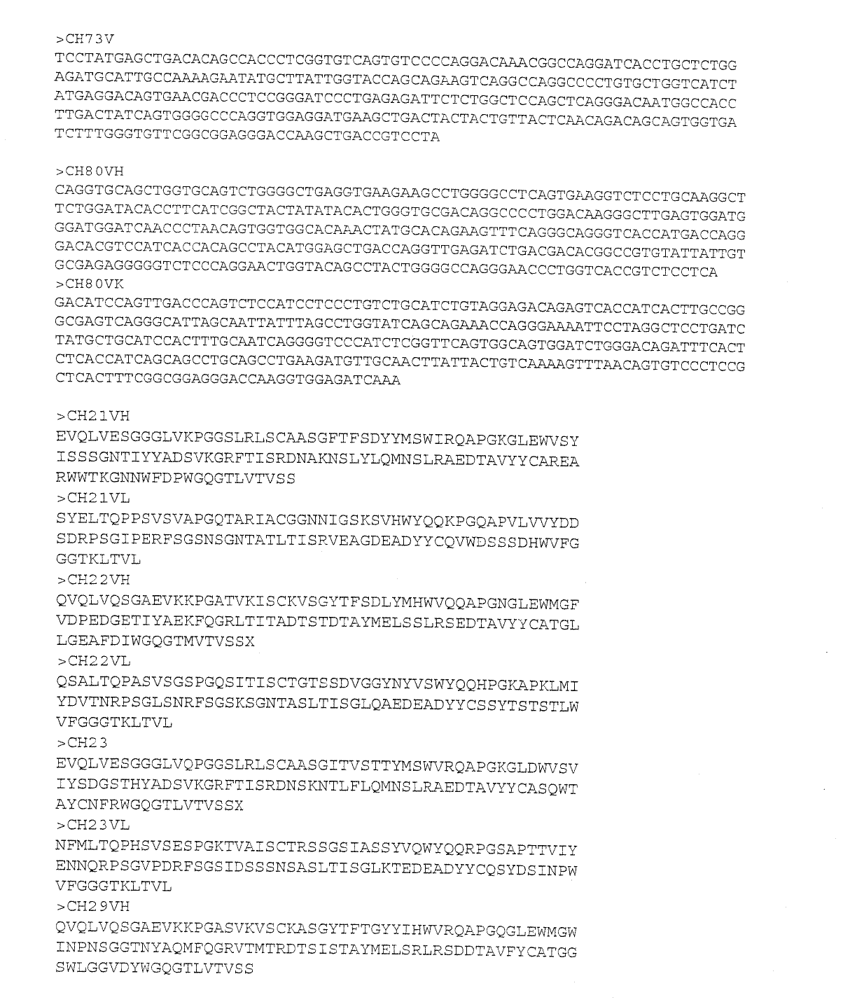

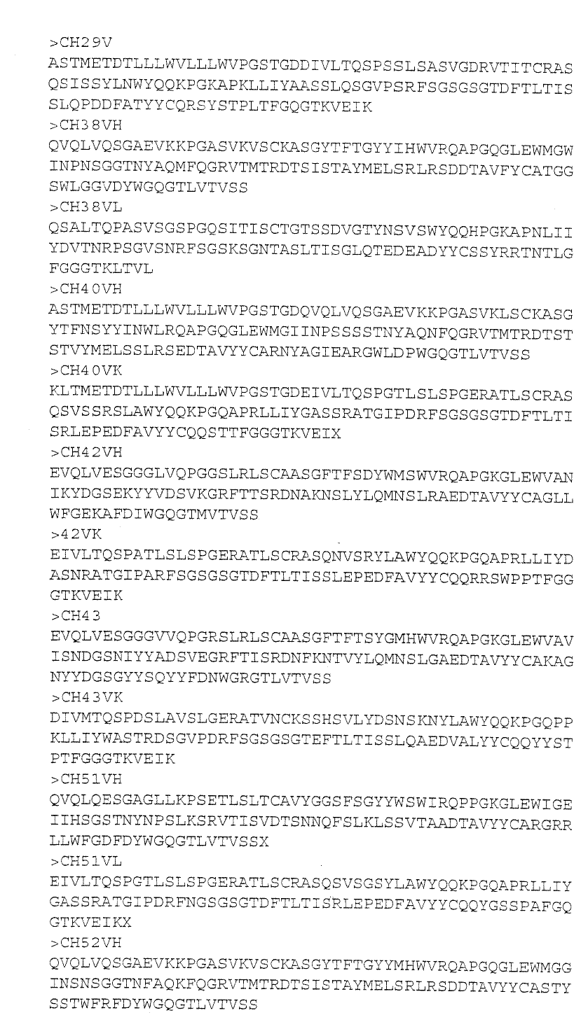

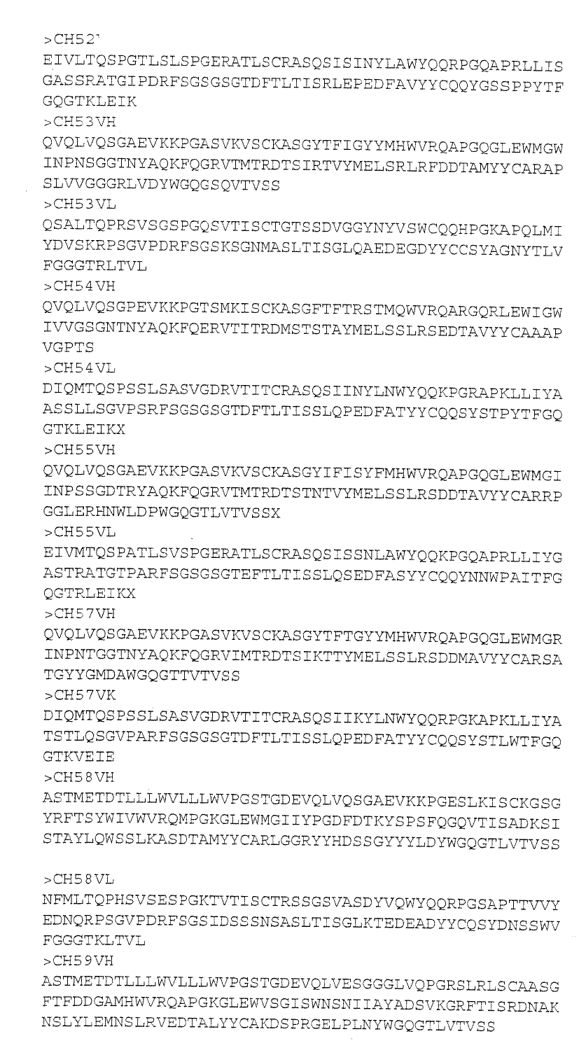

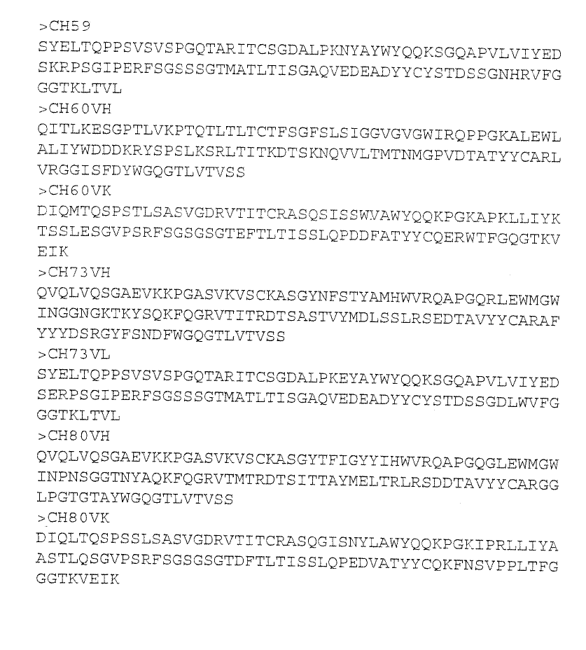

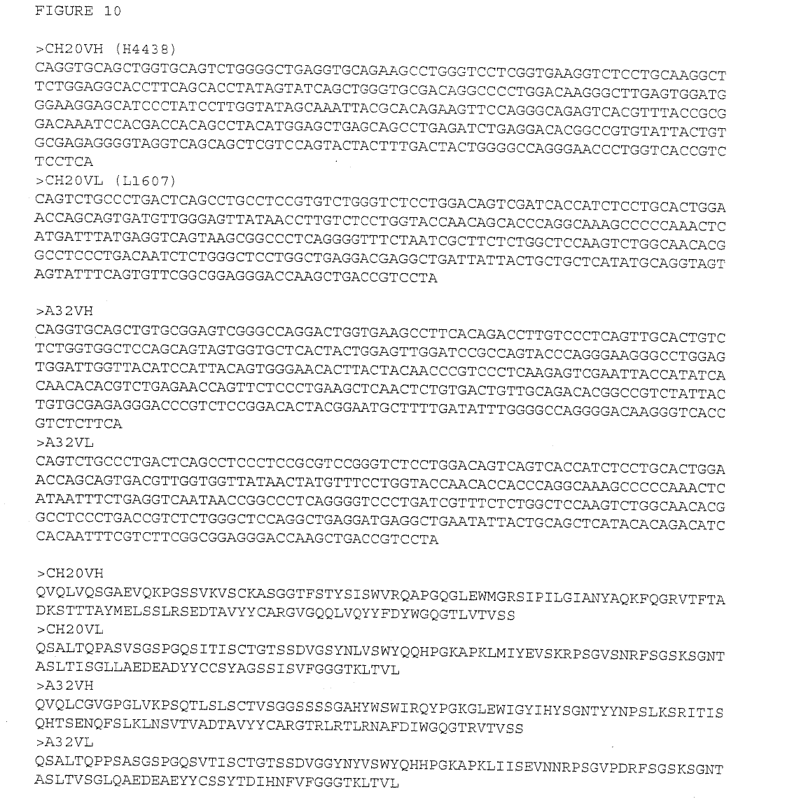

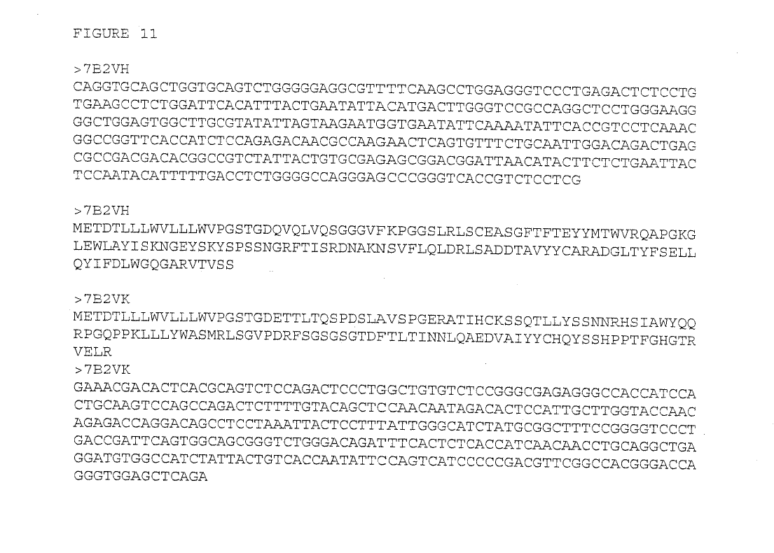

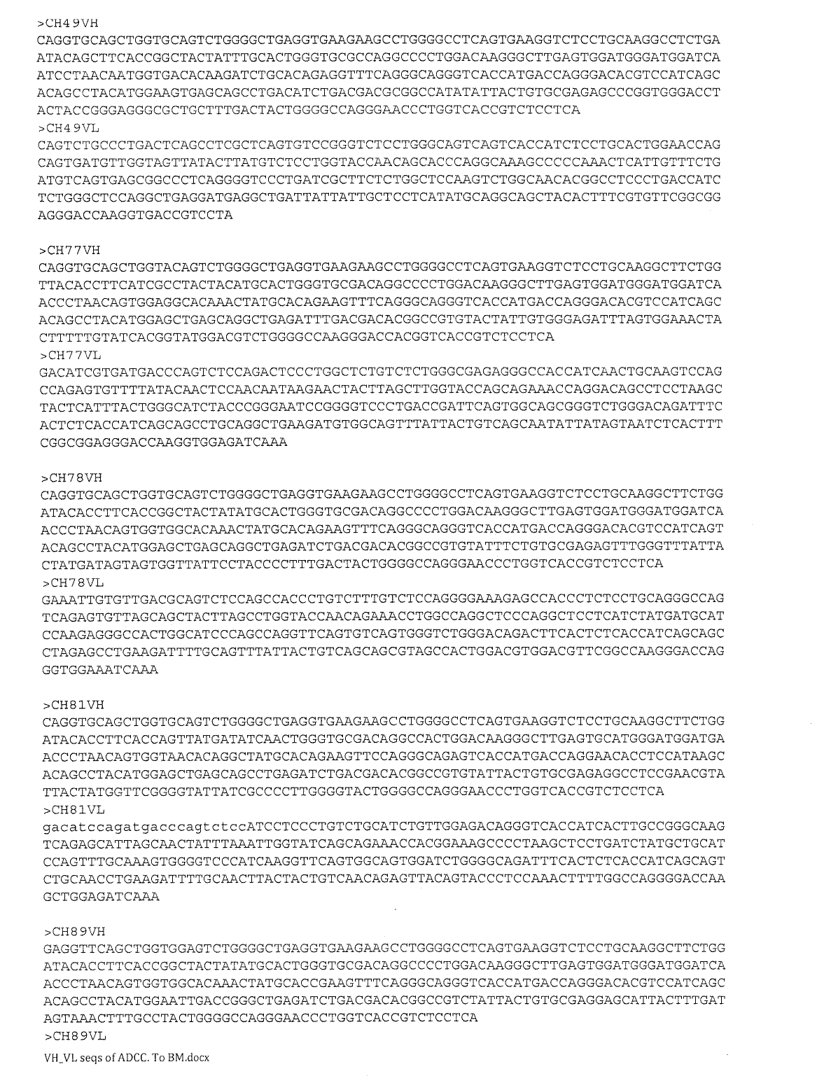

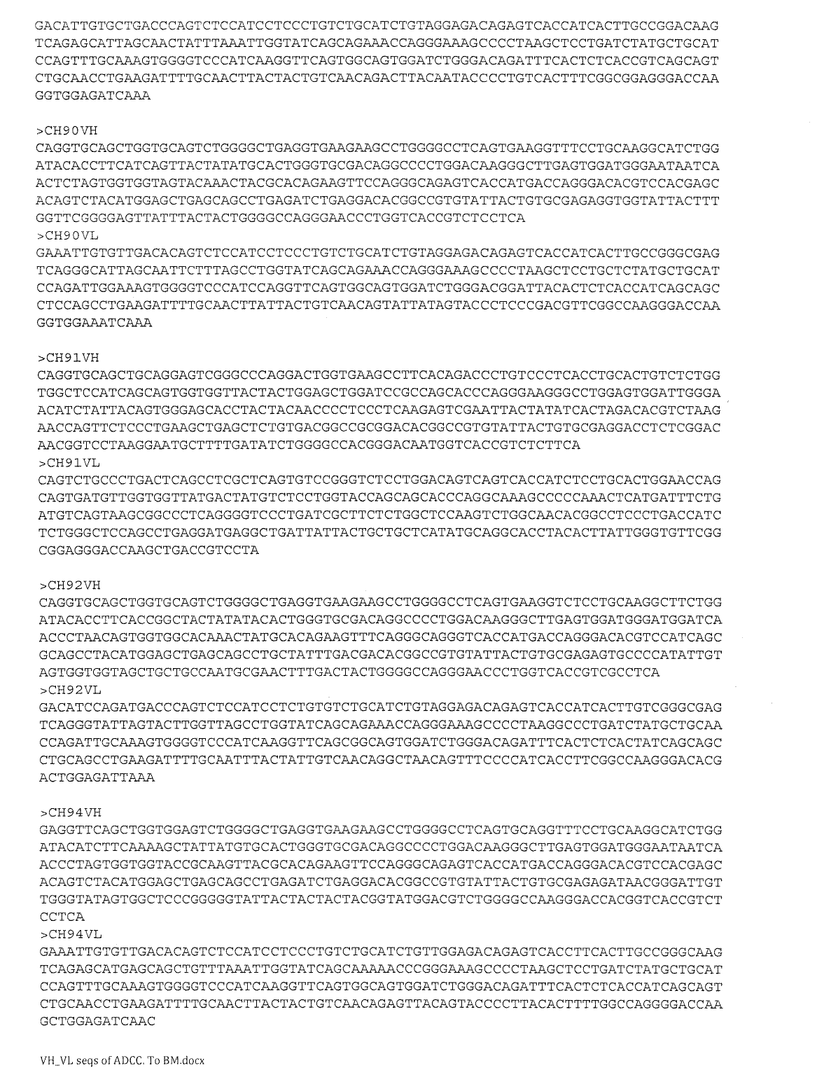

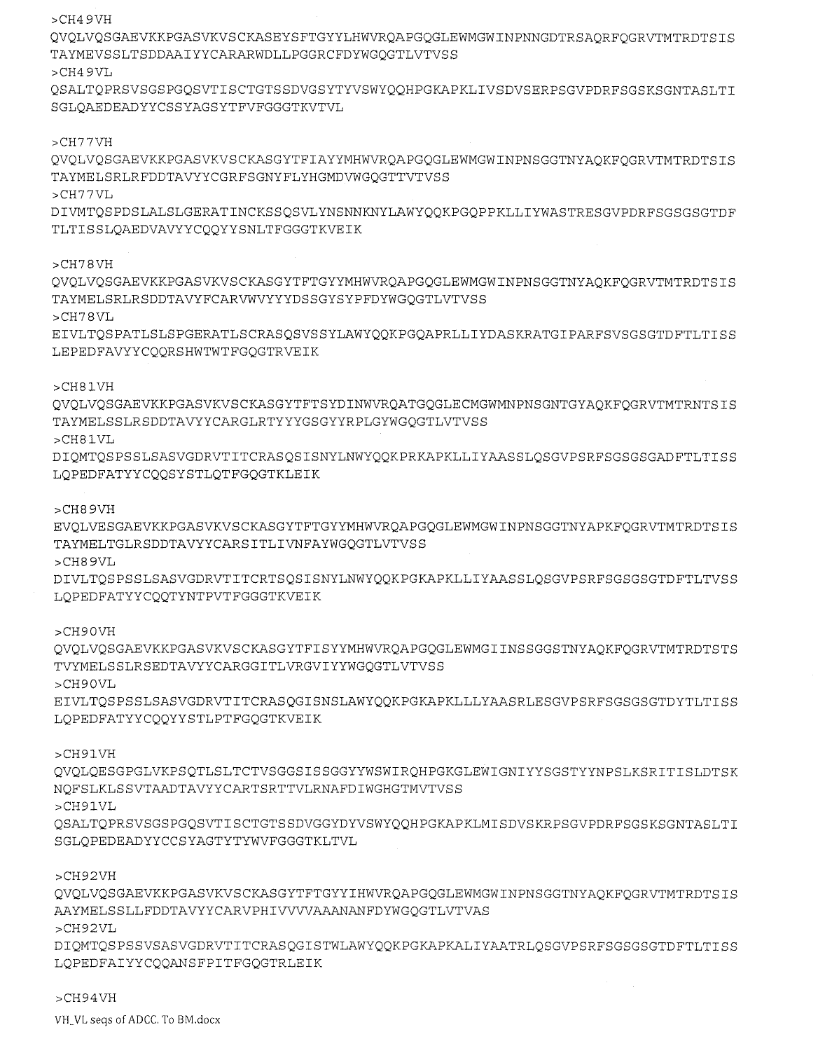

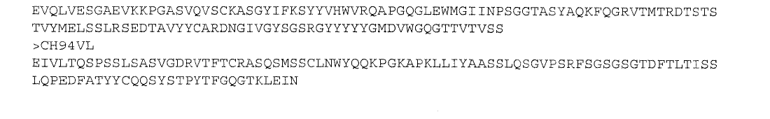

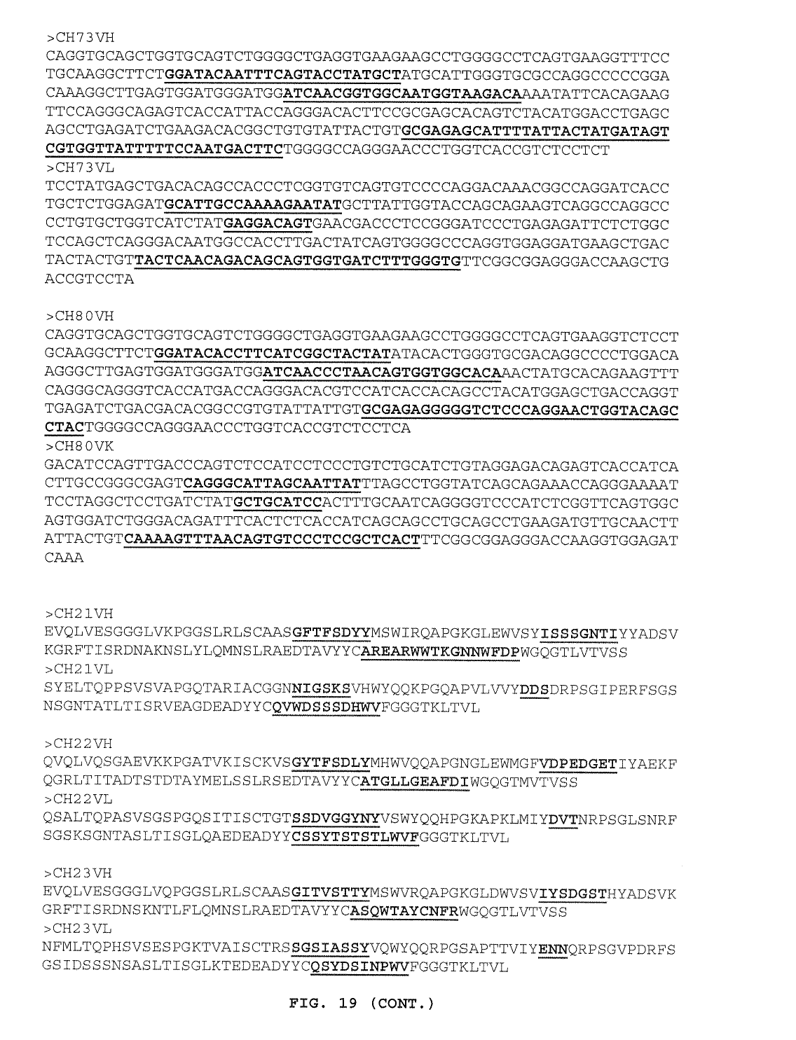

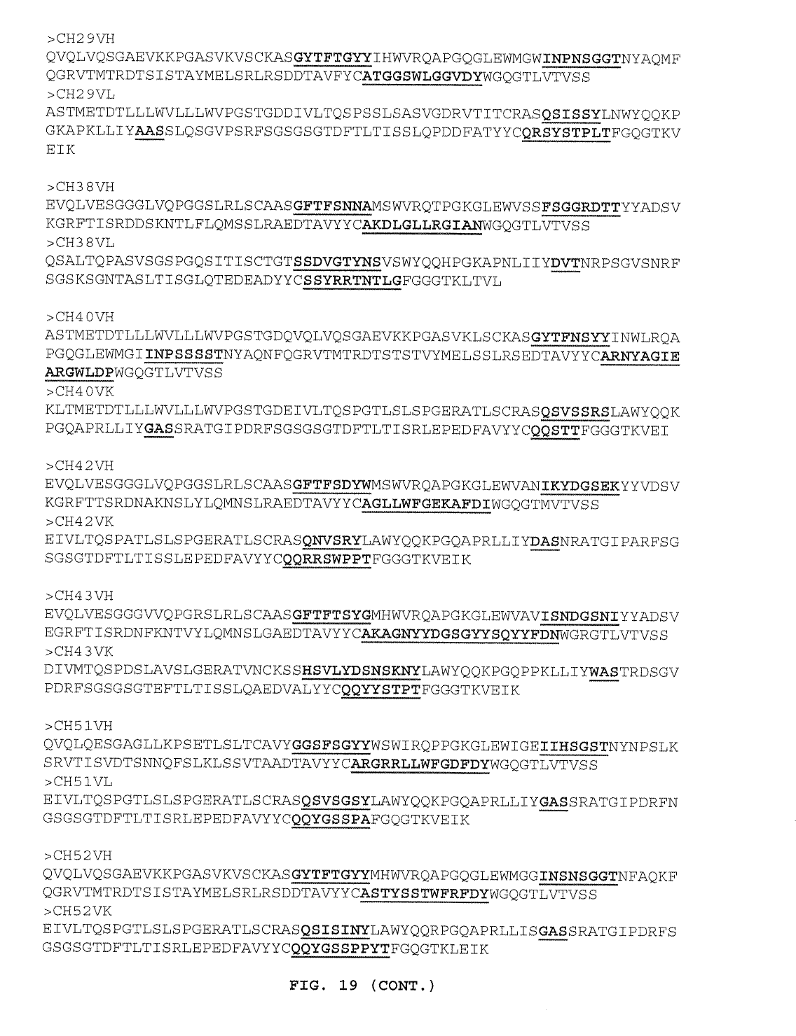

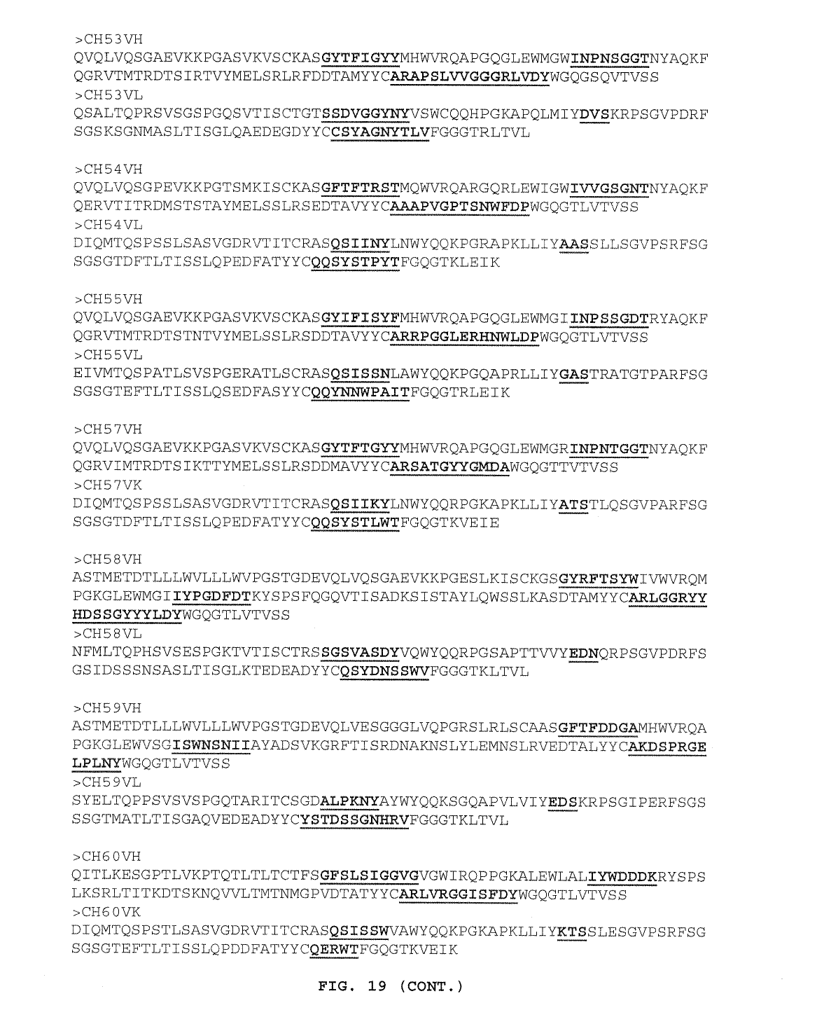

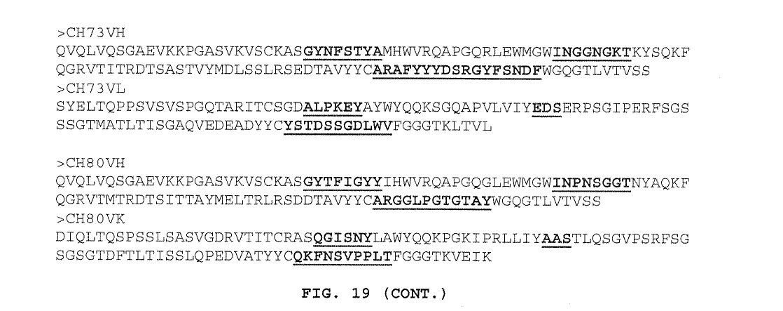

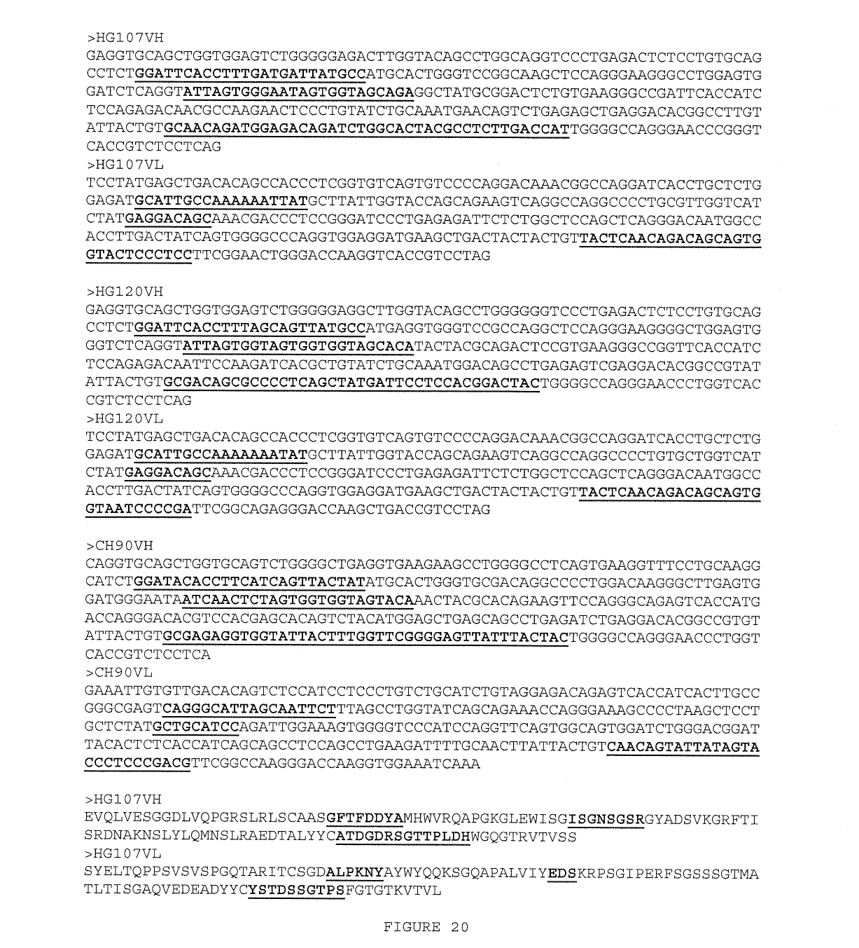

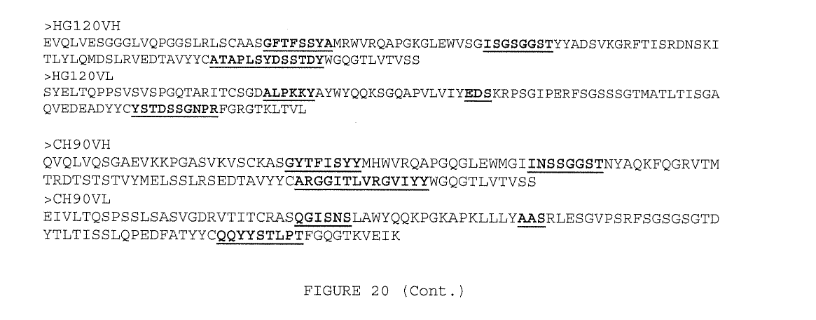

Antibodies for use in the invention include those comprising variable heavy (VH) and light (VL) chain amino acid sequences, for example but not limited to the sequences shown in FIGS. 9, 12, 19 and 20 (or comprising variable heavy and light chain amino acid sequences encoded by nucleic acid sequences shown in FIGS. 9-12, 19 and 20). In accordance with the methods of the present invention, either the intact antibody or a fragment thereof can be used. Either single chain Fv, bispecific antibody for T cell engagemen, or chimeric antigen receptors can be used (Chow et al, Adv. Exp. Biol. Med. 746:121-41 (2012)). That is, for example, intact antibody, a Fab fragment, a diabody, or a bispecific whole antibody can be used to inhibit HIV-1 infection in a subject (e.g., a human). A bispecific F(ab)₂ can also be used with one arm a targeting molecule like CD3 to deliver it to T cells and the other arm the arm of the native antibody (Chow et al, Adv. Exp. Biol. Med. 746:121-41 (2012)). Toxins that can be bound to the antibodies or antibody fragments described herein include unbound antibody, radioisotopes, biological toxins, boronated dendrimers, and immunoliposomes (Chow et al, Adv. Exp. Biol. Med. 746:121-41 (2012)). Toxins (e.g., radionucleotides or other radioactive species) can be conjugated to the antibody or antibody fragment using methods well known in the art (Chow et al, Adv. Exp. Biol. Med. 746:121-41 (2012)). The invention also includes variants of the antibodies (and fragments) disclosed herein, including variants that retain the ability to bind to recombinant Env protein, the ability to bind to the surface of virus-infected cells and/or ADCC-mediating properties of the antibodies specifically disclosed, and methods of using same to, for example, reduce HIV-1 infection risk. Combinations of the antibodies, or fragments thereof, disclosed herein can also be used in the methods of the invention. One combination of antibodies for the purpose of binding to virus-infected cells comprises A32+CH20+CH57 (see FIG. 10), another comprises 7B2 (see FIG. 11) together with at least one other antibody (or fragment) disclosed herein.

The antibodies, and fragments thereof, described above can be formulated as a composition (e.g., a pharmaceutical composition). Suitable compositions can comprise the ADCC-mediating antibody (or antibody fragment) dissolved or dispersed in a pharmaceutically acceptable carrier (e.g., an aqueous medium). The compositions can be sterile and can be in an injectable form (e.g., a form suitable for intravenous injection). The antibodies (and fragments thereof) can also be formulated as a composition appropriate for topical administration to the skin or mucosa. Such compositions can take the form of liquids, ointments, creams, gels and pastes. The antibodies (and fragments thereof) can also be formulated as a composition appropriate for intranasal administration. The antibodies (and fragments thereof) can be formulated so as to be administered as a post-coital douche or with a condom. Standard formulation techniques can be used in preparing suitable compositions.

The antibody (and fragments thereof), for example the ADCC-mediating antibodies, described herein have utility, for example, in settings including but not limited to the following:
i) in the setting of anticipated known exposure to HIV-1 infection, the antibodies described herein (or fragments thereof) and be administered prophylactically (e.g., IV, topically or intranasally) as a microbiocide,
ii) in the setting of known or suspected exposure, such as occurs in the setting of rape victims, or commercial sex workers, or in any homosexual or heterosexual transmission without condom protection, the antibodies described herein (or fragments thereof) can be administered as post-exposure prophylaxis, e.g., IV or topically, and
iii) in the setting of Acute HIV infection (AHI), the antibodies described herein (or fragments thereof) can be administered as a treatment for AHI to control the initial viral load or for the elimination of virus-infected CD4 T cells.

In accordance with the invention, the ADCC-mediating antibody (or antibody fragments) described herein can be administered prior to contact of the subject or the subject's immune system/cells with HIV-1 or within about 48 hours of such contact. Administration within this time frame can maximize inhibition of infection of vulnerable cells of the subject with HIV-1.

In addition, various forms of the antibodies described herein can be administered to chronically or acutely infected HIV patients and used to kill remaining virus infected cells by virtue of these antibodies binding to the surface of virus infected cells and being able to deliver a toxin to these reservoir cells. The A32 epitope is expressed early on in the life cycle of virus infection or reexpression (Ferrari, J. Virol. 85:7029-36 (2011); DeVico et al, J. Virol. 75:11096-105 (2001)).

Suitable dose ranges can depend on the antibody (or fragment) and on the nature of the formulation and route of administration. Optimum doses can be determined by one skilled in the art without undue experimentation. For example, doses of antibodies in the range of 1-50 mg/kg of unlabeled or labeled antibody (with toxins or radioactive moieties) can be used. If antibody fragments, with or without toxins are used or antibodies are used that can be targeted to specific CD4 infected T cells, then less antibody can be used (e.g., from 5 mg/kg to 0.01 mg/kg).

Antibodies of the invention and fragments thereof can be produced recombinantly using nucleic acids comprising nucleotide sequences encoding VH and VL sequences selected from those shown in FIGS. 9-12, 19 and 20.

Certain aspects of the invention can be described in greater detail in the non-limiting Examples that follow. (See also Provisional Applns. 61/613,222, filed Mar. 20, 2012 and 61/705,922 filed Sep. 26, 2012.)

### Example 1

### Experimental Details

### Plasma and Cellular Samples from Vaccine Recipients

All trial participants gave written informed consent as described for both studies (Nitayaphan et al, J. Infect. Dis. 190:702-706 (2004), Rerks-Ngarm et al, N. Engl. J. Med. 361:2209-2220 (2009)). Samples were collected and tested according to protocols approved by Institutional Review Boards at each site involved in these studies. Plasma samples were obtained from volunteers enrolled in the Phase I/II clinical trial (Nitayaphan et al, J. Infect. Dis. 190:702-706 (2004)) and in the community-based, randomized, multicenter, double-blind, placebo-controlled phase III efficacy trial (Rerks-Ngarm et al, N. Engl. J. Med. 361:2209-2220 (2009)); both trials tested the prime-boost combination of vaccines containing ALVAC-HIV (vCP1521) (Sanofi Pasteur) and AIDSVAX B/E (Global Solutions for Infectious Diseases). Plasma samples collected at enrollment (week 0) and two weeks after the last immunization (week 26) were selected by simple random sampling with a vaccine:placebo ratio of 40:10 for both men and women.

Peripheral blood mononuclear cells (PBMCs) from six vaccine recipients enrolled in the phase II (n=3) and phase III (n=3) trials whose plasma showed ADCC activity were used for isolation of memory B cells and monoclonal antibodies (mAbs). Subjects T141485, T141449 and T143859 participated in the phase II trial; subjects 609107, 210884 and 347759 were enrolled in the phase Ill trial. All six subjects had negative serology for HIV-1 infection at the time of collection.

Competition Binding Assay.

To determine the presence of A32 binding Ab in the plasma of the vaccine recipients, the previously described Full Length Single Chain (FLSC) assay was modified (DeVico et al, Proc. Natl. Acad. Sci. USA 104:17477-17482 (2007)). Briefly, biotinylated A32 was used at a limiting dilution of 0.173 μg/ml to compete the binding of plasma Ab to single chain complex (FLSC) captured (Aby D7324) on plate. Plasma from 80 vaccine recipients and 20 placebo recipients were initially screened at 1:50 final dilution. For plasma samples that blocked binding of biotinylated A32 mAb, the ability to mediate ≧50% of A32-blocking at 1:50 dilution was used as criterion for inclusion in this study. Seventy nine plasma samples met this criterion (data not shown) and were tested in a serial dilution to calculate the ID₅₀ titer.

ADCC-Luciferase (ADCC-92TH023) Assay.

Plasma was evaluated for ADCC activity against cells infected by HIV-1 92TH023 in an assay that employs a natural killer (NK) cell line as effectors. The NK cell line was derived from KHYG-1 cells (Japan Health Sciences Foundation) (Yagita et al, Leukemia 14:922-930 (2000)). These cells were transduced with a retroviral vector to stably express the V158 variant of human CD16a (FCGR3A). The target cells were CEM.NKR_{CCR5} cells (AIDS Research and Reference Reagent Program, Division of AIDS, NIAID, NIH, contributed by Dr. Alexandra Trkola) (Trkola et al, J. Virol. 73:8966-8974 (1999)), which were modified to express Firefly Luciferase upon infection. Target cells were infected with HIV-1 92TH023 by spinoculation (O'Doherty et al, J. Virol. 74:10074-10080 (2000)) 4 days prior to use in assays. NK effectors and 92TH023-infected targets were incubated at a 10:1 E:T ratio in the presence of triplicate serial dilutions of plasma for 8 hours. Wells containing NK cells and uninfected targets without plasma defined 0% relative light units (RLU), and wells with NK cells plus infected targets without plasma defined 100% RLU. ADCC activity was measured as the percentage loss of luciferase activity with NK cells plus infected targets in presence of plasma.

Recombinant gp120 HIV-1 Proteins.

Where indicated, CEM.NKR_{CCR5} target cells were coated with recombinant gp120 HIV-1 protein from the CM243 isolate representing the subtype A/E HIV-1 envelope (GenBank No. AY214109; Protein Sciences, Meiden, Conn.). The optimum amount to coat target cells was determined as previously described (Pollara et al, Cytometry A 79:603-612 (2011)).

Virus, Infectious Molecular Clones (IMC) for ADCC GTL Assay.

HIV-1 reporter viruses used were replication-competent IMCs designed to encode subtypes A/E, B or C env genes in cis within an isogenic backbone that also expresses the Renilla luciferase reporter gene and preserves all viral open reading frames (Edmonds et al, Virology 408:1-13 (2010)). The Env-IMC-LucR viruses used were: subtype A/E NL-LucR.T2A-AE.CM235-ecto (IMC_{CM235}) (GenBank No. AF2699954; plasmid provided by Dr. Jerome Kim, US Military HIV Research Program), subtype B NL-LucR.T2A-BaL.ecto (IMC_{BaL}) (Adachi et al, J. Virol. 59:284-291 (1986)), subtype C NL-LucR.T2A-DU422.ecto (IMC_{DU422}; GeneBank No. DQ411854), and subtype C NL-LucR.T2A-DU151.ecto (IMC_{DU151}; GeneBank No. DQ411851). Reporter virus stocks were generated by transfection of 293T cells with proviral IMC plasmid DNA and titrated on TZM-bl cells for quality control.

ADCC-GTL Assay.

Antibody Dependent Cellular Cytotoxic (ADCC) activity was detected according to the previously described ADCC-GranToxiLux (GTL) procedure (Pollara et al, Cytometry A 79:603-612 (2011)). The following target cells were used: CM243 gp120-coated (ADCC-CM243 assay), IMC_{CM235}-, IMC_{BaL}-, IMC_{CU422}-, and IMC_{DU151}- infected CEM.NKR_{CCR5} (ADCC-E.CM235, ADCC-B.BaL, ADCC-C.DU422, and ADCC-C.DU151 assay, respectively) (Trkola et al, J. Virol. 73:8966-8974 (1999)). All the PBMC samples from the seronegative donors used as effector cells were obtained according to the appropriate Institutional Review Board protocol. Ten thousand target cells per well were used and effector to target (E:T) ratios of 30:1 and 10:1 were used for whole PBMC and purified NK effector cells, respectively. MAb A32 (James Robinson; Tulane University, New Orleans, La.), Palivizumab (MedImmune, LLC; Gaithersburg, Md.; used as negative control) and vaccine induced mAbs were tested as six 4-fold serial dilutions starting at a concentration of 40 μg/ml (range 40-0.039 μg/ml). For the Fab blocking assay, the target cells were incubated for 15 min at room temperature in the presence of 10 g/ml A32, 19B (Moore et al, Bioinformatics 26:867-872 (1995)), and 17B (Thali et al, J. Virol. 67:3978-3988 (1993)) Fab fragments, produced by Barton Haynes. The excess Fab were removed by washing the target cell suspensions once before plating with the effector cells as previously described (Ferrari et al, J. Virol. 85:7029-7036 (2011)). A minimum of 2.5×10³ events representing viable gp120-coated or infected target cells was acquired for each well. Data analysis was performed using FlowJo 9.3.2 software. The results are expressed as % GzB activity, defined as the percentage of cells positive for proteolytically active GzB out of the total viable target cell population. The final results are expressed after subtracting the background represented by the % GzB activity observed in wells containing effector and target cell populations in absence of mAb, IgG preparation, or plasma. The results were considered positive if %/GzB activity after background subtraction was >8% for the gp120-coated or was >5% for the CM235-infected target cells.

Isolation of ADCC-Mediating Monoclonal Antibodies.

Monoclonal antibodies were isolated either from IgG⁺ memory B cells cultured at near clonal dilution for 14 days (Bonsignori et al, J. Virol. 85:9998-10009 (2011)) followed by sequential screenings of culture supernatants for HIV-1 gp120 Env binding and ADCC activity or from memory B cells that bound to HIV-1 group M consensus gp140_{Con.S} Env sorted by flow cytometry (Gray et al, J. Virol. 85:7719-7729 (2011)).

Subject 210884 was tested using IgG memory B cell cultures isolated and cultured at near clonal dilution as previously described (Bonsignori et al, J. Virol. 85:9998-10009 (2011)). Briefly, 57,600 IgG⁺ memory B cells were isolated from frozen PBMCs by selecting CD2(neg), CD14(neg), CD16(neg), CD235a(neg), IgD(neg) and IgG(pos) cells through two rounds of separation with magnetic beads (Miltenyi Biotec, Auburn, Calif.) and resuspended in complete medium containing 2.5 μg/ml oCpG ODN2006 (tlrl-2006, InvivoGen, San Diego, Calif.), 5 μM CHK2 kinase inhibitor (Calbiochem/EMD Chemicals, Gibbstown, N.J.) and EBV (200 μl supernatant of B95-8 cells/10⁴ memory B cells). After overnight incubation in bulk, cells were distributed into 96-well round-bottom tissue culture plates at a cell density of 8 cells/well in presence of ODN2006, CHK2 kinase inhibitor and irradiated (7500 cGy) CD40 ligand-expressing L cells (5000 cells/well). Cells were re-fed at day 7 and harvested at day 14.

Subjects T141485, T141449, T143859 and 609107 were tested using antigen-specific memory B cell sorting as previously described (Gray et al, J. Virol. 85:7719-7729 (2011)), with the following modifications. Group M consensus gp140_{Con.S} Env labeled with Pacific Blue and Alexa Fluor 647 (Invitrogen, Carlsbad, Calif.) was used for sorting. Memory B cells were gated as Aqua Vital Dye(neg), CD3(neg), CD14(neg), CD16(neg), CD235a(neg), CD19(pos), and surface IgD(neg); memory B cells stained with gp140_{Con.S} in both colors were sorted as single cells as described (Gray et al, J. Virol. 85:7719-7729 (2011)). A total of 137,345 memory B cells were screened using this method: 32,766 from subject T141485; 54,621 from subject T141449; 20,629 from subject T143859 and 29,329 from subject 609107.

For subject 347759, memory B cells were screened using both methods: 57,600 cells were cultured at near clonal dilution and 69,400 memory B cells were sorted. Sorted cells were previously enriched for IgG⁺ memory B cells as described above, incubated overnight in complete medium containing 2.5 μg/ml oCpG ODN2006, 5 μM CHK2 kinase inhibitor and EBV (200 μl supernatant of B95-8 cells/10⁴ memory B cells) and then stimulated for 7 days at a cell density of 1,000 cells/well in presence of ODN2006, CHK2 kinase inhibitor and irradiated CD40 ligand-expressing L cells (5,000 cells/well).

Isolation of V(D)J Immunoglobulin Regions.

Single cell PCR was performed as previously described (Liao et al, J. Virol. Methods 158:171-179 (2009), Wrammert et al, Nature 453:667-671 (2008)). Briefly, reverse transcription (RT) was performed using Superscript III reverse transcriptase (Invitrogen, Carlsbad, Calif.) and human constant region primers for IgG, IgA₁, IgA₁, IgA₂, IgM, IgD, Igκ, Igλ; separate reactions amplified individual V_{H}, V_{κ}, and V_{λ} families from the cDNA template using two rounds of PCR. Products were analyzed with agarose gels (1.2%) and purified with PCR purification kits (QIAGEN, Valencia, Calif.). Products were sequenced in forward and reverse directions using a BigDye® sequencing kit using an ABI 3700 (Applied Biosystems, Foster City, Calif.). Sequence base calling was performed using Phred (Ewing and Green, Genome Res. 8:186-194 (1998), Ewing et al, Genome Res. 8:175-185 (1998)); forward and reverse strands were assembled using an assembly algorithm based on the quality scores at each position (Munshaw and Kepler, Bioinformatics 26:867-872 (2010)). The estimated PCR artifact rate was 0.28 or approximately one PCR artifact per five genes amplified. Ig isotype was determined by local alignment with genes of known isotype (Smith and Waterman, J. Mol. Biol. 147:195-197 (1981)); V, D, and J region genes, CDR3 loop lengths, and mutation rates were identified using SoDA (Volpe et al, Bioinformatics 22:438-444 (2006)) and data were annotated so that matching subject data and sort information was linked to the cDNA sequence and analysis results.

Expression of Recombinant Antibodies.

Isolated Ig V(D)J gene pairs were assembled by PCR into linear full-length Ig heavy- and light-chain gene expression cassettes (Liao et al, J. Virol. Methods 158:171-179 (2009)) and optimized as previously described for binding to the Fcγ-Receptors (Shields et al, J. Biol. Chem. 276:6591-6604 (2001)). Human embryonic kidney cell line 293T (ATCC, Manassas, Va.) was grown to near confluence in 6-well tissue culture plates (Becton Dickson, Franklin Lakes, N.J.) and transfected with 2 μg per well of purified PCR-produced IgH and IgL linear Ig gene expression cassettes using Effectene (Qiagen). The supernatants were harvested from the transfected 293T cells after three days of incubation at 37° C. in 5% CO₂ and the monoclonal antibodies were purified as previously described (Liao et al, J. Virol. Methods 158:171-179 (2009)).

Direct Binding ELISAs.

Three-hundred eighty four-well plates (Corning Life Sciences, Lowell, Mass.) were coated overnight at 4° C. with 15 μl of purified HIV-1 monomeric gp120 envelope glycoproteins (E.A244 gp120, B.MN gp120 and A.92TH023 gp120) antigen at 2 μg/ml and blocked with assay diluent (PBS containing 4% (w/v) whey protein/i 5% normal goat serum/0.5% Tween 20/0.05% sodium azide) for 1 hour at room temperature.

Ten μl/well of purified mAbs were incubated for 2 hours at room temperature either in serial 3-fold dilutions starting at 100 g/ml for the determination of EC₅₀ concentrations and then washed with PBS/0.1% Tween 20. Thirty μl/well of alkaline phosphatase-conjugated goat anti-human IgG in assay diluent was added for 1 hour, washed and detected with 30 μl/well of p-Nitrophenyl Phosphate Substrate diluted in 50 mM NaHCO₃+Na₂CO₃ (1:1 v/v) pH 9.6/10 mM MgCl₂. Plates were developed for 45 minutes in the dark at room temperature and read at OD₄₀₅ with a VersaMax microplate reader (Molecular Devices, Sunnyvale, Calif.).

Epitope mapping studies were performed using 15-mer linear peptides spanning the gp120 envelope glycoprotein of the MN and 92TH023 HIV-1 strains obtained from the AIDS Reagent Repository as coating antigens, horseradish peroxidase goat anti-human IgG as secondary antibody and 3,3′,5,5′-Tetramethylbenzidine [TMB] Substrate for detection.

Statistical analyses. The analysis of the ADCC-mediating Ab responses in the plasma of the vaccine recipients was conducted as following. For each time point of a subject, partial area under the activity versus log 10(dilution) curve [AUC] is estimated nonparametrically for each assay. For ADCC-CM243 assay using gp120-coated target cell, AUC is calculated based on % GzB activity across dilution levels 50, 250, 1250, 6250, 31250, and 156250; for ADCC-92TH023 assay using infected cells, AUC is calculated based on % loss of Luciferase activity across dilution levels 32, 100, 316, 1000. Two-sample t-test allowing for unequal variance is used to test the mean difference in AUC between the vaccine and placebo groups at Week 26. Paired t-test is used to test the mean difference in AUC between Week 26 time-point and Week 0 time-point among vaccines. For each of the vaccine and placebo groups and for each time-point, the positive response rate is estimated by the observed fraction of subjects that have a positive response (defined as peak %/GzB greater than 8% for ADCC-CM243 assay and peak % loss of Luciferase activity greater than 9% for ADCC-92TH023 assay). A 95% confidence interval (computed by the Agresti-Coull method) is provided around each response rate. An exact p-value from McNemar's test is used to evaluate whether the response rate differs for the Week 26 time-point versus the Week 0 time-point among vaccines. Fisher's exact test is used to provide a p-value to test whether the response rate differs between the vaccine and placebo groups at Week 26.

The other statistical analyses conducted in this study were performed using the Prism software v5.0c (GraphPad Software, Inc) and the appropriate methods are listed throughout the manuscript

Results

Vaccine-Induced ADCC Responses.

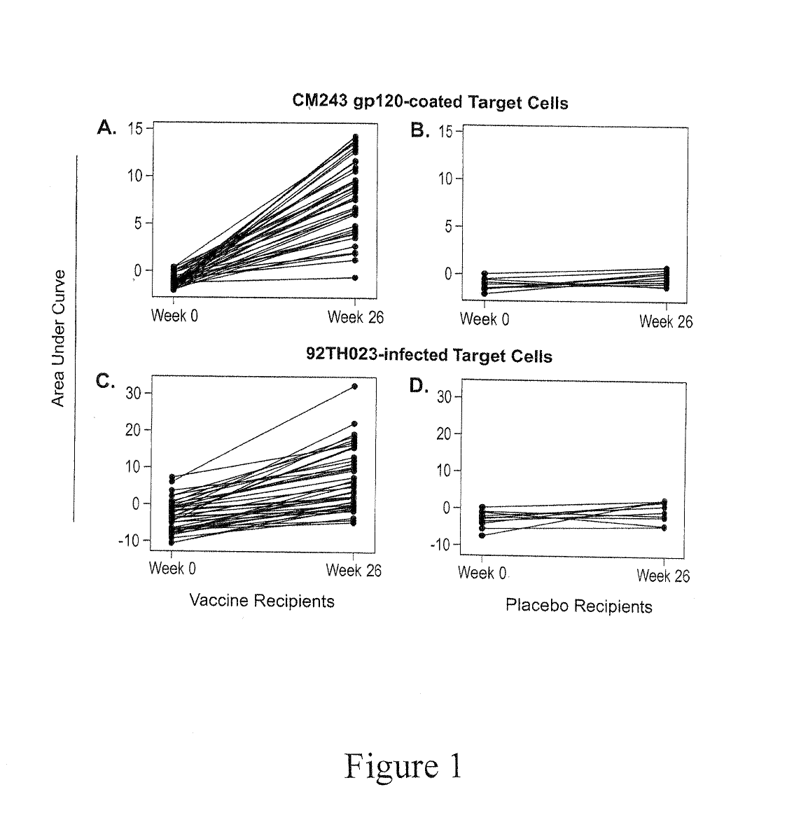

A study was made of 50 simple random sampled plasma specimens drawn from subjects enrolled in the RV144 vaccine trial at enrollment (week 0) and two weeks after the last immunization (week 26): 10 placebo recipients (5 male and 5 female) and 40 vaccine recipients (20 male and 20 female; four injections of a recombinant canarypox vector vaccine (ALVAC-HIV [vCP1521]) and two booster injections of recombinant gp120 subunit (AIDSVAX B/E)) (Nitayaphan et al, J. Infect. Dis. 190:702-706 (2004), Rerks-Ngarm et al, N. Engl. J. Med. 361:2209-2220 (2009)). The frequency of ADCC responders (Table 1) and the area under the curve [AUC] for ADCC activity (FIG. 1A-D) of both vaccine and placebo recipients were measured using two ADCC assays: CEM.NKR_{CCR5} target cells either coated with HIV-1 AE.CM243 gp120 [ADCC-CM243](Pollara et al, Cytometry A 79:603-612 (2011)) or infected with the AE.92TH023 HIV-1 strain [ADCC-92TH023](Haynes, Case-control study of the RV144 trial for immune correlates: the analysis and way forward, abstr., p. AIDS Vaccine Conference, Bangkok, Thailand, Sep. 12-15, 2011).

TABLE 1
Frequency of ADCC responders among vaccine and placebo
recipients before and after vaccination

| | | | ADCC-92TH023 |
|---|---|---|---|
| | | ADCC-CM243 assay | assay |
| | | N (%, 95% CI) | N (%, 95% CI) |
| Vaccine | Week 0 | 0 (0%, 0-31%) | 4 (10%, 2.8-23.7%) |
| Recipients | Week 26 | 36 (90%, 76-97%) | 29 (72.5%, 56.1-85.4%) |
| (n = 40) | | | |
| Placebo | Week 0 | 1 (10%, 0-44.5%) | 0 (0%, 0-31%) |
| Recipients | week 26 | 1 (10%, 0-44.5%) | 1 (10%, 0.3-44.5%) |
| (n = 10) | | | |

The ADCC response rate measured with the ADCC-CM243 assay increased from 0% at week 0 to 90% at week 26 among the vaccine recipients (Table 1). Similarly, the ADCC-92TH023 assay detected activity in 72.5% (29/40) of vaccine recipients at week 26 (Table 1). For both assays, the frequency of positive responses among the vaccine recipients was significantly higher comparing baseline (week 0) to post immunization (week 26) (p<0.0001 for both assays).

An evaluation was made of AUC of a dilution of antibody in the assay (see statistical analysis section above). In both the ADCC-CM243 and ADCC-92TH023 assays, AUC values of vaccinated subjects at week 26 were significantly higher than both those in the vaccine recipients at week 0 and in the placebo group at week 26 (p<0.0001 and p<0.001, respectively) (FIGS. 1A-1D). Thus, the ALVAC-HIV/AIDSVAX B/E vaccine induced anti-HIV-1 gp120 ADCC activity in ˜70-90% of vaccine recipients, depending on the assay utilized. This frequency of responders among vaccines is similar to that reported in earlier Phase 11 studies as well as in RV144 (Haynes, Case-control study of the RV144 trial for immune correlates: the analysis and way forward, abstr., p. AIDS Vaccine Conference, Bangkok, Thailand, Sep. 12-15, 2011, Karnasuta et al, Vaccine 23:2522-2529 (2005)). It is important to note that the 92TH023-infected target cell ADCC assay was used in the RV144 immune correlates primary analysis and, in the secondary analysis, high activity in this assay associated with low plasma anti-Env IgA responses inversely correlated with infection risk (Haynes, Case-control study of the RV144 trial for immune correlates: the analysis and way forward, abstr., p. AIDS Vaccine Conference, Bangkok, Thailand, Sep. 12-15, 2011).

Plasma ADCC Activity is Blocked in Part by mAb A32.

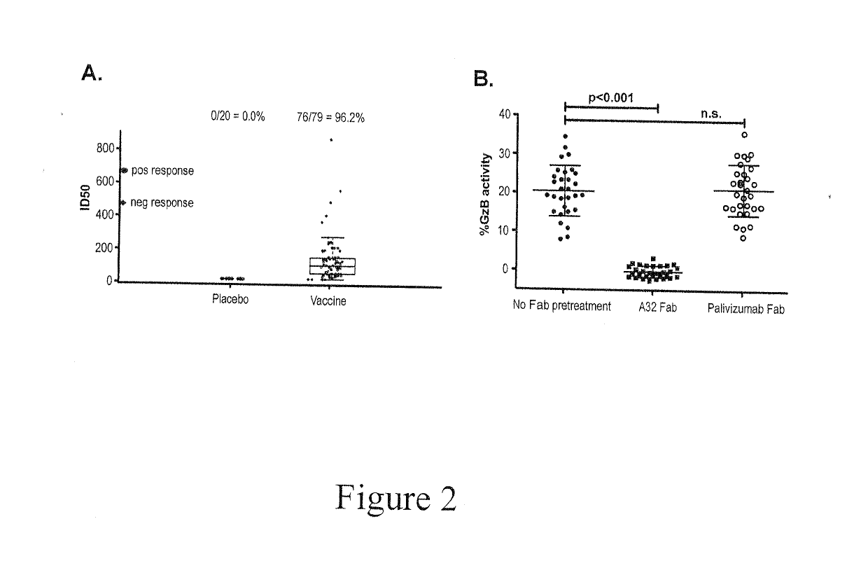

Since mAb A32 can block plasma ADCC responses during chronic infection (Ferrari et al, J. Virol. 85:7029-7036 (2011)), a determination was made as to whether A32-like antibodies were produced by RV144 vaccine recipients. An evaluation was first made of the ability of plasma samples collected at week 26 post-vaccination from simple random samples drawn from both RV144 vaccine (n=79 out of 80; one sample was not studied because of less than 50% inhibition at screening) and placebo (n=20) recipients for their ability to block the binding of biotinylated-A32 mAb to B.BaL Env. Plasma Ab blocked A32 mAb binding in 76/79 (96.2%) of the vaccine recipients with an average 50% inhibitory dose [ID₅₀] titer of 119 (95% CI=95-130) (FIG. 2A). These data demonstrated the presence of A32-like antibodies in the plasma of vaccine recipients.

An evaluation was then made of the effect of pre-treatment of CM243 gp120-coated target cells with A32 Fab on plasma-mediated ADCC (Ferrari et al, J. Virol. 85:7029-7036 (2011)). Thirty vaccine recipients whose plasma was previously identified to mediate ADCC were selected to represent each tertile (low, medium and high response) of the range of ADCC activities observed. These plasma samples were tested to determine the dilution that provided maximum ADCC activity (data not shown). When tested at the optimal dilution, these plasmas induced granzyme B [GzB] activity against AE.CM243 gp120-coated target cells ranging from 8.0% to 34.6% (mean±SD=20.4 f 6.6; FIG. 2B). When the cells were pre-treated with 10 μg/mL of A32 Fab, ADCC activity was reduced or completely abrogated for each plasma sample (GzB activity ≦3.2%, p<0.001 vs. untreated; FIG. 2B). Similar treatment with a control Fab made from Palivizumab (Johnson et al, J. Infect. Dis. 176:1215-1224 (1997)) did not affect plasma ADCC activity (range 9.0-35.8%; mean+SD=21.1±6.7%; FIG. 2B). However, pre-incubation with 10 μg/mL and 50 μg/mL of A32 Fab did not block plasma ADCC activity at peak of responses (1:50 dilution) in ADCC assays using target cells infected with either the E.92TH023 or the E.CM235 HIV-1 strains (data not shown): This lack of inhibition may be due to unfavorable kinetics for Fab epitope recognition on infected cells in the presence of polyclonal antibodies in plasma. To better define the nature of the antibodies responsible for the observed ADCC activity, ADCC-mediating mAbs were isolated from ALVAC-HIV/AIDSVAX B/E vaccine recipients.

Isolation of ADCC-Mediating Antibodies from ALVAC-HIV/AIDSVAX B/E Vaccines.

A total of 23 mAbs that mediated ADCC were isolated from memory B cells of six vaccine recipients enrolled in the RV135 phase II (n=3) (Karnasuta et al, Vaccine 23:2522-2529 (2005), Nitayaphan et al, J. Infect. Dis. 190:702-706 (2004)) or RV144 phase III (n=3) (Rerks-Ngarm et al, N. Engl. J. Med. 361:2209-2220 (2009)) ALVAC-HIV/AIDSVAX B/E clinical trials. Nine mAbs (CH49, CH51, CH52, CH53, CH54, CH55, CH57, CH58 and CH59) were obtained from cultured IgG⁺ memory B cells that bound to one or more of the E.A244, B.MN and E.92TH023 gp120 envelope glycoproteins, while the remaining 14 were obtained from group M consensus gp140_{CON-S} Env-specific flow cytometric single memory B cell sorting (Bonsignori et al, J. Virol. 85:9998-10009 (2011), Gray et al, J. Virol. 85:7719-7729 (2011)). Two of the 23 ADCC-mediating mAbs were against the gp120 Env V2 region and are the subject of a separate report (Liao, Bonsignori, Haynes et al., submitted).

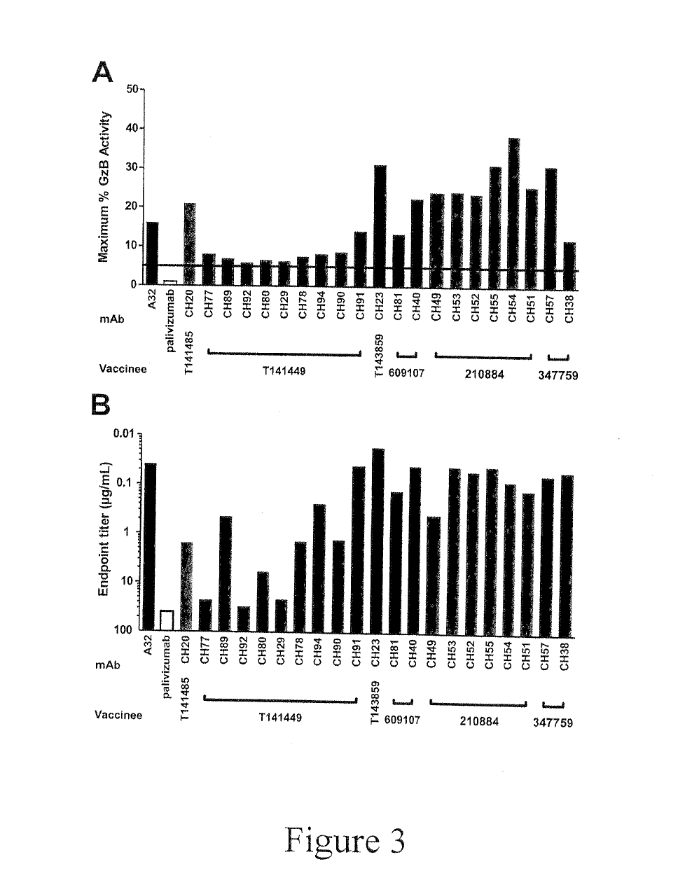

ADCC activity of the remaining 21 mAbs, purified and expressed in a codon-optimized IgG₁ backbone, was measured using both E.CM243 gp120-coated [ADCC-CM243] and E.CM235-infected [ADCC-CM235] target cells in the flow-based assay described above. The maximum % GzB activity of the 21 mAbs ranged from 38.9% (CH54) to 6.0% (CH92) (FIG. 3A). Remarkably, 11/21 mAbs displayed a maximum % GzB activity greater than that of A32 mAb (16%) in duplicate assays: CH54 (38.9%), CH55 (31.4%), CH57 (31.3%), CH23 (31.2%), CH49 (26.7%), CH51 (25.9%), CH53 (24.4%), CH52 (23.9%), CH40 (22.6%), and CH20 (21.0%). The endpoint titers of each of the 21 mAbs (FIG. 3B) ranged from <20 ng/mL to 30.3 g/mL (mean±SD=4.1±8.8 μg/mL).

None of the ADCC-mediating mAbs were heavily somatically mutated: the mean nucleotide mutation frequencies of the heavy and light chains were 2.4% (range: 0.5-5.1%) and 1.8% (range: 0.4-4.3%), respectively (Table 2). These data demonstrate that the ALVAC-HIV/AIDSVAX B/E vaccine induced polyclonal antibody responses capable of mediating moderate to high levels of ADCC activity without requiring high levels of ADCC antibody affinity maturation.

TABLE 2
Characteristics of the V(D)J rearrangements of vaccine-induced ADCC-mediating monoclonal antibodies

| | | Heavy Chain | | | | | | Light Chain | | | | |
|---|---|---|---|---|---|---|---|---|---|---|---|---|
| PTID¹ | mAb ID | Isotype | V | D | J | CDR3² | Mutation³ | Type | V | J | CDR3² | Mutation³ |
| T141485 | CH20 | G1 | 1-69*02 | 6-6*01 | 4*02 | 15 | 2.6% | λ | 2-23*02 | 3*02 | 10 | 0.4% |
| T141449 | CH77 | G3 | 1-2*02 | 2-OF15*02 | 6*02 | 15 | 2.3% | κ | 4-1*01 | 4*01 | 8 | 0.8% |
| | CH89 | G3 | 1-2*02 | 3-22*01 | 4*02 | 12 | 2.1% | κ | 1-39-*01 | 4*01 | 9 | 1.4% |
| | CH92 | G1 | 1-2*02 | 2-15*01 | 4*02 | 19 | 1.7% | κ | 1D-12*01 | 5*01 | 9 | 2.6% |
| | CH80 | G1 | 1-2*02 | 1-IR1*01C | 4*02 | 12 | 1.6% | κ | 1-27*01 | 4*01 | 10 | 1.1% |
| | CH29 | A2 | 1-2*02 | 2-15*01 | 4*02 | 12 | 0.8% | κ | 1-39*01 | 1*01 | 9 | 0.6% |
| | CH78 | G1 | 1-2*02 | 3-22*01 | 4*02 | 19 | 0.7% | κ | 3-11*01 | 1*01 | 9 | 1.1% |
| | CH94 | G1 | 1-46*02 | 5-12*01 | 6*02 | 23 | 2.2% | κ | 1-39*01 | 2*01 | 9 | 1.7% |
| | CH90 | G1 | 1-46*01 | 3-10*01 | 4*02 | 14 | 1.5% | κ | 1-13*02 | 1*01 | 9 | 4.3% |
| | CH91 | G1 | 4-31*03 | 4-17*01 | 3*02 | 15 | 2.0% | λ | 2-11*01 | 3*02 | 11 | 1.4% |
| T143859 | CH23 | G1 | 3-66*01 | 3-OR15*3 | 1*01 | 11 | 4.5% | λ | 6-57*01 | 3*02 | 10 | 2.2% |
| 609107 | CH81 | G1 | 1-8*01 | 3-10*01 | 4*02 | 19 | 0.5% | κ | 1-39*01 | 2*01, 02 | 9 | 1.4% |
| | CH40 | G1 | 1-46*02 | 6-6*01 | 5*02 | 15 | 3.6% | κ | 3-20*01 | 4*01 | 5 | 0.9% |
| 210884 | CH49 | G1 | 1-2*02 | 1-26*01 | 4*02 | 16 | 5.1% | λ | 2-11*01 | 3*02 | 10 | 3.1% |
| | CH53 | G1 | 1-2*02 | 2-2*01, 02 | 4*02 | 16 | 2.3% | λ | 2-11*01 | 2*01 | 10 | 2.4% |
| | CH52 | G1 | 1-2*02 | 6-13*01 | 4*02 | 13 | 1.4% | κ | 3-20*01 | 2*01 | 10 | 1.8% |
| | CH55 | G1 | 1-46*01 | 1-1*01 | 5*02 | 15 | 4.3% | κ | 3-15*01 | 5*01 | 10 | 1.5% |
| | CH54 | G1 | 1-58*02 | 1-26*01 | 5*02 | 14 | 2.1% | κ | 1-39*01 | 2*01 | 9 | 1.4% |
| | CH51 | G1 | 4-34*12 | 3-10*01 | 4*02 | 14 | 0.5% | κ | 3-20*01 | 1*01 | 8 | 0.6% |
| 347759 | CH57 | G1 | 1-2*02 | 1-1*01 | 6*02 | 12 | 3.4% | κ | 1-39*01 | 1*01 | 9 | 4.0% |
| | CH38 | A1 | 3-23*01 | 3-10*01, 02 | 1*01 | 12 | 4.7% | λ | 2-14*03 | 3*02 | 10 | 3.6% |

¹PTID = Participant ID.
²CDR3 = Complementarity Determining Region 3, length is expressed as amino acids according to the Kabat numbering system (20).
³Nucleotide mutation frequency in V gene as determined by SoDA (48).

Epitope Mapping of Vaccine-Induced ADCC-Mediating Antibodies.

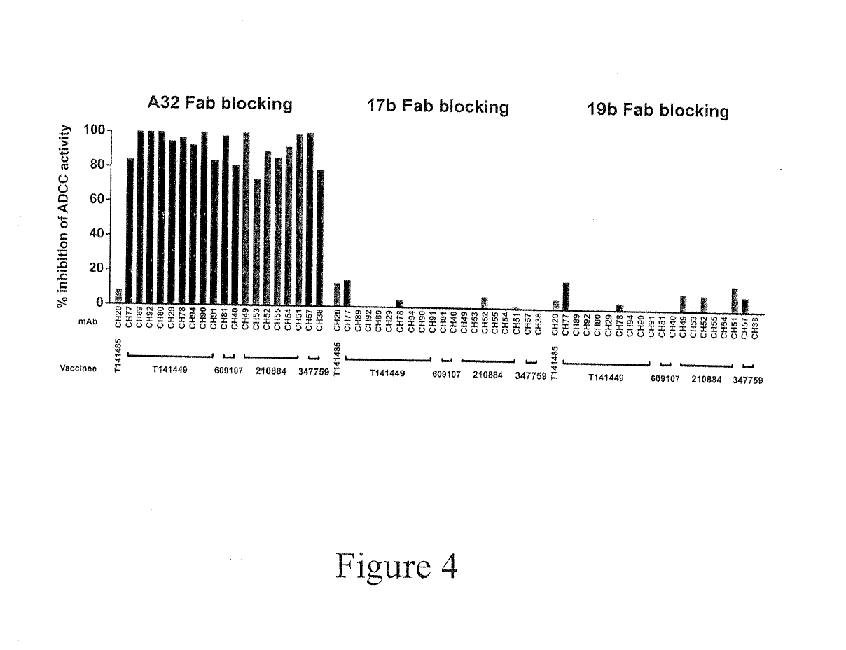

To define the specificity of ADCC-mediating mAbs, a determination was made as to whether they recognized linear epitopes by testing their ability to bind to overlapping linear peptides spanning the gp120 envelope glycoprotein of the B.MN or E.92TH023 HIV-1 strains. Each mAb bound to one or more of the vaccine gp120 envelope glycoproteins, which included the B.MN and E.92TH023 strains (Table 3). It was found that 19/20 mAbs (CH53 was not tested) did not react with any of the B.MN or E.92TH023 peptides, while one (CH23) reacted with the clade E V3 loop (NTRTSINIGRGQVFY). As previously described, the A32 Fab blocking strategy was used in the ADCC-CM235 assay to determine whether the ADCC activity of the 20 mAbs not specific for the V3 loop was mediated by targeting conformational epitopes expressed on infected cells that could be blocked by the A32 mAb (FIG. 4). As a control, the ability of these 20 mAbs to block the ADCC activity mediated by 17B and 19B Fab fragments, which target the CD4-induced [CD4i] and the V3 epitopes, respectively (FIG. 4), was tested. In contrast to plasma ADCC activity, which could not be blocked by A32 when tested against CM235-infected target cells, A32 Fab blocking inhibited between 73% and 100% (mean±SD=92%±9%) of the ADCC activity mediated by 19/20 (95%) non-V3 mAbs (FIG. 4). CH20 was not inhibited by any of A32, 17B, or 19B Fab fragments (FIG. 4). None of the mAbs displayed substantial loss of ADCC activity (defined as >20% inhibition) when E.CM235-infected target cells were pre-incubated with Fab fragments of mAb 17B or 19B (FIG. 4).

TABLE 3
HIV-1 Env binding of vaccine-induced ADCC-mediating mAbs and
blocking of sCD4 and b12 binding to Env.

| | | Binding of mAbs to HIV-1 Env | | | % Blocking by mAb | | |
|---|---|---|---|---|---|---|---|
| | | | 92TH023 | | sCD4 binding to | sCD4 binding to | b12 binding to JRFL |
| PTID¹ | mAb ID | A244 gp120 | gp120 | MN gp120 | A244 gp120 | JRFL gp120 | gp120 |
| T141485 | CH20 | −2 | ++ | ++ | − | − | − |
| T141449 | CH77 | ++ | +++ | +++ | 22 | − | − |
| | CH89 | + | ++ | ++ | − | − | − |
| | CH92 | − | − | ++ | − | − | − |
| | CH80 | + | − | ++ | 23 | − | 26 |
| | CH29 | − | − | +++ | − | − | − |
| | CH78 | ++ | +++ | ++ | 27 | 36 | 29 |
| | CH94 | +++ | +++ | +++ | − | − | − |
| | CH90 | − | − | +++ | − | − | − |
| | CH91 | ++ | +++ | +++ | − | − | − |
| T143859 | CH23 | +++ | +++ | +++ | 36 | − | − |
| 609107 | CH81 | − | − | +++ | − | − | − |
| | CH40 | +++ | ++ | +++ | 46 | 20 | 25 |
| 210884 | CH49 | +++ | − | − | − | − | − |
| | CH53 | +++ | +++ | +++ | − | − | − |
| | CH52 | +++ | +++ | +++ | 32 | − | 30 |
| | CH55 | + | − | ++ | 31 | − | 40 |
| | CH54 | + | − | +++ | − | − | − |
| | CH51 | ++ | − | +++ | − | − | − |
| 347759 | CH57 | − | +++ | +++ | − | − | − |
| | CH38 | ++ | +++ | +++ | − | − | − |
| Controls | A32 | | | | 29 | − | 23 |
| | Palivizumab | | | | − | − | − |
| | VRC-CH31 | | | | 97 | 67 | 70 |

¹PTID = Participant ID.
²+++ = IC50 <10 nM; ++ = IC50 between 10 and 100 nM; + = IC50 between 0.1 and 1 μM; − = negative/no binding/no blocking.

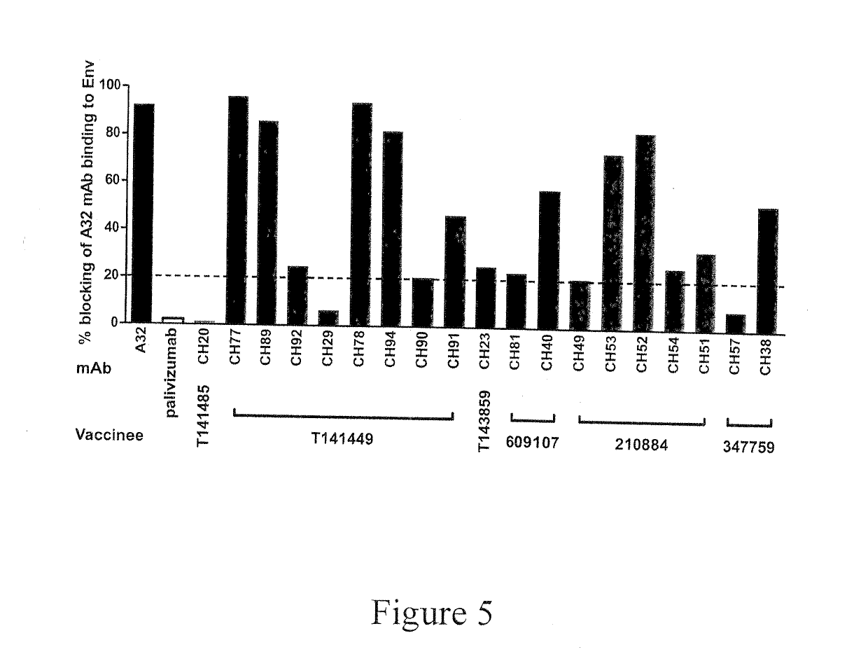

To confirm the results observed with the ADCC assay, the ability of the ADCC-mediating mAbs to block A32 binding to the AE.A244 gp120 envelope glycoprotein was tested and it was found that 16 mAbs blocked 20.7% to 94% of A32 binding to gp120 Env (FIG. 5). As expected, mAb CH20 did not block mAb A32 binding to gp120 Env, consistent with the inability of A32 Fab to block CH20-mediated ADCC activity. Of note, CH29 and CH57 did not reciprocally block A32 binding to the envelope, even though A32 Fab blocked their ADCC activity (FIG. 4) and mAb A32 blocked their binding to Env (Table 3).

It was found that 6/19 (32%) of the A32-blockable mAbs partially blocked the binding of soluble [s]CD4 and/or mAb b12 to gp120 envelope glycoproteins (Table 3). This activity ranged from 22% (CH77) to 46% (CH40) blocking of sCD4 binding to AE.A244 gp120 Env, and from 25% (CH40) to 40% (CH55) blocking of b12 binding to B.JRFL gp120 Env; in some cases blocking was higher than that seen for A32 (Table 3). These data suggest that these ADCC-mediating mAbs might interfere with binding of CD4bs-directed mAbs either by inducing conformational changes on the gp120 envelope glycoprotein or by partially blocking access to the CD4bs. The combination of blocking and binding data indicate that the ALVAC-HIV/AIDSVAX B/E vaccine induced a group of antibodies that mediate ADCC by targeting distinct but overlapping Env epitopes that are mostly A32-blockable.

Moreover, it should be noted that the original isotypes of CH29 and CH38 were IgA₁ and IgA₂, respectively (Table 2). When CH29 and CH38 were expressed as IgG₁ mAbs, they mediated ADCC activity (% GzB activities of 6.4% [CH29] and 12.4% [CH38]) that was directed against the gp120 C1 region as demonstrated by blocking with the A32 Fab (FIG. 4).

Cross-Clade ADCC Activity of RV144-Induced Antibodies.

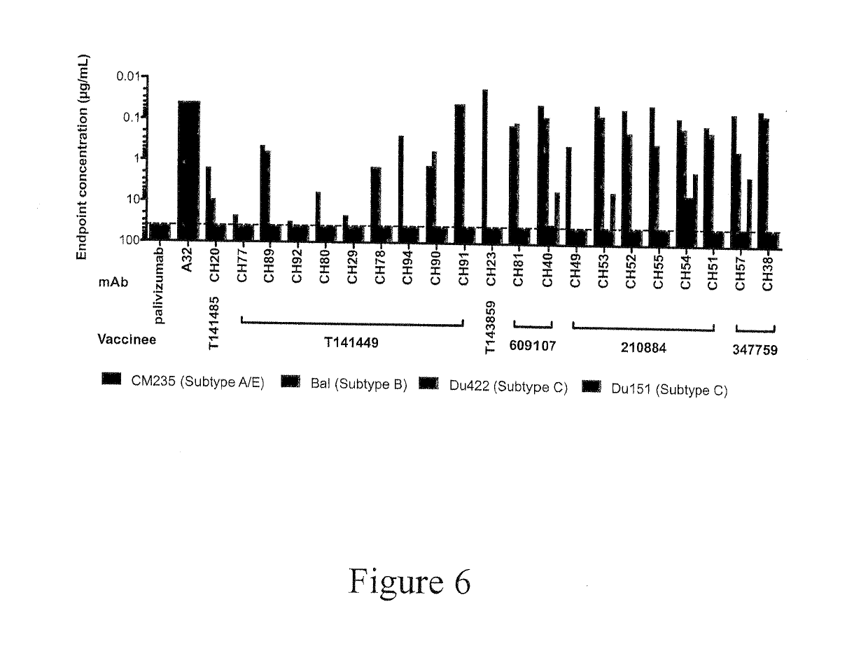

A study was next made of the ability of the 21 mAbs to mediate ADCC against viruses from different HIV-1 subtypes. Mab A32 mediated ADCC against all four tested isolates with an endpoint titer of 0.039 g/ml against all strains (FIG. 6). Each of the 21 mAbs derived from vaccines were able to mediate ADCC against target cells infected with the subtype A/E strain virus AE.CM235 while 14/21 mAbs (67%) mediated ADCC against those infected with B.Bal. When tested against subtype C virus isolates, 4/21 (19%) mediated ADCC against C.DUS51-infected target cells while a single recovered mAb (CH54) mediated ADCC against C.DU422-infected target cells (FIG. 6). The patterns of cross-clade ADCC activity, combined with the patterns observed in binding and blocking experiments, demonstrate that the RV144 immunogen elicited a diverse set of antibodies directed at epitopes overlapping, but not identical to, that of mAb A32.

VH1 Gene Family Members are Over-Represented Among ADCC-Mediating Monoclonal Antibodies Recovered from Vaccine Recipients.

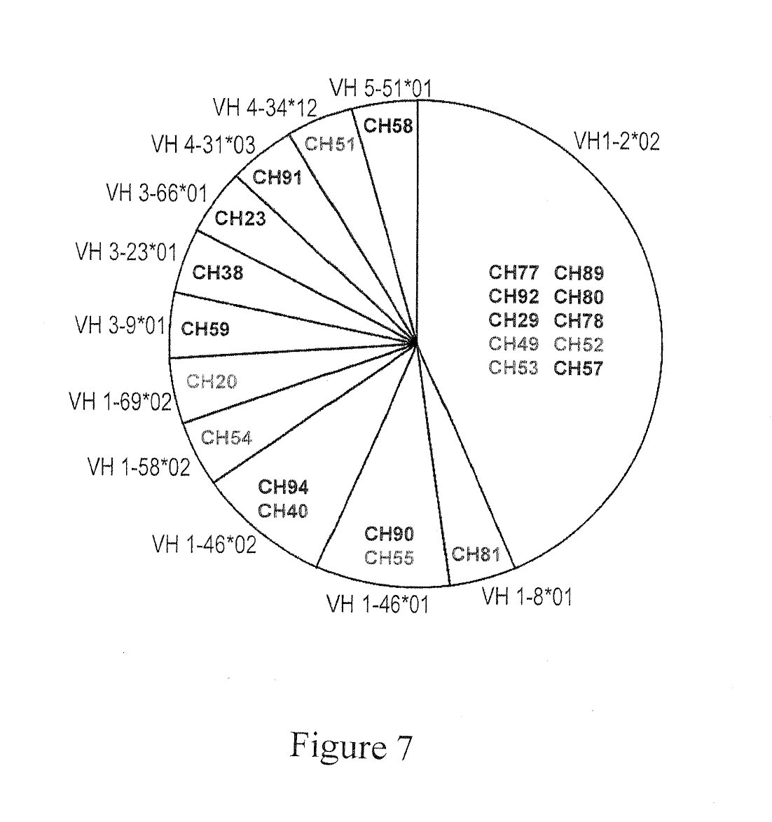

Association of anti-HIV-1 ADCC activity with the usage of a specific VH family gene has not been previously reported. It was therefore quite surprising to find that 17/23 (74%) of ADCC-mediating mAbs isolated from the vaccine recipients utilized the VH1 family gene (FIG. 7); this group includes the two anti-V2 mAbs that are described in a separate report (Liao et al, HIV-1 Envelope Antibodies Induced by ALVAC-AIDSVAX B/E Vaccine Target a Site of Vaccine Immune Pressure Within the C β-strand of gp120 V1/V2, abstr 230, p 110 Keystone Symposia—HIV Vaccines, Keystone, Colo., Mar. 21-26, 2012), which did not use VH1. In contrast, only 19/111 (17.1%) heavy chains isolated from memory B cell cultures that did not mediate ADCC used VH1 family gene segments. The frequency of VH1 family gene usage was significantly lower than for the 23 ADCC-mediating antibodies (Fisher's exact test, p<0.0001) demonstrating that the high frequency of VH1 gene usage among ADCC-mediating mAbs was not reflective of a disproportionate use of VH1 among recovered antibodies from vaccines.

The frequency of VH1 gene usage among vaccine-induced HIV-specific ADCC-mediating antibodies was higher also in comparison with other published datasets: in HIV-1 negative subjects, Brezinscheck and colleagues reported the frequency of VH1 genes to be approximately 13% (9/71 reported in (Brezinschek et al, J. Immunol. 155:190-202 (1995)); Fisher's exact test comparing the ADCC-mediating antibodies, p<0.0001), while in chronically HIV-1 infected subjects the frequency of VH1 usage in anti-HIV-1 antibodies was reported to be 39% (76/193 reported in (Breden et al, PLoS ONE 6:e16857 (2011)); Fisher's exact test comparing the ADCC-mediating antibodies, p=0.003). Frequencies of HIV-1 reactive antibodies using VH1 gene segments of 16.4% (11/67) in HIV-1 acutely infected subjects have recently been reported—which is similar to VH1 usage reported in the National Center for Biotechnology Information database (15.2%; 5,238/34,384)—(Liao et al, J. Exp. Med. 208:2237-2249 (2011)), and 38.2% (13/34) in vaccine-recipients enrolled in an unrelated HIV-1 vaccine trial (Moody et al, J. Virol. 86:7496-7507 (2012)); in both cases the frequency of VH1 gene segments usage in ALVAC-HIV/AIDSVAX B/E-induced ADCC-mediating antibodies was significantly higher (Fisher's exact test: p<0.0001 and p=0.014, respectively). In the present study, none of the recovered ADCC antibodies were clonally related, and VH1 antibodies were recovered from 5/6 vaccines studied. Thus, the high frequency of usage of VH1 heavy chain genes among antibodies that mediate ADCC suggests that B cells using those genes may have been preferentially selected by the vaccine trial Envs.

It is possible that this phenomenon may relate to properties of gp120 more generally. Analysis of a different HIV-1 vaccine trial resulted in the recovery 13/34 (38%) mAbs that used VH1 genes including 2 mAbs with ADCC activity and 1 with neutralizing activity (Moody M A et al submitted). In contrast, only 12/252 (5%) of influenza-specific antibodies recovered after influenza immunization (Moody et al, PLoS One 6:e25797 (2011)) used VH1 genes.

ADCC Activity of Antibodies Using VH Genes Correlated with the Degree of Somatic Mutation.

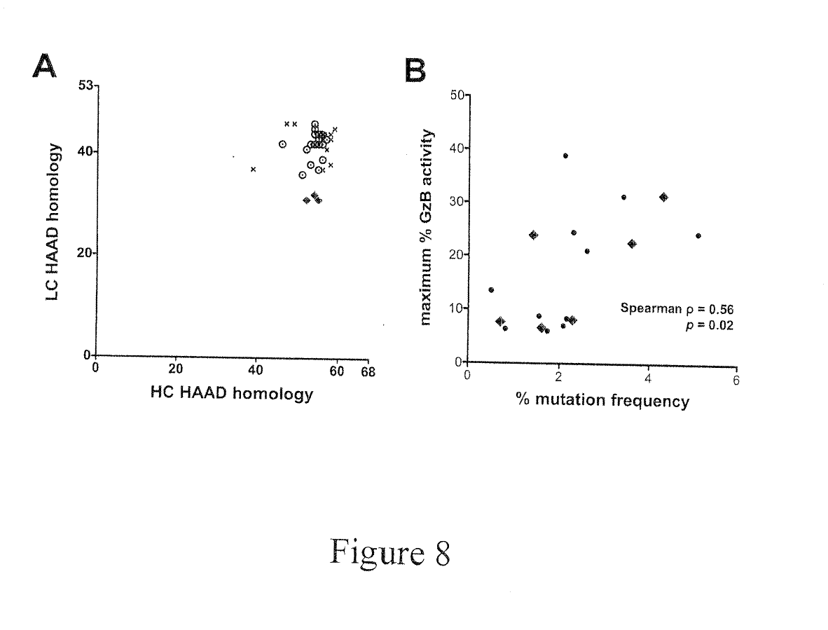

A number of recent studies have suggested that highly somatically mutated anti-CD4bs bNAb preferentially use the VH1 gene, in particular the VH1-2*02 and 1-46 segments, and common amino acid sequence motifs (HAAD motifs) have been described for both the heavy and light chains of such anti-CD4bs bNAbs (Scheid et al, Science 333:1633-1637 (2011), Wu et al, Science 329:856-861 (2010)). It was striking that among the ADCC-mediating VH1 antibodies that were recovered, 10/17 (59%) used the VH1-2*02 gene segment (FIG. 7). None of the mAbs recovered from this group of participants had broad neutralizing activity and of the mAbs reported here, only the V3-specific mAb CH23 (VH3-66) displayed tier 1 strain-specific neutralizing activity (Montefiori et al, J. Infect. Dis., Journal of Infectious Diseases 206(3): 431-441 (July 2012). A determination was made as to whether this group of antibodies shared the previously described HAAD motifs with the potent CD4bs bNAbs (Scheid et al, Science 333:1633-1637 (2011)). Alignments of the amino acid sequences of the 17 vaccine-induced ADCC-mediating antibodies that used VH1 with the heavy and light chain HAAD consensus motifs showed a high degree of similarity (range 46 to 57 matching aa of 68 aa for heavy chain, 68-84%; 37 to 46 matching as of 53 aa for light chain, 70-87%; FIG. 8A, red circles), which was comparable to the levels of similarity of the CD4bs bNAbs (FIG. 8A, black crosses). A group of three non-HIV-1-reactive VH1-2 anti-influenza antibodies that mediate broad influenza neutralization was analyzed (49). This showed a similar degree of heavy chain homology (52 to 55 matching aa, 76-81%), but less homology for light chain (31 to 32 matching aa, 58-60%; FIG. 8A, blue diamonds). Thus, the similarity of the RV144 vaccine-induced antibodies to the HAAD motif may not reflect functional selection, but rather may reflect similarities in Env-selection of B cells with similar heavy and light chain pairings.

Since the broadly neutralizing CD4bs antibodies are also highly mutated, a determination was made as to whether the degree of somatic mutation in the RV144-induced antibodies correlated with function. It was found that the ability to block sCD4 binding did not correlate with the degree of somatic mutation (FIG. 8B). In contrast, the overall strength of ADCC activity, as measured by maximal % GzB activity against CM235-infected CD4+ T cells, did correlate with heavy chain somatic mutation (Spearman correlation p=0.56, p=0.02; FIG. 8B).

In summary, the induction of neutralizing antibody [NAb] and cytotoxic T lymphocyte [CTL] responses are key goals for HIV-1 vaccine development. Recently, the phase III efficacy trial of the prime-boost combination of vaccines containing ALVAC-HIV and AIDSVAX B/E has offered the first evidence of vaccine-induced partial protection in humans (Rerks-Ngarm et al, N. Engl. J. Med. 361:2209-2220 (2009)). The vaccine appeared to induce NAb responses with a narrow specificity profile and minimal CD8+CTL responses (Rerks-Ngarm et al, N. Engl. J. Med. 361:2209-2220 (2009)) suggesting that non-neutralizing Ab and cellular responses other than CD8+CTL might have played a role in conferring protection.

A number of studies have suggested that ADCC may play an important role in the control of SIV and HIV-1 infection. Several studies have shown that the magnitude of ADCC Ab responses correlates inversely with virus set point in acute SIV infection in both unvaccinated macaques (Sun et al, J. Virol. 85:6906-6912 (2011)) and in vaccinated animals after challenge (Barouch et al, Nature 482:89-93 (2012), Brocca-Cofano et al, Vaccine 29:3310-3319 (2011), Flores et al, J. Immunol. 182:3718-3727 (2009), Gómez-Rom\acute{α}n et al, J. Immunol. 174:2185-2169 (2005)). In humans, ADCC-mediating Abs have been shown to protect against HIV-1 infection in mother-to-infant transmission (Ljunggren et al J. Infect. Dis. 161:198-202 (1990), Nag et al, J. Infect. Dis. 190:1970-1978 (2004)) and to correlate with both control of virus replication (Lambotte et al, Aids 23:897-906 (2009)) and lack of progression to overt disease (Baum et al, J. Immunol. 157:2168-2173 (1996)). In contrast, weakly neutralizing and non-neutralizing antibodies were shown to not protect against vaginal SHIV challenge in macaques (Burton et al, Proc. Natl. Acad. Sci. USA 108:11181-11186 (2011)).

ADCC is one of the mechanisms that might have conferred protection from infection in RV144 (Haynes, Case-control study of the RV144 trial for immune correlates: the analysis and way forward, abstr., p. AIDS Vaccine Conference, Bangkok, Thailand, Sep. 12-15, 2011). For this reason, studies were undertaken to isolate mAbs that can mediate ADCC from ALVAC-HIV/AIDSVAX B/E vaccine recipients and determine their specificity, clonality and maturation. In this study it has been demonstrated that the ALVAC-HIV/AIDSVAX B/E vaccine elicited antibodies that mediate ADCC in the majority of the vaccinated subjects, which is in line with previous observations (Haynes, Case-control study of the RV144 trial for immune correlates: the analysis and way forward, abstr., p. AIDS Vaccine Conference, Bangkok, Thailand, Sep. 12-15, 2011, Karnasuta et al, Vaccine 23:2522-2529 (2005)) and that gp120 C1 region-specific A32-like antibodies significantly contributed to the overall ADCC responses. By isolating 23 ADCC-mediating mAbs from multiple vaccine recipients, it was also demonstrated the presence of ADCC-mediating mAbs of additional specificities. In addition, it was determined that the ADCC-mediating mAbs underwent limited affinity maturation and preferentially used VH1 gene segments.

Antibody responses that mediate ADCC were directed toward A32-blockable conformational epitopes (n=19), a non A32-blockable conformational epitope (n=l), the gp120 Env V2 region (n=2) (23) and a linear epitope in the gp120 V3 region (n=1). The conformational epitope recognized by the A32 mAb is a dominant target of HIV-1-positive plasma ADCC antibodies (Ferrari et al, J. Virol. 85:7029-7036 (2011)) and A32-like mAbs are among the anti-HIV-1 CD4i Ab responses that are detected following HIV-1 transmission (Pollara et al, AIDS Res. Hum. Retroviruses 27:A-66 (2011), Robinson et al, Hum. Antibodies 14:115-121 (2005)). The identification of A32-like mAbs in vaccine recipients suggests that the gp120 epitope recognized by the A32 mAb could be an immunodominant region not just in response to natural infection but also upon vaccination. The data suggest that this A32-binding region reacts with antibodies that have a diverse binding profile, suggesting that the RV144 vaccine targeted multiple related but distinct conformational epitopes on gp120. These epitopes have been shown to be upregulated on the RV144 immunogen and to be efficiently presented by novel Env designs (Alam et al., submitted), thus it will be possible to test this vaccine strategy in future vaccine trials targeted to different HIV-1 subtypes.

In contrast to ADCC-mediating antibodies, HIV-1 bNab responses have been reported to appear an average of 2-4 years after HIV-1 transmission (Gray et al, J. Virol. 85:7719-7729 (2011), Mikell et al, PLoS Pathog. 7:e1001251 (2011), Mikell et al, PLoS Pathog. 7:e1001251 (2011), Shen et al, J. Virol. 83:3617-3625 (2010)), suggesting that different levels of Ab maturation are required to mediate ADCC and neutralizing activities. Indeed, the mutation frequencies observed in the mAbs isolated from the ALVAC-HIV/AIDSVAX B/E vaccine recipients in the study were low (0.5-5.1%) and well below the ˜6% changes in variable domain-amino acid sequences commonly seen as greater affinity for the cognate antigen is acquired (Moody et al, PLoS One 6:e25797 (2011), Wrammert et al, Nature 453:667-671 (2008)). It was, however, found that higher degrees of VH somatic mutation correlated with greater maximal % GzB activity (FIG. 8B) consistent with vaccine-driven affinity maturation. Whether repeated boosting of vaccine recipients would result in on-going maturation of these antibodies to further increase ADCC activity, CD4 blocking, or addition of neutralizing activity remains to be determined.

Finally, while ADCC-mediating mAbs were isolated that used diverse VH genes, a clear preferential usage of the VH1 heavy chain gene (74%) was observed, similarly to that of potent bNabs directed against the CD4bs (Scheid et al, Science 333:1633-1637 (2011), Wu et al, Science 333(6049):1593 (2011). Epub 2011 Aug. 11)). Therefore, while these findings prove that the ADCC-mediating response in these subjects was not restricted to a specific V_{H} gene family and are consistent with there being no obvious strong regulatory mechanisms that would inherently limit the generation of antibodies with ADCC activity, the preferential usage of the VH1 gene raises the hypothesis that either the Envs used in RV144 or Env gp120s in general, preferentially induce VH1 gene family use. Whether a vaccine regimen can be developed that will harness the observed Ig VH1 gene-using B cells to also induce CD4bs antibodies with a high degree of mutation is currently unknown. It is interesting to note that it was possible to recover ADCC antibodies with a degree of CD4 blocking activity that had low levels of mutation, suggesting that B cells expressing those antibodies might be harnessed to produce the desired potent CD4 blocking antibody response under the right conditions.

In conclusion, the ALVAC-HIV/AIDSVAX B/E vaccine induced potent ADCC responses mediated by modestly mutated and predominantly A32-blockable mAbs that have overlapping but distinct binding profiles. This response is qualitatively similar to anti-HIV-1 responses observed during chronic HIV-1 infections and may have been partly responsible for the modest degree of protection observed. ADCC-mediating mAbs predominantly utilized the VH1 Ig heavy chain family, which has been previously reported for CD4bs-directed broadly neutralizing antibodies. This observation raises the hypothesis that continued boosting with this vaccine formulation may lead to further somatic mutations of VH1 gp120-specific antibodies and, perhaps, to enhanced ability to augment any protective effect they might have had to limit HIV-1 acquisition.

### Example 2

### Synergy Between HIV-1 Vaccine-Elicited Envelope C1 and V2 Antibodies for Optimal Mediation of Antibody Dependent Cellular Cytotoxicity

Development of a preventive HIV-1 vaccine is a global priority. The RV144 ALVAC-prime AIDSVax-boost HIV-1 vaccine efficacy trial conducted in Thailand demonstrated an estimated 31.2% protection from infection (Rerks-Ngarm et al., 2009). An analysis of immune correlates of infection risk revealed an inverse correlation between the levels of IgG antibodies (Abs) against the HIV-1 envelope protein (Env) gp120 variable regions 1 and 2 (V1/V2) and the rate of infection (Haynes et al., 2012). A viral genetic analysis of RV144 breakthrough infections found a vaccine-induced site of immune pressure associated with vaccine efficacy at V2 amino acid position 169 (Rolland et al., 2012). Anti-V2 monoclonal antibodies (mAbs CH58 and CH59) were isolated from an RV144 vaccinee, and co-crystal structures of the mAbs and V2 peptides determined that Ab contacts centered on K169 (Liao et al., 2013). Moreover, CH58 mAb bound with the clade B gp70V1/V2 CaseA2 fusion protein used to identify V2-binding as a correlate of infection risk (Haynes et al., 2012). Mabs CH58 and CH59 do not capture or neutralize tier 2 viruses, but do bind to the surface of tier 2-HIV-1 infected CD4⁺ T cells and mediate antibody dependent cellular cytotoxicity (ADCC) (Liao et al., 2013).

Secondary immune correlates analysis of the RV144 clinical trial revealed reduced rates of infection in vaccine recipients with low levels of plasma anti-HIV-1 Env IgA Abs and high levels of ADCC activity (Haynes et al., 2012). We have previously reported that HIV-1 Env constant region 1 (C1) Ab responses constitute the dominant ADCC Ab response in RV144 vaccine recipients and have isolated several mAbs from RV144 vaccine recipients that represent this group of Ab specificities (Bonsignori et al., 2012).

The analysis of the RV144 clinical did not reveal a clear correlation between the level of anti-V2 Ab responses and a specific anti-V2 Ab function directly associated with reduced risk of infection. Based on the observation that ADCC responses that were in part mediated by anti-C mAbs may have contributed to the lower risk of infection we hypothesized that an undiscovered link may exist between vaccine-induced anti-V2 and anti-C1 Ab specificities. We sought to determine whether anti-V2 and anti-C1 Ab responses may synergize, and whether this synergy might be responsible for increased neutralizing and/or ADCC function mediated by anti-V2 Ab at concentrations similar to those observed in plasma of vaccine recipients.

Results.

Anti-V2 and Anti-C1 mAbs Isolated from RV144 Vaccine Recipients.

A summary of the characteristics of the mAbs generated from RV144 vaccine recipients (Bonsignori et al., 2012) and utilized in this study is presented in Table 4. The anti-V2 mabs CH58, CH59, HG107, and HG120 recognize Env V2 residues at positions 168-183. The CH58 and CH59 mAbs were both isolated from vaccinee 347759 and have been extensively characterized for their structural and functional properties (Liao et al., 2013). Binding profiles of CH58 suggest that this mAb best represents the anti-V2 Ab response associated with reduced rate of infection in the RV144 clinical trial (Liao et al., 2013) and was thus selected as the focus of this study. Of the 19 A32-blockable anti-C1 ADCC mAbs originally generated from the RV144 vaccine recipients (Bonsignori et al., 2012), three were of particular interest. The first, CH57 was isolated from the same vaccine recipient used to generate mAbs CH58 and CH59. The A32 Fab fragment blocked the ADCC activity of CH57, and CH57 was itself able to block binding of another RV144 ADCC mAb, CH20, that was not blocked by A32. The differential ability of CH57 and A32 to inhibit binding of CH20 suggests that they recognize overlapping, but not identical epitopes. This difference is also supported by the inability of CH57 to reciprocally block A32 in Env-binding assays. The second mAb of interest, CH54, was isolated from vaccinee 210884. CH54 displayed a similar cross-clade ADCC profile as A32, and the A32 Fab was able to block its activity. CH54 could reciprocally block 30% of A32 binding to HIV Env, but was unable to inhibit binding of CH20. Lastly, CH90 is an ADCC-mediating A32-blockable mAb generated from vaccinee T141449. This mAb blocked 20% of A32 binding, and it displayed a different cross-clade ADCC profile compared to A32. Taken together, these data suggest that CH54, CH57, and CH90 mAbs are likely recognizing distinct overlapping epitopes of the Env C1 A32-blockable region (Bonsignori et al., 2012). Therefore, they were selected as representative of vaccine-induced anti-C1 Ab responses and were tested for their ability to synergize with the anti-V2 mAb CH58 for enhanced recognition of HIV envelope and anti-viral effector functions. A32 was included to represent the overall anti-C1 Ab responses.

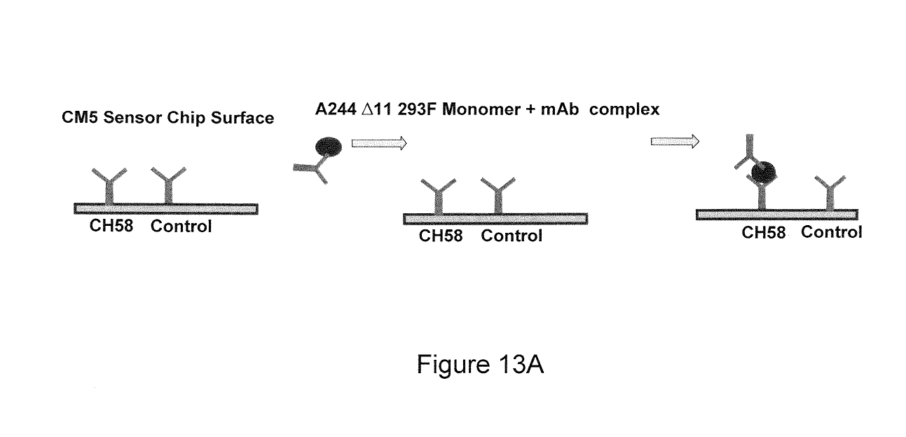

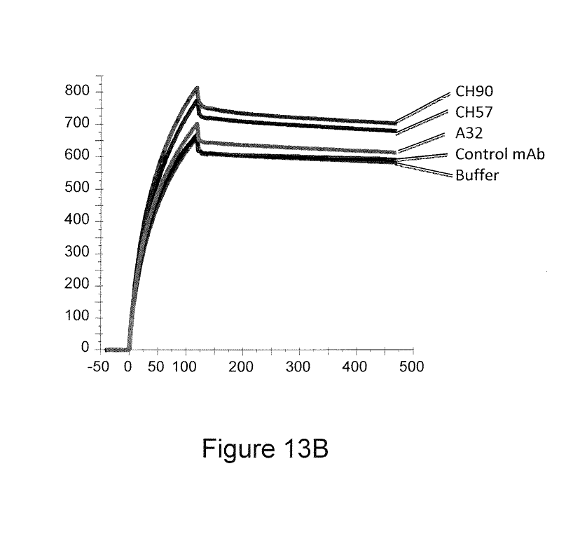

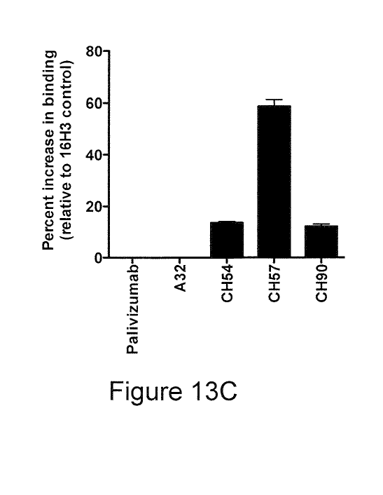

Synergy of anti-V2 and anti-C1 mAb for binding to monomeric recombinant AE.A244 Δ11 gp120. To test whether the anti-V2 CH58 mAb could synergize with the A32-blockable C1 mAbs we performed SPR analysis of binding of CH58 mAb to the recombinant AE.A244 Δ11 gp120 as representative of the vaccines used in the RV144 clinical trial. As described in the methods and displayed in FIG. 13A, the CH58 mAb was bound to the CM5 sensor chip along with the Palivizumab mAb as a negative control. The A32, CH54, CH57, and CH90 mAbs were incubated with the gp120. The capture of the mAbs-gp120 complex by the CH58 was measured by SPR. The binding curve of the anti-C1-gp120 complex to CH58 is reported in FIG. 13B. In FIG. 13C the data are expressed as % increase in binding relative to the binding of gp120 in complex with murine 16H3 mAb used as negative control. No increase in binding of mAb CH58 was observed when tested in combination with the RSV-specific negative control mAb Palivizumab, or with the anti-C1 mAb A32. In contrast, RV144 vaccinee-induced mAbs CH54, CH57, and CH90 increased the binding of mAb CH58 to recombinant HIV-1 gp120 14%, 59%, and 12%, respectively. Based on these observations, we next evaluated whether anti-V2 and anti-C1 mAbs can act in synergy for the recognition of HIV-1 infected cells.

Synergy of Anti-V2 and Anti-C1 mAb for Binding to Env Expressed on the Surface of HIV-1-Infected CD4⁺ T Cells.

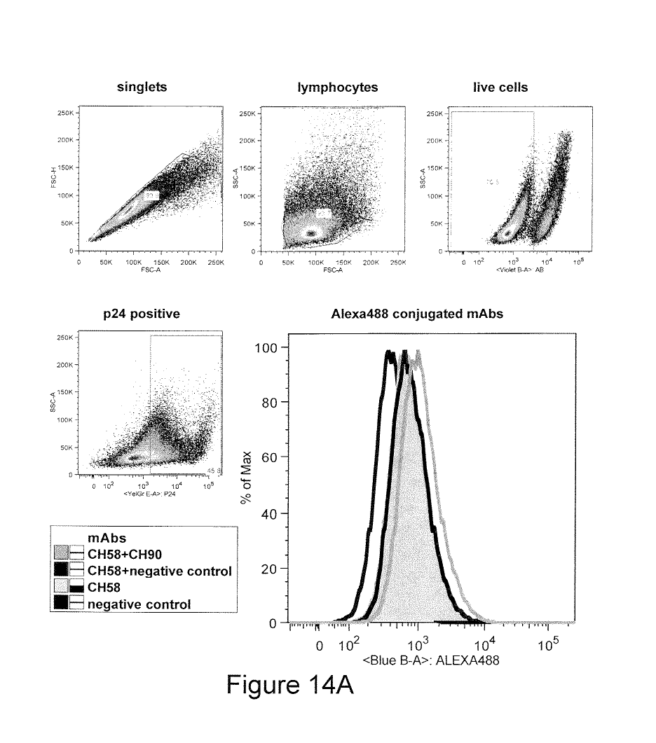

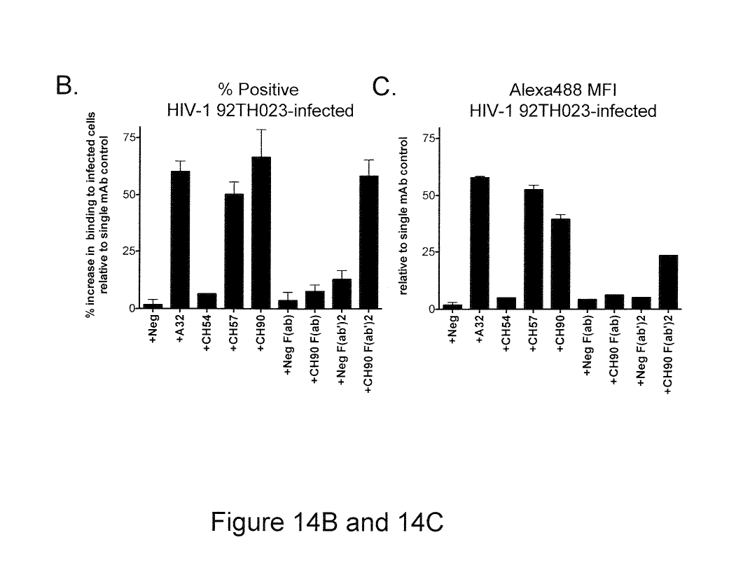

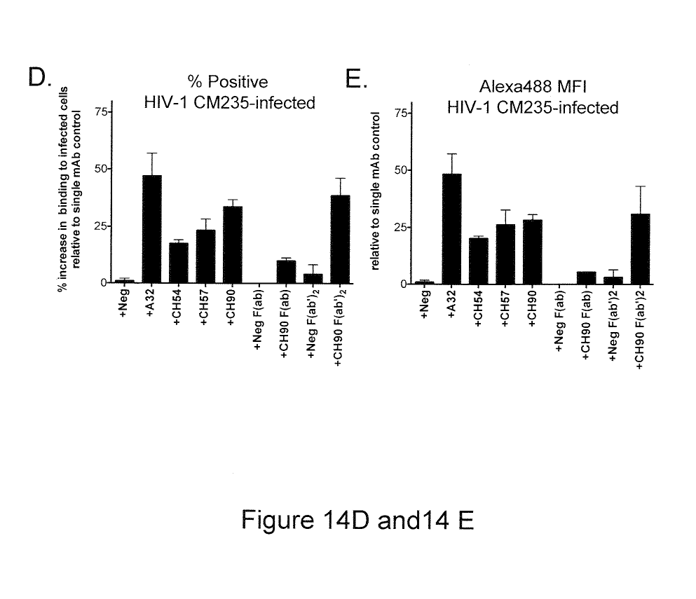

Activated primary CD4⁺ T cells isolated from a HIV-seronegative donor were infected with HIV-1 subtypes AE 92TH023 and CM235 representing a tier 1 and 2 isolate for neutralization sensitivity, respectively. The anti-V2 mAb CH58 was conjugated with Alexa Fluor@488 allowing for direct flow cytometric analysis of its ability to recognize Env on the surface of the infected cells. Co-incubation with unconjugated anti-C1 mAbs (10 μg/ml each) was used to identify binding synergy. The gating strategy used to identify live HIV-infected cells (intracellular p24⁺), and representative histograms of CH58 surface staining and CH90-induced synergy are shown in FIG. 14A. The incubation of directly conjugated CH58 mAb with AE.92TH023-infected CD4⁺ T cells in combination with the unconjugated non-fluorescent A32, CH57, and CH90 mAbs resulted in a >40% increase in the frequency of cells recognized by the CH58 mAb compared to the frequency of infected cells recognized by CH58 mAb alone (FIG. 14B). The mean fluorescence intensity of the CH58-stained cells was concomitantly increased (FIG. 14C). In contrast, we did not observe an increase in binding of CH58 to 92TH023-infected cells in the presence of mAb CH54. The incubation of AE.CM235-infected cells (FIGS. 14D and 14E) with CH58 in presence of A32 revealed a similar (<45%) increase in both the frequency of infected cells recognized by CH58, and mean fluorescence intensity of the cells. Modest enhancement of CH58 binding to CM235-infected cells was also observed with CH54 (>20% increase), CH57 (>25% increase) and CH90 (>35% increase). Collectively, these data demonstrate that anti-C1 Abs can enhance the binding of anti-V2 Abs to HIV-infected cells. However, the discordant lack of synergy between CH54 and CH58 with the AE.92TH023-infected cells compared to CM235-infected cells suggests that there are likely structural differences in the envelopes of these two HIV-1 isolates that influence the ability of Abs to synergize in the recognition of infected cells. This is further evident by the discordance between the synergy observed with CH58 and A32 in binding HIV-infected cells and lack of synergy between these mAbs in binding to A244 Δ11 gp120 monomer.

We next utilized F(ab) and F(ab′)₂ fragments of mAb CH90 to determine if synergy for binding HIV-1 infected cells was mediated by events associated strictly with interactions between the Env epitope and the Ab antigen-binding regions (Fab), or if complete Abs with class-defining region (Fc) are required. Interestingly, almost no enhancement of binding (<10%) was observed for CH58 in the presence of CH90 F(ab). However, binding of CH58 was increased in the presence of CH90 F(ab′)₂ to levels comparable to those observed with un-fragmented CH90 IgG (FIG. 14B-E). These data suggest that the Fc portion of mAb CH90 is dispensable for synergy in the recognition of infected cells with mAb CH58, but bivalent binding of the complete hinged antigen-binding region of anti-C1 mAb CH90 is necessary to induce the molecular changes that facilitate improved recognition by the anti-V2 mAb CH58.

Virion Capture Assay.

We next investigated whether the anti-C1 and anti-V2 Abs can synergize for the capture of infectious virions. Anti-V2 mAb CH58 was mixed with the AE.92TH023 HIV-1 viral stock with or without anti-C1 mAb A32 at an equimolar concentration. The mixture was absorbed by protein G-coated plates, and the capture of total and infectious virus was quantified as described in the description of the assay methodology. We did not observe any ability of CH58 to capture infectious virions, and there was no synergy in infectious virion capture between mAbs A32 and CH58.

Synergy of Anti-V2 and Anti-C1 mAbs for HIV-1 Neutralization.

The ability of anti-C1 A32 and anti-V2 mAbs to synergize in the neutralization of HIV-1 was investigated against a panel of viruses that represented HIV-1 tier 1 (B.MN, C.TV-1, AE.92TH023), tier 2 (AE.CM244), and subtype AE transmitted/founder isolates using the standard TZM-bl neutralization assay. The anti-C1 mAb A32 did not display any significant neutralizing activity when tested alone against any of the HIV-1 isolates as previously reported (Moore et al., 1995). The 50% inhibition concentration of mAbs CH58 and CH59 against the tier 1 HIV-1 AE.92TH023 isolate was 25.96 and 5.75 μg/ml, respectively. In contrast, when the two mAbs were tested in combination with the anti-C1 A32 mAb, their IC₅₀ increased 78 and over 250 fold, respectively, to 0.33 and <0.023 μg/ml (Table 5).

Synergy of Anti-V2 and Anti-C1 mAbs for ADCC.

The ability of anti-C1 and anti-V2 mAbs to synergize in the recognition of HIV-infected cells suggests that that these Ab specificities may also synergize in their ability to mediate ADCC. We focused on ADCC directed against target cells infected with the HIV-1 AE.CM235 virus as this isolate represents tier 2 neutralization sensitivity. The antiviral function of Abs against tier 2 isolates may be paramount, as transmitted/founder isolates that are responsible for the vast majority of transmission events that occur through sexual contact have also been identified to be tier 2 neutralization sensitive.

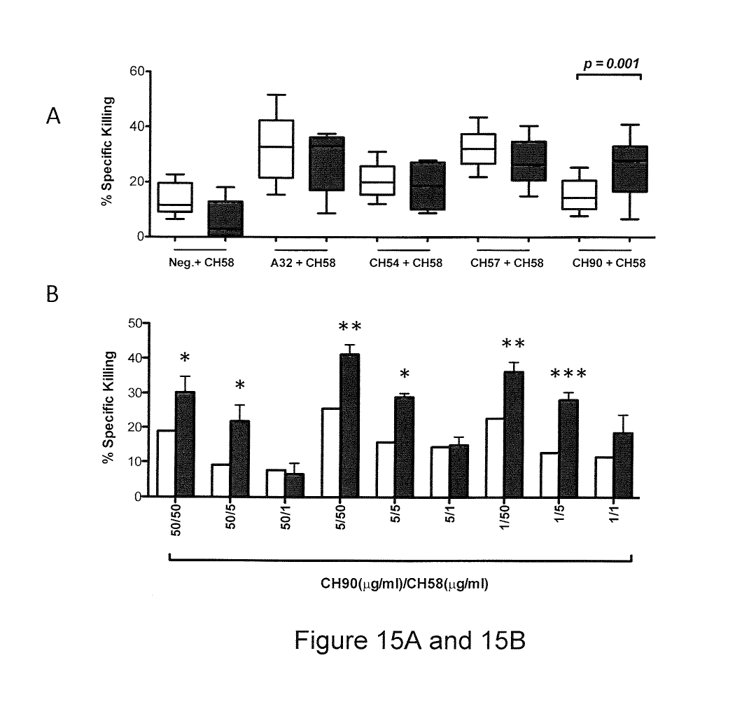

To measure ADCC, we used AE.CM23-infected CEM.NKR_{CCR5} as target cells in a 3 hour luciferase-reporter cell killing assay. The incubation time of this assay was reduced to three hours, compared to the initial description of the assay (Liao et al., 2013), to allow the detection of killing before the maximum activity of the individual mAbs is observed. Each of the vaccine-induced mAbs was tested individually at three different concentrations of 50, 5 and 1 μg/ml. The A32 mAb was tested at concentrations of 50, 1, and 0.02 μg/ml to match the potency to that of the RV144 mAbs (Bonsignori et al., 2012). To identify synergy, all combinations of the anti-C1 and CH58 anti-V2 mAb were tested. The anti-RSV mAb Palivizumab was used as negative control and its combination with CH58 represents the negative control for mAb combinations. Based on the individual testing of the mAbs, we calculated the % specific killing we would observe for an additive effect of each combination of mAbs and define this as the “expected activity” (FIGS. 15A and 15B; white bars). This parameter represents an additive effect between the two mAbs of interest. The “expected activity” was compared to the “observed” activity after the actual testing of each combination of mAbs (FIGS. 15A and 15B; filled bars).

The data presented in FIG. 15A represents the mean and interquartile range of ADCC activities for combinations of CH58 and anti-C1 mAbs across all tested concentrations of the mAb pairs indicated. ADCC synergy is evident when the observed ADCC activity of the mAb combination (filled bars) is significantly greater than that predicted by additive effect alone (white bars). As shown, there was no observable synergistic increase in ADCC activity directed against HIV-1 AE.CM235-infected target cells when CH58 was combined with Palivizumab (negative control), A32, CH54, or CH57 mAbs. However we observed a significant synergistic effect when CH58 was tested in combination with CH90 (p=0.001). The expected and observed ADCC activity of for each tested combination of CH58 and CH90 mAbs is shown in FIG. 15B. The average increase over the expected ADCC activity of CH58 and CH90 combinations was 65%, range 0%-140%.

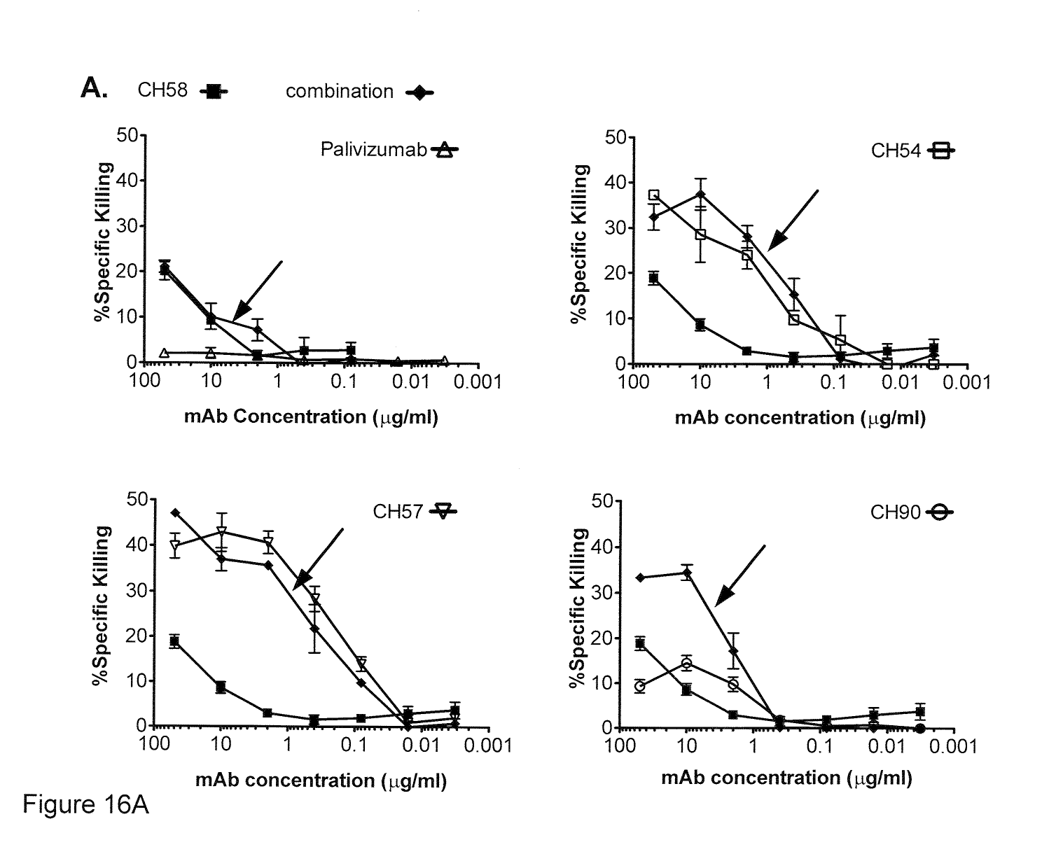

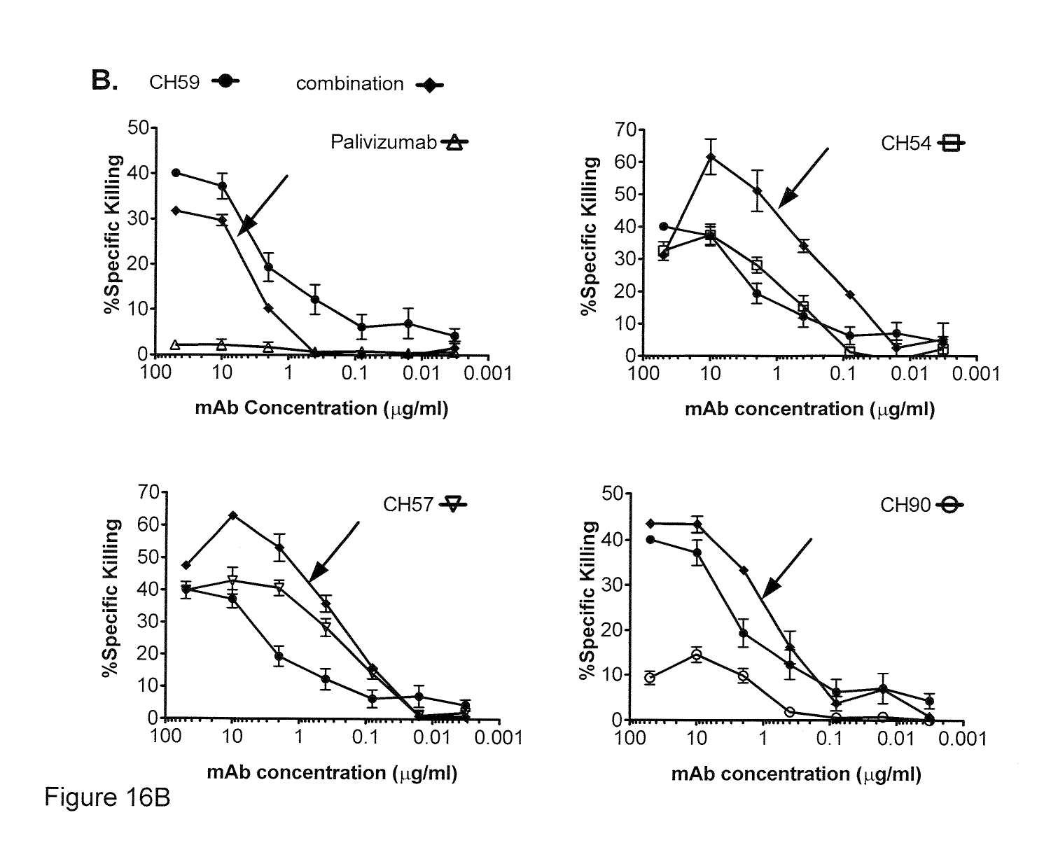

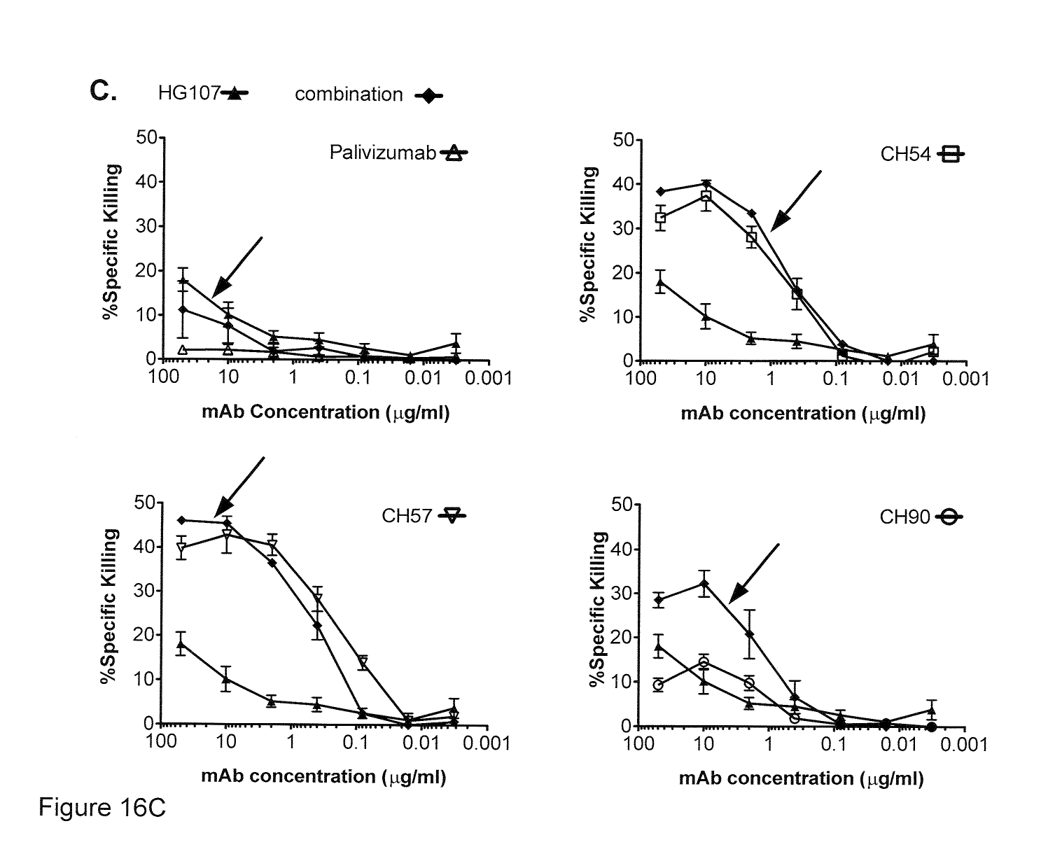

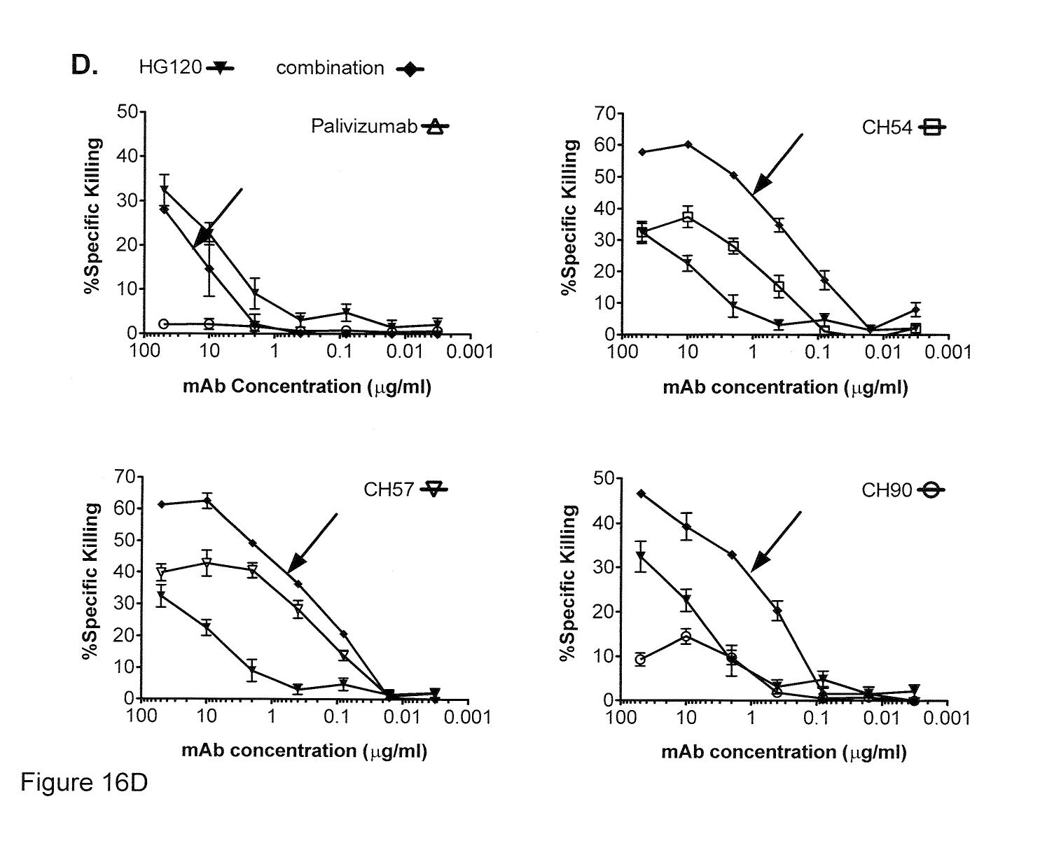

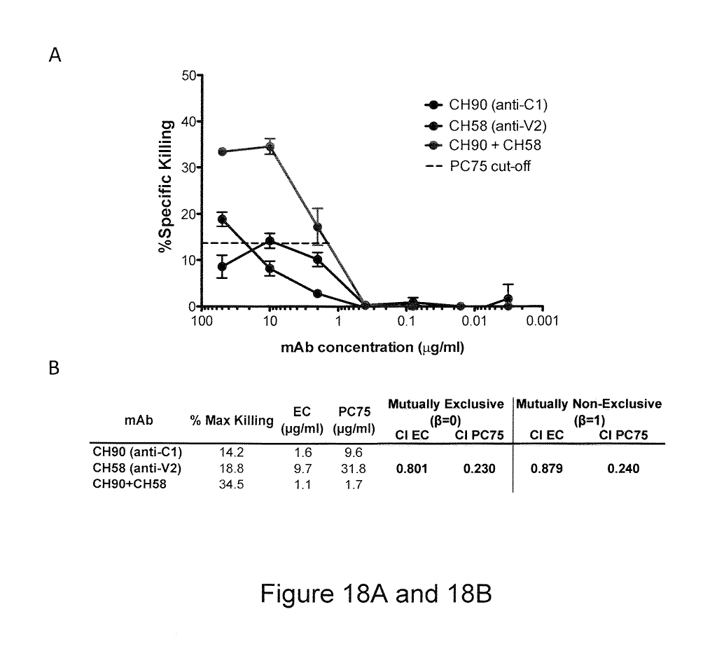

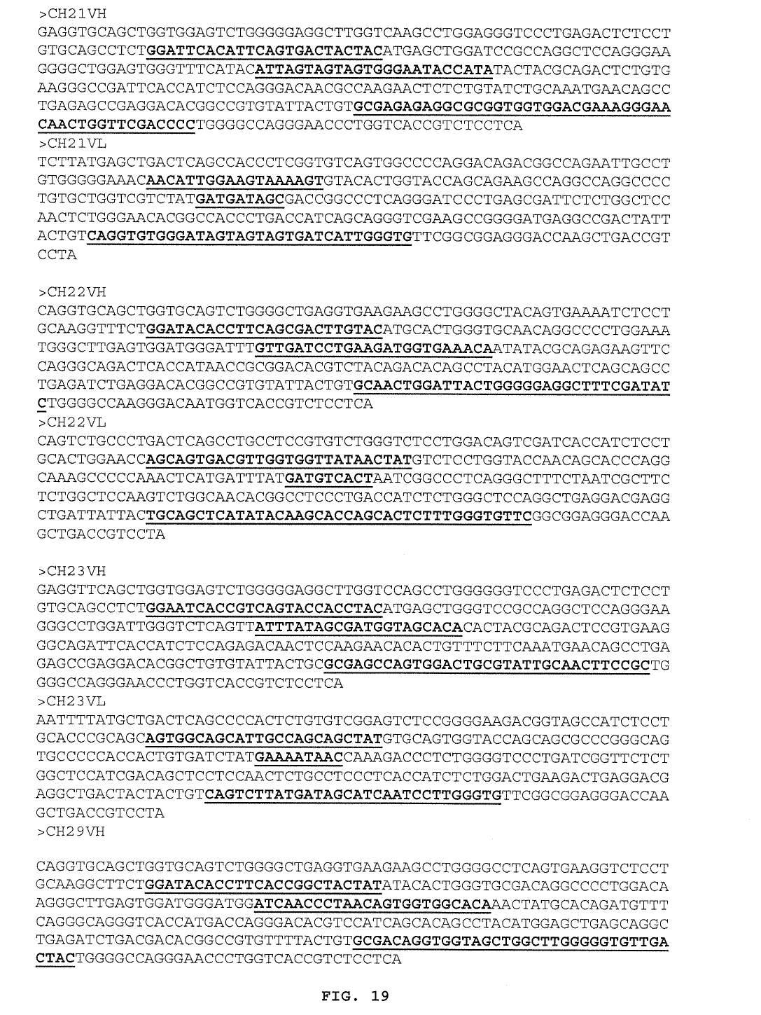

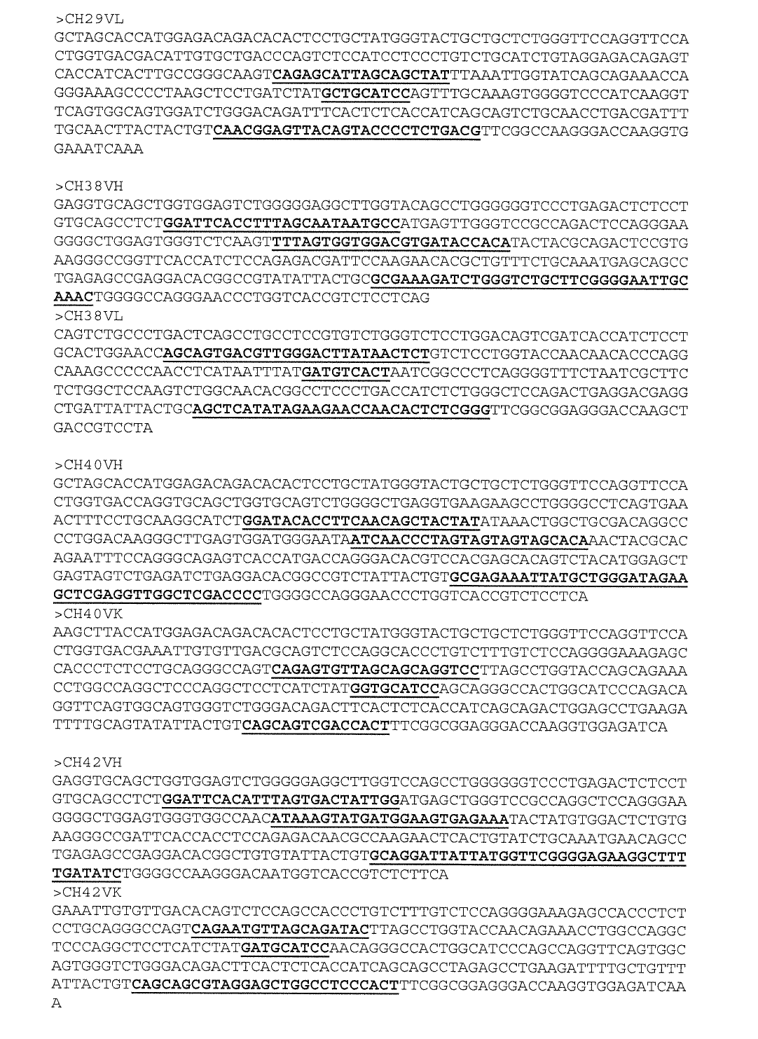

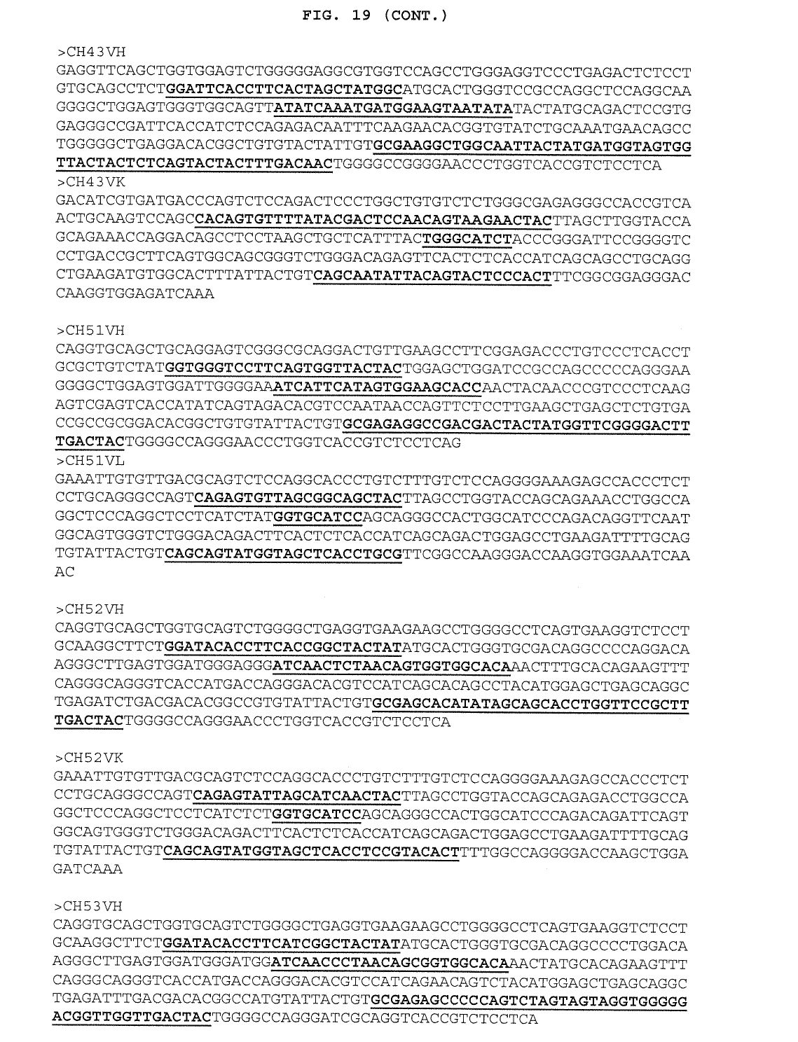

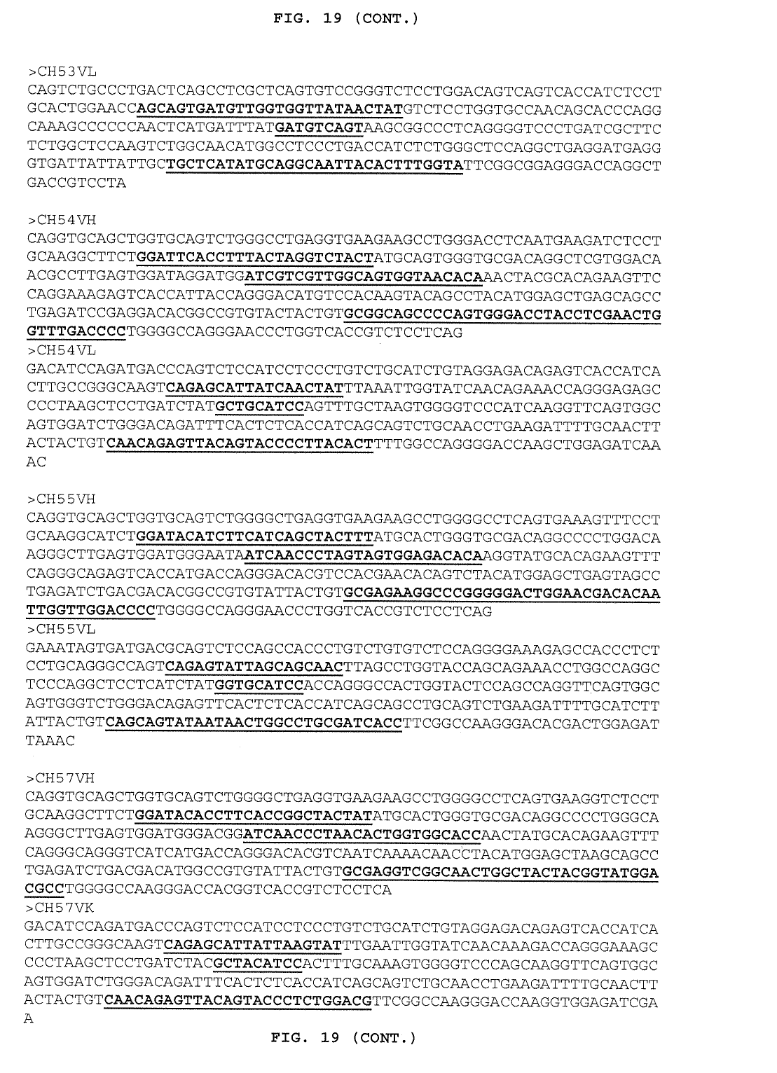

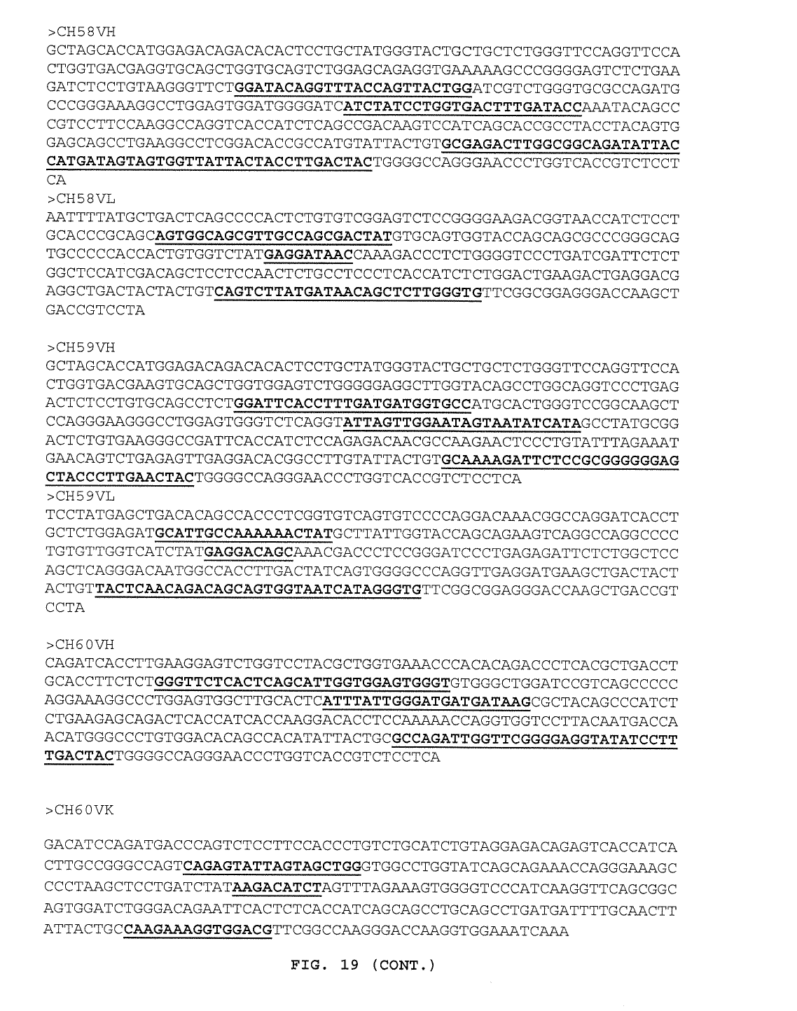

To further evaluate and quantitate synergy of anti-V2 and anti-C1 mAbs and CH90 for ADCC, we measured the activities of 5-fold serial dilutions of each antibody alone, or in equimolar combinations against AE.CM235-infected target cells (FIGS. 16, 18). Three additional RV144 vaccine recipient V2-specific mAbs, CH59, HG107 and HG120, were included in this study to more broadly characterize the potential for synergistic ADCC interactions between C1 and V2 Ab specificities. The ADCC activity curves were used to interpolate the endpoint concentration (EC) and the concentration at which 75% of the peak activity (PC75) of each mAb was reached in μg/ml. The EC and PC75 concentrations were used to calculate the combination index (CI) for the mAb pair (Table 6, 7). We have chosen to present both the mutually exclusive and non-exclusive CI values as the anti-C1 and anti-V2 mAbs recognize different regions of the HIV Env, thus fulfilling criteria of mutual exclusivity; however, they act together to mediate a single antiviral effector function (ADCC), and thus also fulfill the criteria of non-mutual exclusivity. By these methods, CI values <1 indicate a synergistic interaction, and the distance from 1 provides an indication of the magnitude of synergy. Importantly, we observed no examples of contradiction between the mutually exclusive and mutually non-exclusive methods when applied to our data set. We observed no enhancement of ADCC activity when any of the anti-V2 mAbs were tested against HIV-1 AE.CM235-infected target cells in combination with the negative control mAb, Palivizumab (FIG. 16A-D, 18) or the anti-C1 mAb A32. In contrast, most combinations of vaccine-induced anti-V2 and anti-C1 mAbs resulted in synergy for ADCC. For mAb CH58, synergy was observed only when tested in combination with anti-C1 mAb CH90 (FIGS. 16 and 18, Table 6, 7) and the degree of synergy was markedly higher for PC75 than EC. Synergy for ADCC was observed between mAb CH59 and mAbs CH54 (EC and PC75), CH57 (PC75 only), and CH90 (PC75 only) (FIGS. 16 and 18B, Table 6 and 7). For HG107, a cogent synergistic interaction was observed only when tested in combination with CH90 (FIGS. 16 and 18C, Table 6 and 7), while for HG120 strong synergy was observed with CH54, CH57, and CH90 (FIGS. 16 and 18D, Table 6 and 7). Only one anti-C1 mAb, CH90, was found to work in synergy with all four anti-V2 mAbs. As indicated in Table 6, the CI values predominately indicate a greater degree of synergy for PC75 compared to EC, which is likely a reflection of a threshold concentration of Ab needed to activate Fcγ-receptor signaling on NK effector cells.

Ab Regions Involved in ADCC Synergy.

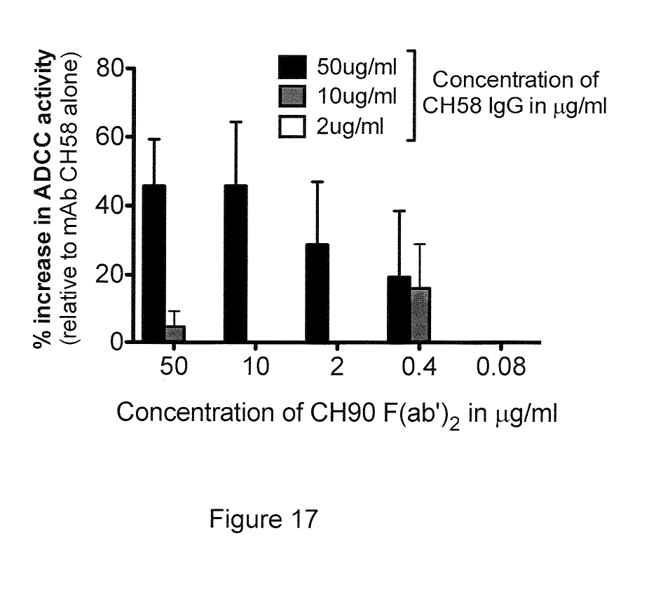

ADCC is an Ab effector function that requires two concurrent interactions: recognition of antigen by the Ab Fab region and signaling initiated by binding of the Ab Fc region with Fcγ-receptor on the surface of cytotoxic effector cells. We used the F(ab′)₂ fragment of mAb CH90 to evaluate the contribution of Fab and Fc regions to the ADCC synergy observed with mAbs CH90 and CH58. ADCC activity was measured using serial dilutions of both the CH90 F(ab′)₂ and CH58 mAb in a checkerboard matrix. As expected, the F(ab′)₂ fragment of CH90 was not able to mediate ADCC against HIV-1 AE.CM235-infected target cells. ADCC synergy was observed between the CH90 F(ab′)₂ and CH58 (FIG. 17), congruent with the observed enhancement in the recognition of HIV-1 infected cells (FIG. 14B-E). Synergy between CH90 F(ab′)₂ and CH58 was only observed at high (50 μg/ml) concentrations of CH58 mAb. ADCC synergy observed between un-fragmented mAb CH90 and mAb CH58 which was observed at all concentrations above the positive response threshold (FIGS. 16, 18). Collectively these data suggest that the synergy observed for ADCC is a consequence of both enhanced recognition of Ag on the surface of infected cells and increased recruitment and activation of ADCC effector cells.

Impact of Anti-V2/Anti-C1 Synergy on Antibody Function.

We have previously investigated the relative concentration of CH58-like Ab in the plasma of vaccine recipients using a SPR-based blocking assay. We determined that the average concentration of the vaccine-induced CH58-blockable Ab in vaccinee plasma was 3.6 g/ml±3.2 μg/ml (Liao et al., 2013). At this concentration, CH58 has no detectable neutralization or ADCC activities. The data collected in the present study indicate that anti-V2/anti-C1 synergy, as observed for the CH58/A32 and CH58/CH90 combinations, improves the binding, neutralizing, and ADCC activity of the anti-V2 CH58 mAb and reduces the required functional concentration of CH58 to levels that are plausible with those detected in the plasma of vaccine recipients.

Discussion.

In certain aspects the invention provides that anti-V2 and anti-C1 mAbs isolated from RV144 vaccines synergized for their ability to recognize Env as monomeric protein and as well, as Env expressed on the surface of HIV-1 infected cells. Moreover, both neutralizing activity against the tier 1 isolate AE.92TH023 and ADCC directed against the tier 2 HIV-1 CM235 isolate were also increased when anti-V2 antibodies were tested in the presence of anti-C1 A32-blockable antibodies.

The analysis of anti-V2 responses has revealed differences between responses induced by the vaccine regimen used in the RV144 clinical trial and natural HIV-1 infection. Anti-V2 responses were elicited in 97% of the Thai vaccine recipients whereas they have only been detected in 50% of the HIV-1 CRF01_AE-infected Thai individuals (Karasavvas et al., 2012). Moreover, the comparison of anti-V2 mAbs generated from RV144 vaccine recipients (Liao et al., 2013) to those isolated from HIV-1 infected individuals (Gorny et al., 2012) has revealed different specificities of Env V2 region recognition. In fact, CH58 CH59, HG107, and HG120 mAbs that represent the vaccine-induced anti-V2 responses recognized a linear V2 peptide comprised of amino acid residues 168-183, whereas the mAbs induced by infection recognized mainly conformational epitopes in this region (Liao et al., 2013). These differences are further supported by comparison to the canonical anti-V2 mAb, 697-D. The 697-D mAb was isolated from an HIV-infected individual, recognizes a glycosylation-dependent conformational V2 region epitope, and does not mediate ADCC (Forthal et al., 1995; Gorny et al., 1994). In direct contrast, the vaccine-elicited mAbs CH58, CH59, HG107, and HG120 CH59 recognize linear epitopes, are not affected by the presence of glycans, and are able to mediate ADCC (Liao et al., 2013).

It has recently been reported by Rolland and collaborators that the conserved presence of a lysine at the amino acid residue 169 in the V2 region was associated with vaccine efficacy using sieve analysis (Rolland et al., 2012). Liao and collaborators demonstrated that binding, neutralizing, and ADCC activity of the CH58 and CH59 were severely impacted by the mutations at position 169 observed in the transmitted/founder HIV-1 isolated from breakthrough vaccine recipients. In contrast, the activities of anti-V2 isolated from infected individuals were moderately or not at all affected by the presence of these mutations (Liao et al., 2013).

Taken together, these observations indicate that RV144 vaccine-induced anti-V2 responses are indeed different than those elicited by HIV-1 infection and may therefore have different immune effector functions.

In this study we have identified synergy between anti-V2 and anti-C1 mAbs in binding to monomeric gp120, binding to HIV-infected cells, virus neutralization, and ability to mediate ADCC. Interestingly, the profiles of synergy were different among the RV144 vaccine-induced anti-C1 mAbs, which likely reflect differences in the functional roles of the overlapping C1 epitopes recognized by these mAbs. For example, CH57 acted in synergy with CH58 in binding to both recombinant Env and to the surface of HIV-1 infected cells, but there was no observed synergy between CH57 and CH58 for ADCC. This differs from CH90, which only modestly improved binding of CH58 to gp120, but more potently increased binding to HIV-infected cells and ADCC. These data suggest that improved recognition of HIV Env or HIV-infected cells does not consistently predict ADCC. This finding is in contrast to a previous study that described a direct correlation between the ability of Env-specific polyclonal IgG to bind to infected cells and to mediate ADCC (Smalls-Mantey et al., 2012). It is therefore likely that polyclonal IgG preparations reflect a repertoire of antigen specificities that was not recapitulated by our study on mAbs with limited specificities. In the absence of a well defined binding site for A32 and the three anti-C1 RV144 mAbs we cannot fully define which interactions may exist or be required to increase both the binding to Env and the anti-viral functions of the anti-V2 mAb CH58. Furthermore, differences observed between synergistic binding to the surface of cells infected with the tier 1 HIV isolate AE.92TH023 and the tier 2 isolate AE.CM235 as demonstrated for the combination of CH58 and CH54 suggest that synergy is finely influenced by both the original conformational structures of the epitopes recognized by the combination of mAbs, and structural differences between envelopes of the HIV isolates. Additional structural studies will need to be performed to resolve the fine details of these molecular interactions.

We utilized mAb F(ab) and F(ab′)₂ fragments to identify Ab regions involved in binding synergy and ADCC synergy. These experiments demonstrated that F(ab′)₂, but not F(ab) fragments were sufficient to induce the molecular changes in Env expressed on the surface of HIV-1 infected cells that allow for enhanced recognition by mAb CH58. Using F(ab′)₂ fragments we also determined that the ability of these non-Fc bearing fragments to enhance binding can result in ADCC synergy at high concentrations of mAb. However, augmented Fey-receptor and Ab Fc interactions that are likely facilitated by multivalent recognition of Env when un-fragmented anti-C1 and anti-V2 mAbs were used in combination resulted in the most potent synergy. To our knowledge, this study is the first study to demonstrate that anti-HIV Ab synergy occurs at both the levels of Ag recognition and effector cell recruitment.

Importantly, synergy for ADCC was observed for most combinations of anti-C1 and anti-V2 mAbs against the tier 2 neutralization sensitive isolate AE.CM235. Transmitted founder viruses isolated from infected vaccine-recipients are also tier 2 sensitive and therefore our findings support the hypothesis that these types of synergistic interactions could be indeed related to the ability of the immune system to reduce the risk of infection as observed in the RV144 vaccine trial. Moreover, we observed that the anti-V2/anti-C synergistic activity was ultimately capable of increasing the CH58 mAb neutralizing and ADCC functions at concentrations of CH58 mAb that are lower than the average concentrations CH58-like antibodies detected in the plasma of RV144 vaccine recipients.

Overall, our observations indicate for the first time that synergistic mechanisms of action exist for functional non-neutralizing Ab responses correlated to the reduced of risk of HIV-1 infection. These synergistic interactions should be further explored following passive and active immunization studies to understand the regions of Env that may need to be targeted by the future generation of AIDS vaccine.

Methods.

Plasma and Cellular Samples from Vaccine Recipients.

Plasma samples were obtained from volunteers receiving the prime-boost combination of vaccines containing ALVAC-HIV (vCP1521) (Sanofi Pasteur) and AIDSVAX B/E (Global Solutions for Infectious Diseases). Vaccine recipients were enrolled in the Phase I/II clinical trial (Nitayaphan et al., 2004) and in the community-based, randomized, multicenter, double-blind, placebo-controlled phase III efficacy trial (Rerks-Ngarm et al., 2009).

Peripheral blood mononuclear cells (PBMCs) from five HIV-1 uninfected vaccine recipients enrolled in the phase II (recipient T141449) and phase III (recipients 347759, 210884, 200134, and 302689) trials whose plasma showed ADCC activity were used for isolation of memory B cells and production of monoclonal antibodies (mAbs).

All trial participants gave written informed consent as described for both studies. Samples were collected and tested according to protocols approved by Institutional Review Boards at each site involved in these studies.

Isolation of ADCC-Mediating Monoclonal Antibodies.

Monoclonal antibodies were isolated from subjects 210884 (CH54), 347759 (CH57, CH58, and CH59) and 200134 (HG107) by culturing IgG⁺ memory B cells at near clonal dilution for 14 days (Bonsignori et al., 2011) followed by sequential screenings of culture supernatants for HIV-1 gp120 Env binding and ADCC activity as previously reported (Bonsignori et al., 2012). The mAbs CH90 and HG120 were isolated from subjects T141449 and 302689, respectively, by flow cytometry sorting of memory B cells that bound to HIV-1 group M consensus gp140_{Con.S} Env as previously described (Gray et al., 2011) and with subsequent modification (Bonsignori et al., 2012).

Generation of mAb F(Ab) and F(Ab)₂ Fragments.

F(ab) and F(ab′)₂ fragments were produced by papain or pepsin digestion, respectively, of recombinant IgG1 mAbs using specific fragment preparation kits (Pierce Protein Biology Products) according to the manufactures instructions. The resulting fragments were characterized by SDS-PAGE under reducing and non-reducing conditions and by FPLC.

Surface Plasmon Resonance (SPR) Kinetics and Dissociation Constant (K_{d}) Measurements.

Env gp120 binding K_{d} and rate constant for IgG mAbs were calculated on BIAcore 3000 instruments using an anti-human Ig Fc capture assay as described earlier (Alam et al., 2007; 2008). The humanized monoclonal antibody (IgG1ₖ) directed to an epitope in the A antigenic site of the F protein of respiratory syncytial virus, Palivizumab (MedImmune, LLC; Gaithersburg, Md.), was purchased from the manufacturer and used as a negative control. Palivizumab was captured on the same sensor chip as a control surface. Non-specific binding of Env gp120 to the control surface and/or blank buffer flow was subtracted for each mAb-gp120 binding interactions. All curve fitting analyses were performed using global fit of multiple titrations to the 1:1 Langmuir model. Mean and standard deviation (s.d.) of rate constants and K_{d} were calculated from at least three measurements on individual sensor surfaces with equivalent amounts of captured antibody. All data analysis was performed using the BIAevaluation 4.1 analysis software (GE Healthcare).

SPR Antibody Synergy Assay.

SPR antibody synergy of monoclonal antibody binding was measured on BIAcore 4000 instruments by immobilizing the test anti-V2 mAb (IgG) on a CM5 sensor chip to about 5,000-6,000 RU using standard amine coupling chemistry. Anti-C1 mAbs (A32, CH57. CH90, 16H3) at 40 ug/mL were pre-incubated with Env gp120 (20 ug/mL) in solution and then injected over CH58 immobilized surface. Env gp120-mAb complexes were injected at 10 uL/min for 2 min and the dissociation monitored for 5 mins. Following each binding cycle, surfaces were regenerated with a short injection (10-15s) of either Glycine-HCl pH2.0. Enhancement of binding was calculated from binding responses measured in the early dissociation phase and % enhancement was calculated from the ratio of binding response as follows—[% enhancement=(1−(Response with gp120+up-regulating Ab−Response with gp120+control mAb/Response with gp120+control mAb)*100]. A schematic of this method is provided in FIG. 13.

Infectious Molecular Clones (IMC).

HIV-1 reporter virus used was a replication-competent infectious molecular clone (IMC) designed to encode the CM235 (subtype A/E) env genes in cis within an isogenic backbone that also expresses the Renilla luciferase reporter gene and preserves all viral open reading frames (Edmonds et al., 2010). The Env-IMC-LucR virus used was the NL-LucR.T2A-AE.CM235-ecto (IMC_{CM235}) (GenBank No. AF259954.1; plasmid provided by Dr. Jerome Kim, US Military HIV Research Program). Reporter virus stocks were generated by transfection of 293T cells with proviral IMC plasmid DNA, and virus titer was determined on TZM-bl cells for quality control (Adachi et al., 1986).

Infection of CEM.NKR_{CCR5} Cell Line and Primary CD4⁺ T Cells with HIV-1 IMC.

Primary CD4+ T cells used in surface staining assays were activated, isolated, and infected with uncloned HIV-1 92TH023 virus or IMC_{CM235} by spinoculation as previously described (Ferrari et al., 2011). For ADCC assays, IMC_{CM235} was titrated in order to achieve maximum expression within 36 hours post-infection as determined by detection of Luciferase activity and intra-cellular p24 expression. We infected 1×10⁶ cells with 1 TCID50/cell IMC_{CM235} by incubation for 0.5 hour at 37° C. and 5% CO₂ in presence of DEAE-Dextran (7.5 μg/ml). The cells were subsequently resuspended at 0.5×10⁶/ml and cultured for 36 hours in complete medium containing 7.5 μg/ml DEAE-Dextran. On ADCC assay day, the infection of target cells was monitored by measuring the frequency of cells expressing intracellular p24. The assays performed using the infected target cells were considered reliable if the percentage of viable p24⁺ target cells on assay day was ≧20%.

Binding of mAbs to the Surface of HIV-1 Infected Primary CD4⁺ T Cells.

The staining of infected CD4⁺ T cells was performed as a modification of the previously published procedure (Ferrari et al., 2011). Briefly, the A32 mAb and vaccine-induced anti-C1 A32 blockable mAbs were pre-incubated with the infected cells for 15 minutes at 37° C. in 5% CO₂ prior to addition of the vaccine induced anti-V2 mAb CH58. The anti-V2 purified mAb CH58 was conjugated to Alexa Fluor 488 (Invitrogen, Carlsbad, Calif.) using a monoclonal antibody conjugation kit per the manufacturer's instructions (Invitrogen, Carlsbad, Calif.). Both the C1-specific and V2-specific mAbs were used at a final concentration of 10 μg/ml. The combined mAbs were incubated with the infected cells for 2-3 hours at 37° C. in 5% CO₂ after which the cells were stained with a viability dye and for intracellular expression of p24 by standard methods.

Virion Capture Assay.

Anti-V2 CH58 mAb was mixed with 2×10⁷ RNA copies/mL AE.92TH023 HIV-1 viral stock at final concentration of 10 μg/ml in 300 μl with or without the presence 10 g/ml A32 antibody. The mAbs and virus immune-complex mixture were prepared in vitro and absorbed by protein G MultiTrap 96-well plate as described (Liu et al., 2011). The viral particles in the flow-through or captured fraction were measured by detection of viral RNA with HIV-1 gag real time RT-PCR. The infectious virus in the flow-through was measured by infecting the TZM-bl reporter cell line. Briefly, 25 μl flow-through was used to infect TZM-bl cells. Each sample was run in triplicate. Infection was measured by a firefly luciferase assay at 48 hours post infection as described previously. One-hundred μl of supernatant was removed and 100 μl Britelite (Perkin Elmer) were added to each well. After two minutes incubation, 150 μl of lysate was used to measure HIV-1 replication as expressed as relative luciferase units (RLUs). The percentage of viral particles in the flow-through or capture fraction was calculated as the flow-through or capture RNA/(flow-through+capture)×100%. The percentage of captured infectivity was calculated as 100−the flow-through infectivity/Virus only infectivity×100%.

Neutralization Assays.

Neutralizing antibody assays in TZM-bl cells were performed as described previously (Montefiori, 2001). Neutralizing activity of anti-V2 CH58 and CH59 in serial three-fold dilutions starting at 50 μg/ml final concentration was tested against 5 pseudotyped HIV-1 viruses including tier 1 and tier 2 B.MN, AE.92TH023 and tier 2 AE.CM244, from which RV144 vaccine immunogens (Rerks-Ngarm et al., 2009) were derived from, as well the transmitted/founder AE.427299 and AE.703357 HIV-1 isolated from breakthrough HIV-1 infected RV144 vaccine recipients. Each mAb was tested alone or in combination with A32 mAb at concentrations of 50, 25, or 5 μg/ml. The data were calculated as a reduction in luminescence compared with control wells and reported as mAb IC₅₀ in μg/ml.

Luciferase ADCC Assay.

We utilized a modified version of our previously published ADCC luciferase procedure (Liao et al., 2013). Briefly, CEM.NKR_{CCR5} cells (NIH AIDS Research and Reference Reagent Repository) (Trkola et al., 1999) were used as targets for ADCC luciferase assays after infection with the AE.HIV-1 IMC_{CM235}. The target cells were incubated in the presence of 50, 5, or 1 μg/ml of vaccine-induced anti-V2 and anti-C1 mAbs. Because of its potency in ADCC assay, the dilution scheme for the A32 mAb was 50, 1, and 0.02 μg/ml. Purified CD3⁻CD16⁺ NK cells were obtained from a HIV seronegative donor with the low-affinity 158F/F Fcγ receptor IIIa phenotype (Lehrnbecher et al., 1999). The NK cells were isolated from cryopreserved PBMCs by negative selection with magnetic beads (Miltenyi Biotec GmbH, Germany) after resting overnight. The NK cells were used as effector cells at an effector to target ratio of 5:1. The effector cells, target cells, and Ab dilutions were plated in opaque 96-well half area plates and were incubated for 3 hours at 37° C. in 5% CO₂. The final read-out was the luminescence intensity generated by the presence of residual intact target cells that have not been lysed by the effector population in the presence of ADCC-mediating mAb. The % of killing was calculated using the formula:

$$\% killing = \frac{( RLU of Target + Effector well ) - ( RLU of test well )}{RLU of Target + Effector well} \times 100$$

In this analysis, the RLU of the target plus effector wells represents spontaneous lysis in absence of any source of Ab. The RSV-specific mAb Palivizumab was used as a negative control.

We also evaluated synergy between CH58, A32, and the RV144 anti-C1 mAbs at equivalent (1:1) concentrations across a range of 5-fold serial dilutions beginning at 50 μg/ml. From the ADCC activity curves, we interpolated the endpoint concentration (EC) in μg/ml and the concentration at which 75% of the peak activity (PC75) of CH58 mAb was reached in μg/ml. From these values we calculated the combination index (CI) as described (Chou and Talalay, 1984). For example, the CI_{EC} was calculated according to the following equation:

$${CI}_{EC} = \frac{{EC}_{( anti \text{-} C 1 , combination )}}{{EC}_{( anti \text{-} C 1 , alone )}} + \frac{{EC}_{( anti \text{-} V 2 , combination )}}{{EC}_{( anti \text{-} V 2 , alone )}} + β \times \frac{( {EC}_{( anti \text{-} C 1 , combination )} \times {EC}_{( anti \text{-} V 2 , combination )} )}{( {EC}_{( anti \text{-} C 1 , alone )} \times {EC}_{( anti \text{-} V 2 , alone )} )}$$

Where EC_{(anti-C1, alone)} and EC_{(anti-V2,alone)} are the EC in μg/ml of each mAb when tested alone, and EC_{(anti-C1 combination)} and EC_{(anti-V2, combination)} are the EC in μg/ml of the mAbs when used in combination. The same formula was used to calculate the CI_{PC75} with respective substitutions of PC75 concentrations. Both mutually exclusive (3=0) and mutually non-exclusive (3=1) CI values were determined. Synergy is indicated by CI values of <1, additivity by CI values=1, and antagonism by CI values >1.

### REFERENCES CITED IN EXAMPLE 2

Adachi, A., Gendelman, H. E., Koenig, S., Folks, T., Willey, R., Rabson, A., and Martin, M. A. (1986). Production of acquired immunodeficiency syndrome-associated retrovirus in human and nonhuman cells transfected with an infectious molecular clone. J Virol 59, 284-291.
Alam, S. M., McAdams, M., Boren, D., Rak, M., Scearce, R. M., Gao, F., Camacho, Z. T., Gewirth, D., Kelsoe, G., Chen, P., et al. (2007). The role of antibody polyspecificity and lipid reactivity in binding of broadly neutralizing anti-HIV-1 envelope human monoclonal antibodies 2F5 and 4E10 to glycoprotein 41 membrane proximal envelope epitopes. J Immunol 178, 4424-4435.
Alam, S. M., Scearce, R. M., Parks, R. J., Plonk, K., Plonk, S. G., Sutherland, L. L., Gomy, M. K., Zolla-Pazner, S., Vanleeuwen, S., Moody, M. A., et al. (2008). Human immunodeficiency virus type 1 gp41 antibodies that mask membrane proximal region epitopes: antibody binding kinetics, induction, and potential for regulation in acute infection. J Virol 82, 115-125.
Bonsignori, M., Hwang, K.-K., Chen, X., Tsao, C.-Y., Morris, L., Gray, E., Marshall, D. J., Crump, J. A., Kapiga, S. H., Sam, N. E., et al. (2011). Analysis of a clonal lineage of HIV-1 envelope V2/V3 conformational epitope-specific broadly neutralizing antibodies and their inferred unmutated common ancestors. J Virol 85, 9998-10009.
Bonsignori, M., Pollara, J., Moody, M. A., Alpert, M. D., Chen, X., Hwang, K.-K., Gilbert, P. B., Huang, Y., Gurley, T. C., Kozink, D. M., et al. (2012). Antibody-Dependent Cellular Cytotoxicity-Mediating Antibodies from an HIV-1 Vaccine Efficacy Trial Target Multiple Epitopes and Preferentially Use the VH1 Gene Family. J Virol 86, 11521-11532.
Chou, T.-C., and Talalay, P. (1984). Quantitative analysis of dose-effect relationships: the combined effects of multiple drugs or enzyme inhibitors. Advances in Enzyme Regulation 22, 27-55.
Edmonds, T. G., Ding, H., Yuan, X., Wei, Q., Smith, K. S., Conway, J. A., Wieczorek, L., Brown, B., Polonis, V., West, J. T., et al. (2010). Replication competent molecular clones of HIV-1 expressing Renilla luciferase facilitate the analysis of antibody inhibition in PBMC. Virology 408, 1-13.
Ferrari, G., Pollara, J., Kozink, D., Harms, T., Drinker, M., Freel, S., Moody, M. A., Alam, S. M., Tomaras, G. D., Ochsenbauer, C., et al. (2011). An HIV-1 gp120 envelope human monoclonal antibody that recognizes a C1 conformational epitope mediates potent antibody-dependent cellular cytotoxicity (ADCC) activity and defines a common ADCC epitope in human HIV-1 serum. J Virol 85, 7029-7036.
Forthal, D. N., Landucci, G., Gorny, M. K., Zolla-Pazner, S., and Robinson, W. E. (1995). Functional activities of 20 human immunodeficiency virus type 1 (HIV-1)-specific human monoclonal antibodies. AIDS Res Hum Retroviruses 11, 1095-1099.
Gorny, M. K., Moore, J. P., Conley, A. J., Karwowska, S., Sodroski, J., Williams, C., Burda, S., Boots, L. J., and Zolla-Pazner, S. (1994). Human anti-V2 monoclonal antibody that neutralizes primary but not laboratory isolates of human immunodeficiency virus type 1. J Virol 68, 8312-8320.
Gorny, M. K., Pan, R., Williams, C., Wang, X.-H., Volsky, B., O'Neal, T., Spurrier, B., Sampson, J. M., Li, L., Seaman, M. S., et al. (2012). Functional and immunochemical cross-reactivity of V2-specific monoclonal antibodies from HIV-1-infected individuals. Virology 427, 198-207.
Gray, E. S., Moody, M. A., Wibmer, C. K., Chen, X., Marshall, D., Amos, J., Moore, P. L., Foulger, A., Yu, J.-S., Lambson, B., et al. (2011). Isolation of a monoclonal antibody that targets the alpha-2 helix of gp120 and represents the initial autologous neutralizing-antibody response in an HIV-1 subtype C-infected individual. J Virol 85, 7719-7729.
Haynes, B. F., Gilbert, P. B., McElrath, M. J., Zolla-Pazner, S., Tomaras, G. D., Alam, S. M., Evans, D. T., Montefiori, D. C., Karnasuta, C., Sutthent, R., et al. (2012). Immune-correlates analysis of an HIV-1 vaccine efficacy trial. N Engl J Med 366, 1275-1286.
Karasavvas, N., Billings, E., Rao, M., Williams, C., Zolla-Pazner, S., Bailer. R.T., Koup, R. A., Madnote, S., Arworn, D., Shen, X., et al. (2012). The Thai Phase III HIV Type 1 Vaccine Trial (RV144) Regimen Induces Antibodies That Target Conserved Regions Within the V2 Loop of gp120. AIDS Res Hum Retroviruses 28, 1444-1457.
Lehrnbecher, T., Foster, C. B., Zhu, S., Leitman, S. F., Goldin, L. R., Huppi, K., and Chanock, S. J. (1999). Variant genotypes of the low-affinity Fcgamma receptors in two control populations and a review of low-affinity Fcgamma receptor polymorphisms in control and disease populations. Blood 94, 4220-4232.
Liao, H.-X., Bonsignori, M., Alam, S. M., McLellan, J. S., Tomaras, G. D., Moody, M. A., Kozink, D. M., Hwang, K.-K., Chen, X., Tsao, C.-Y., et al. (2013). Vaccine Induction of Antibodies against a Structurally Heterogeneous Site of Immune Pressure within HIV-1 Envelope Protein Variable Regions 1 and 2. Immunity 38, 176-186.
Liu, P., Overman, R. G., Yates, N. L., Alam, S. M., Vandergrift, N., Chen, Y., Graw, F., Freel, S. A., Kappes, J. C., Ochsenbauer, C., et al. (2011). Dynamic antibody specificities and virion concentrations in circulating immune complexes in acute to chronic HIV-1 infection. J Virol 85, 11196-11207.
Montefiori, D. C. (2001). Current Protocols in Immunology (Hoboken, N J, USA: John Wiley & Sons, Inc.).
Moore, J. P., Trkola, A., Korber, B., Boots, L. J., Kessler, J. A., McCutchan, F. E., Mascola, J., Ho, D. D., Robinson, J., and Conley, A. J. (1995). A human monoclonal antibody to a complex epitope in the V3 region of gp120 of human immunodeficiency virus type 1 has broad reactivity within and outside clade B. J Virol 69, 122-130.
Nitayaphan, S., Pitisuttithum, P., Karnasuta, C., Eamsila, C., de Souza, M., Morgan, P., Polonis, V., Benenson, M., VanCott, T., Ratto-Kim, S., et al. (2004). Safety and immunogenicity of an HIV subtype B and E prime-boost vaccine combination in HIV-negative Thai adults. J Infect Dis 190, 702-706.
Rerks-Ngarm, S., Pitisuttithum, P., Nitayaphan, S., Kaewkungwal, J., Chiu, J., Paris, R., Premsri, N., Namwat, C., de Souza, M., Adams, E., et al. (2009). Vaccination with ALVAC and AIDSVAX to prevent HIV-1 infection in Thailand. N Engl J Med 361, 2209-2220.
Rolland, M., Edlefsen, P. T., Larsen, B. B., Tovanabutra, S., Sanders-Buell, E., Hertz, T., Decamp, A. C., Carrico, C., Menis, S., Magaret, C. A., et al. (2012). Increased HIV-1 vaccine efficacy against viruses with genetic signatures in Env V2. Nature 490, 417-420.
Rolland, M., Tovanabutra, S., Decamp, A. C., Frahm, N., Gilbert, P. B., Sanders-Buell, E., Heath, L., Magaret, C.A., Bose, M., Bradfield, A., et al. (2011). Genetic impact of vaccination on breakthrough HIV-1 sequences from the STEP trial. Nat Med 17, 366-371.
Smalls-Mantey, A., Doria-Rose, N., Klein, R., Patamawenu, A., Migueles, S. A., Ko, S.-Y., Hallahan, C. W., Wong, H., Liu, B., You, L., et al. (2012). Antibody-dependent cellular cytotoxicity against primary HIV-infected CD4+ T cells is directly associated with the magnitude of surface IgG binding. J Virol 86, 8672-8680.
Trkola, A., Matthews, J., Gordon, C., Ketas, T., and Moore, J. P. (1999). A cell line-based neutralization assay for primary human immunodeficiency virus type 1 isolates that use either the CCR5 or the CXCR4 coreceptor. J Virol 73, 8966-8974.

The synergistic C and V2 ADCC antibody responses are both dominant responses in that they are readily induced by HIV-1 gp120 envelopes when formulated in Alum, and are expected to be induced by other adjuvants such as AS01B, AS01E or MF59. Thus, polyvalent mixtures of transmitted/founder recombinant gp120 envelopes or their subunits that have been selected, as a group, to mirror overall global H1V-1 viral diversity would be advantageous to use as immunogens. Moreover, deletion of other unrelated dominant regions such as the V3 loop, would be advantageous in order to focus the antibody response on the C1 and the V2 regions. For the V1V2 region, use of smaller Env constructs such as the recombinant V1V2 region in the form of V1V2 tags to focus the antibody response on V2 would be advantageous (Liao H X et al. Immunity 38: 176-186, 2013).

TABLE 4
Dissociation Constants and ADCC Endpoint concentrations of mAbs.

| mAb | Specificity | kₐ (M⁻¹s⁻¹) × 10⁻³ | k_{d} (s⁻¹) × 10⁻³ | K_{d} (nM)* | ADCC EC [μg/ml]** |
|---|---|---|---|---|---|
| A32-IgG | Anti-C1 | 223 | 0.15 | 0.7 | 0.003 |
| CH54-IgG | A32- blockable | 29.1 | 5.35 | 184 | 0.385 |
| CH57-IgG | A32- blockable | 13.6 | 15.6 | 115 | 0.067 |
| CH90-IgG | A32- blockable | 59.0 | 30.0 | 508 | 1.652 |
| CH58-IgG | Anti-V2 | 226 | 0.23 | 1.0 | 9.679 |

*K_{d} was calculated for binding to the AE.A244Δ11 gp120.
**ADCC EC was calculated for AE.CM235-infected target cells by 3 hr Luciferase ADCC.

TABLE 5
Neutralizing activity of mAbs.

| | Inhibition Concentration₅₀ [μg/ml] | | | | | |
|---|---|---|---|---|---|---|
| | | | Clade AE | | | |
| | Clade B | Clade C | | | 427299 | 703357 |
| mAb | MN | TV-1 | 92TH023.6* | CM244 | T/F | T/F |
| A32 | >50 | >50 | >50 | >50 | >50 | >50 |
| CH58 | >50 | >50 | 25.96 | >50 | >50 | >50 |
| CH58 + | >50 | >50 | 0.33 | >50 | >50 | >50 |
| A32 | | | | | | |
| CH59 | >50 | >50 | 5.75 | >50 | >50 | >50 |
| CH59 + | >50 | >50 | <0.023 | >50 | >50 | >50 |
| A32 | | | | | | |
| 4E10** | NT | NT | <0.023 | NT | 1.83 | 9.56 |

*Data are reported as average of 4 replicate experiments. All other clade AE HIV-1 isolates were tested in duplicate experiments.
**The 4E10 mAb was utilized as positive control.

TABLE 6
Synergy by anti-C1 mAb lowers the minimum anti-viral
functional concentrations of anti-V2 mAb CH58.

| Ab Function | Parameter | CH58 alone | CH58 with Anti-C1 |
|---|---|---|---|
| Neutralization | IC50 [μg/ml] | 25.9 | 0.33 |
| ADCC | EC [μg/ml] | 9.7 | 1.10 |
| ADCC | Maximum % killing | 18.8 | 34.5 |

TABLE 7
Combination Index (CI) Values for ADCC activities of vaccine-induced anti-V2
and anti-C1.

| | | | | | Mutually | | Mutually | |
|---|---|---|---|---|---|---|---|---|
| | | | | | Exclusive | | Non-Exclusive | |
| | | | | | (β = 0) | | (β = 1) | |
| | mAb conditions | % Max Killing | EC (μg/ml) | PC75 (μg/ml) | CI EC | CI PC75 | CI EC | CI PC75 |
| CH58 | CH54 (anti-C1) | 37.4 | 0.235 | 1.99 | 1.200 | 4.607 | 1.235 | 4.737 |
| | CH58 (anti-V2) | 18.8 | 9.129 | 31.45 | | | | |
| | V2 in combination | 37.3 | 0.275 | 0.89 | | | | |
| | C1 in combination | 37.3 | 0.275 | 9.12 | | | | |
| | CH57 (anti-C1) | 42.8 | 0.050 | 0.88 | 1.359 | 1.798 | 1.369 | 1.809 |
| | CH58 (anti-V2) | 18.8 | 9.129 | 31.45 | | | | |
| | V2 in combination | 47.0 | 0.068 | 0.19 | | | | |
| | C1 in combination | 47.0 | 0.068 | 1.58 | | | | |
| | CH90 (anti-C1) | 14.5 | 1.642 | 3.85 | 0.815 | 0.419 | 0.901 | 0.439 |
| | CH58 (anti-V2) | 18.8 | 9.129 | 31.45 | | | | |
| | V2 in combination | 34.5 | 1.134 | 1.71 | | | | |
| | C1 in combination | 34.5 | 1.134 | 1.40 | | | | |
| CH59 | CH54 (anti-C1) | 37.4 | 0.235 | 3.69 | 0.374 | 0.120 | 0.408 | 0.124 |
| | CH59 (anti-V2) | 40.1 | 0.174 | 6.82 | | | | |
| | V2 in combination | 61.7 | 0.037 | 0.32 | | | | |
| | C1 in combination | 61.7 | 0.037 | 0.27 | | | | |
| | CH57 (anti-C1) | 42.8 | 0.050 | 0.88 | 1.180 | 0.430 | 1.423 | 0.448 |
| | CH59 (anti-V2) | 40.1 | 0.174 | 6.82 | | | | |
| | V2 in combination | 62.9 | 0.046 | 0.31 | | | | |
| | C1 in combination | 62.9 | 0.046 | 0.34 | | | | |
| | CH90 (anti-C1) | 14.5 | 1.642 | 3.85 | 1.211 | 0.318 | 1.338 | 0.335 |
| | CH59 (anti-V2) | 40.1 | 0.174 | 6.82 | | | | |
| | V2 in combination | 43.4 | 0.191 | 1.70 | | | | |
| | C1 in combination | 43.4 | 0.191 | 0.26 | | | | |
| HG107 | CH54 (anti-C1) | 37.4 | 0.235 | 1.99 | 0.831 | 0.766 | 0.854 | 0.775 |
| | HG107 (anti-V2) | 18.0 | 6.507 | 26.97 | | | | |
| | V2 in combination | 40.1 | 0.188 | 0.33 | | | | |
| | C1 in combination | 40.1 | 0.188 | 1.50 | | | | |
| | CH57 (anti-C1) | 42.8 | 0.050 | 0.88 | 3.355 | 1.708 | 3.441 | 1.725 |
| | HG107 (anti-V2) | 18.0 | 6.507 | 26.97 | | | | |
| | V2 in combination | 45.4 | 0.168 | 0.26 | | | | |
| | C1 in combination | 45.4 | 0.168 | 1.50 | | | | |
| | CH90 (anti-C1) | 14.5 | 1.642 | 3.85 | 0.423 | 0.272 | 0.452 | 0.282 |
| | HG107 (anti-V2) | 18.0 | 6.507 | 26.97 | | | | |
| | V2 in combination | 32.3 | 0.555 | 1.18 | | | | |
| | C1 in combination | 32.3 | 0.555 | 0.88 | | | | |
| HG120 | CH54 (anti-C1) | 37.4 | 0.235 | 1.99 | 0.015 | 0.151 | 0.016 | 0.153 |
| | HG120 (anti-V2) | 32.4 | 1.712 | 16.97 | | | | |
| | V2 in combination | 60.2 | 0.003 | 0.21 | | | | |
| | C1 in combination | 60.2 | 0.003 | 0.28 | | | | |
| | CH57 (anti-C1) | 42.8 | 0.050 | 0.88 | 0.811 | 0.363 | 0.829 | 0.366 |
| | HG120 (anti-V2) | 32.4 | 1.712 | 16.97 | | | | |
| | V2 in combination | 62.5 | 0.040 | 0.15 | | | | |
| | C1 in combination | 62.5 | 0.040 | 0.31 | | | | |
| | CH90 (anti-C1) | 14.5 | 1.642 | 3.85 | 0.226 | 0.116 | 0.239 | 0.119 |
| | HG120 (anti-V2) | 32.4 | 1.712 | 16.97 | | | | |
| | V2 in combination | 46.5 | 0.190 | 0.91 | | | | |
| | C1 in combination | 46.5 | 0.190 | 0.24 | | | | |

All documents and other information sources cited herein are hereby incorporated in their entirety by reference.

1. A composition comprising an isolated anti-V2 (HIV-1 envelope V2) antibody and an isolated anti-CI (HIV-1 envelope C1) antibody.

2. A composition comprising an anti-V2 antibody fragment comprising an antigen binding portion thereof and an anti-CI antibody fragment comprising an antigen binding portion thereof, wherein the composition mediates HIV-1 anti-viral activity.

3. The composition of claim 1, wherein the composition synergistically mediates HIV-1 antiviral activity.

4. The composition of claim 3, wherein the antiviral activity is antibody dependent cellular cytotoxicity.

5. The composition of claim 1, wherein the anti-V2 antibody or fragment thereof comprises a variable heavy chain or a variable light chain from any one of the anti-V2 antibodies described herein.

6. The composition of claim 1, wherein the anti-V2 antibody comprises a CDR from any one of the anti-V2 antibodies described herein.

7. The composition of claim 1, wherein the anti-CI antibody comprises a variable heavy chain or a variable light chain from any one of the anti-CI antibodies described herein.

8. The composition of claim 1, wherein the anti-CI antibody comprises a CDR from any one of the anti-C1 antibodies described herein.

9. The composition of claim 1, wherein the composition comprises an antibody with a variable heavy or a variable light chain from CHS 8 or CH90.

10. The composition of claim 1, wherein the composition comprises antibodies with a variable heavy or a variable light chain from CH58 and CH90.

11. The composition of claim 1, wherein the composition comprises antibodies CH58 and CH90.

12. The composition of claim 1, wherein the antibody is recombinantly produced.

13. An isolated monoclonal anti-V2 antibody or fragment thereof having the binding specificity of any one of antibodies CH58, CH59, HG107 or HG120.

14. An isolated monoclonal anti-CI antibody or fragment thereof having the binding specificity of any one of antibodies CH54, CH57, or CH90.

15. A complementary nucleic acid (cDNA) molecule encoding a variable heavy or light chain from an anti-V2 (HIV envelope V2) antibody or an antigen binding fragment thereof.

16. A complementary nucleic acid (cDNA) molecule encoding a variable heavy or light chain from an anti-CI antibody or an antigen binding fragment thereof.

17. A vector comprising the cDNA of claim 15.

18. A host cell comprising the vector of claim 17.

19. A polypeptide comprising the amino acid sequence of an anti-V2 antibody or an antigen binding fragment thereof.

20. A polypeptide comprising the amino acid sequence of an anti-C1 antibody or an antigen binding fragment thereof.

21. A polypeptide comprising the amino acid sequence or a fragment thereof of any one of the antibodies described herein.

22. An HIV-1 prophylactic method comprising administering to a subject a composition of claim 1 in an amount sufficient to reduce the risk or prevent an HIV infection.
